(19) United States
(12) Patent Application Publication
(10) Pub. No.: US 2004/0214348 A1
(43) Pub. Date: Oct. 28, 2004

(54) Methods for analysis of spectral data and their applications: osteoarthritis

(76) Inventors: Jeremy Kirk Nicholson, Croydon (GB); Elaine Holmes, London (GB); John Christopher Lindon, Westerham (GB); Joanne Tracey Brindle, Watchfield (GB); David John Grainger, Cambridge (GB)

Correspondence Address: Robert K Cerpa, Morrison & Foerster, 755 Page Mill Road, Palo Alto, CA, 94304-1018, US

(21) Appl. No.: 10/475,733

(22) PCT Filed: Apr. 23, 2002
(86) PCT No.: PCT/GB02/01928

(51) Int. Cl. G01N 24/00; G01N 33/543

(52) U.S. Cl. 436/518; 436/173

(57) ABSTRACT

This invention pertains to chemometric methods for the analysis of chemical, biochemical, and biological data, for example, spectral data, for example, nuclear magnetic resonance (NMR) spectra, and their applications, including, e.g., classification, diagnosis, prognosis, etc., especially in the context of osteoarthritis

### RELATED APPLICATIONS

This application is related to (and where permitted by law, claims priority to):

(a) United Kingdom patent application GB 01 09930.8 filed 23 Apr. 2001;

(b) United Kingdom patent application GB 0117428.3 filed 17 Jul. 2001;

(c) U.S. Provisional patent application U.S.SNo. 60/307,015 filed 20 Jul. 2001;

the contents of each of which are incorporated herein by reference in their entirety.

This application is one of five applications filed on even date naming the same applicant:

(1) attorney reference number WJW/LP5995600 (PCT/GB02/______);

(2) attorney reference number WJW/LP5995618 (PCT/GB02/______);

(3) attorney reference number WJW/LP5995626 (PCT/GB02/______);

(4) attorney reference number WJW/LP5995634 (PCT/GB02/______);

(5) attorney reference number WJW/LP5995642 (PCT/GB02/______);

the contents of each of which are incorporated herein by reference in their entirety.

### TECHNICAL FIELD

This invention pertains generally to the field of metabonomics, and, more particularly, to chemometric methods for the analysis of chemical, biochemical, and biological data, for example, spectral data, for example, nuclear magnetic resonance (NMR) spectra, and their applications, including, e.g., classification, diagnosis, prognosis, etc., especially in the context of osteoarthritis.

### BACKGROUND

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Biosystems

Biosystems can conveniently be viewed at several levels of bio-molecular organisation based on biochemistry, i.e., genetic and gene expression (genomic and transcriptomic), protein and signalling (proteomic) and metabolic control and regulation (metabonomic). There are also important cellular ionic regulation variations that relate to genetic, proteomic and metabolic activities, and systematic studies on these even at the cellular and sub-cellular level should also be investigated to complete the full description of the bio-molecular organisation of a bio-system.

Significant progress has been made in developing methods to determine and quantify the biochemical processes occurring in living systems. Such methods are valuable in the diagnosis, prognosis and treatment of disease, the development of drugs, for improving therapeutic regimes for current drugs, and the like.

Many diseases of the human or animal body (such as cancers, degenerative diseases, autoimmune diseases and the like) have an underlying basis in alterations in the expression of certain genes. The expressed gene products, proteins, mediate effects such as abnormal cell growth, cell death or inflammation. Some of these effects are caused directly by protein-protein interactions; other are caused by proteins acting on small molecules (e.g. “second messengers”) which trigger effects including further gene expression.

Likewise, disease states caused by external agents such as viruses and bacteria provoke a multitude of complex responses in infected host.

In a similar manner, the treatment of disease through the administration of drugs can result in a wide range of desired effects and unwanted side effects in a patient.

In recent years, it has been appreciated that the reaction of human and animal subjects to disease and treatments for them can vary according to the genomic makeup of an individual. This has led to the development of the field of “pharmacogenomics.” A fuller understanding of how an individual's own genome reacts to a particular disease and/or drug treatment will allow the development of new therapies, as well as the refinement of existing ones.

At the genetic level, methods for examining gene expression in response to these types of events are often referred to as “genomic methods,” and are concerned with the detection and quantification of the expression of an organism's genes, collectively referred to as its “genome,” usually by detecting and/or quantifying genetic molecules, such as DNA and RNA. Genomic studies often exploit proprietary “gene chips,” which are small disposable devices encoded with an array of genes that respond to extracted mRNAs produced by cells (see, for example, Klenk et al., 1997). Many genes can be placed on a chip array and patterns of gene expression, or changes therein, can be monitored rapidly, although at some considerable cost.

However, the biological consequences of gene expression, or altered gene expression following perturbation, are extremely complex. This has led to the development of “proteomic methods” which are concerned with the semi-quantitative measurement of the production of cellular proteins of an organism, collectively referred to as its “proteome” (see, for example, Geisow, 1998). Proteomic measurements utilise a variety of technologies, but all involve a protein separation method, e.g., 2D gel-electrophoresis, allied to a chemical characterisation method, usually, some form of mass spectrometry.

At present, genomic methods have a high associated operational cost and proteomic methods require investment in expensive capital cost equipment and are labour intensive, but both have the potential to be powerful tools for studying biological response. The choice of method is still uncertain since careful studies have sometimes shown a low correlation between the pattern of gene expression and the pattern of protein expression, probably due to sampling for the two technologies at inappropriate time points. See, e.g., Gygi et al., 1999. Even in combination, genomic and proteomic methods still do not provide the range of information needed for understanding integrated cellular function in a living system, since they do not take account of the dynamic metabolic status of the whole organism.

For example, genomic and proteomic studies may implicate a particular gene or protein in a disease or a xenobiotic response because the level of expression is altered, but the change in gene or protein level may be transitory or may be counteracted downstream and as a result there may be no effect at the cellular and/or biochemical level. Conversely, sampling tissue for genomic and proteomic studies at inappropriate time points may result in a relevant gene or protein being overlooked.

Gene-based prognosis has yet to become a clinical reality for any major prevalent disease, almost all of which have multi-gene modes of inheritance and significant environmental impact making it difficult to identify the gene panels responsible for susceptibility.

While genomic and proteomic methods may be useful aids, for example, in drug development, they do suffer from substantial limitations. For example, while genomic and proteomic methods may ultimately give profound insights into toxicological mechanisms and provide new surrogate biomarkers of disease, at present it is very difficult to relate genomic and proteomic findings to classical cellular or biochemical indices or endpoints. One simple reason for this is that with current technology and approach, the correlation of the time-response to drug exposure is difficult. Further difficulties arise with in vitro cell-based studies. These difficulties are particularly important for the many known cases where the metabolism of the compound is a prerequisite for a toxic effect and especially true where the target organ is not the site of primary metabolism. This is particularly true for pro-drugs, where some aspect of in situ chemical (e.g., enzymatic) modification is required for activity.

Metabonomics

A new “metabonomic” approach has been developed which is aimed at augmenting and complementing the information provided by genomics and proteomics. “Metabonomics” is conventionally defined as “the quantitative measurement of the multiparametric metabolic response of living systems to pathophysiological stimuli or genetic modification” (see, for example, Nicholson et al., 1999). This concept has arisen primarily from the application of ¹H NMR spectroscopy to study the metabolic composition of biofluids, cells, and tissues and from studies utilising pattern recognition (PR), expert systems and other chemoinformatic tools to interpret and classify complex NMR-generated metabolic data sets. Metabonomic methods have the potential, ultimately, to determine the entire dynamic metabolic make-up of an organism.

As outlined above, each level of bio-molecular organisation requires a series of analytical bio-technologies appropriate to the recovery of the individual types of bio-molecular data. Genomic, proteomic and metabonomic technologies by definition generate massive data sets which require appropriate multi-variate statistical tools (chemometrics, bio-informatics) for data mining and to extract useful biological information. These data exploration tools also allow the inter-relationships between multivariate data sets from the different technologies to be investigated, they facilitate dimension reduction and extraction of latent properties and allow multidimensional visualization.

This leads to the concept of “bionomics”, the quantitative measurement and understanding of the integrated function (and dysfunction)of biological systems at all major levels of bio-molecular organisation. In the study of altered gene expression, (known as transcriptomics), the variables are mRNA responses measured using gene chips, in proteomics, protein synthesis and asociated post-translational modifications are typically measured using (mainly) gel-electrophoresis coupled to mass spectrometry. In both cases, thousands of variables can be measured and related to biological end-points using statistical methods. In metabolic (metabonomic) studies, only NMR (especially ¹H) and mass spectrometry has been used to provide this level of data density on bio-materials although these data can be supplemented by conventional biochemical assays.

For in vivo mammalian studies, the ability to perform metabonomic studies on biofluids such as plasma, CSF and urine is very important because it gives integrated systems-based information on the whole organism. Furthermore, in clinical settings, for the full utilization of functional genomic knowledge in patient screening, diagnostics and prognostics, it is much more practical and ethically-acceptable to analyze biofluid samples than to perform human tissue biopsies and measure gene responses.

A pathological condition or a xenobiotic may act at the pharmacological level only and hence may not affect gene regulation or expression directly. Alternatively significant disease or toxicological effects may be completely unrelated to gene switching. For example, exposure to ethanol in vivo may cause many changes in gene expression but none of these events explains drunkenness. In cases such as these, genomic and proteomic methods are likely to be ineffective. However, all disease or drug-induced pathophysiological perturbations result in disturbances in the ratios and concentrations, binding or fluxes of endogenous biochemicals, either by direct chemical reaction or by binding to key enzymes or nucleic acids that control metabolism. If these disturbances are of sufficient magnitude, effects will result which will affect the efficient functioning of the whole organism. In body fluids, metabolites are in dynamic equilibrium with those inside cells and tissues and, consequently, abnormal cellular processes in tissues of the whole organism following a toxic insult or as a consequence of disease will be reflected in altered biofluid compositions.

Fluids secreted, excreted, or otherwise derived from an organism (“biofluids”) provide a unique window into its biochemical status since the composition of a given biofluid is a consequence of the function of the cells that are intimately concerned with the fluid's manufacture and secretion. For example, the composition of a particular fluid (e.g., urine, blood plasma, milk, etc.) can carry biochemical information on details of organ function (or dysfunction), for example, as a result of xenobiotics, disease, and/or genetic modification. Similarly, the composition and condition of an organism's tissues are also indicators of the organism's biochemical status.

In general, a xenobiotic is a substance (e.g., compound, composition) which is administered to an organism, or to which the organism is exposed. In general, xenobiotics are chemical, biochemical or biological species (e.g., compounds) which are not normally present in that organism, or are normally present in that organism, but not at the level obtained following administration/exposure. Examples of xenobiotics include drugs, formulated medicines and their components (e.g., vaccines, immunological stimulants, inert carrier vehicles), infectious agents, pesticides, herbicides, substances present in foods (e.g. plant compounds administered to animals), and substances present in the environment.

In general, a disease state pertains to a deviation from the normal healthy state of the organism. Examples of disease states include, but are not limited to, bacterial, viral, and parasitic infections; cancer in all its forms; degenerative diseases (e.g., arthritis, multiple sclerosis); trauma (e.g., as a result of injury); organ failure (including diabetes); cardiovascular disease (e.g., atherosclerosis, thrombosis); and, inherited diseases caused by genetic composition (e.g., sickle-cell anaemia).

In general, a genetic modification pertains to alteration of the genetic composition of an organism. Examples of genetic modifications include, but are not limited to: the incorporation of a gene or genes into an organism from another species; increasing the number of copies of an existing gene or genes in an organism; removal of a gene or genes from an organism; and, rendering a gene or genes in an organism non-functional.

Biofluids often exhibit very subtle changes in metabolite profile in response to external stimuli. This is because the body's cellular systems attempt to maintain homeostasis (constancy of internal environment), for example, in the face of cytotoxic challenge. One means of achieving this is to modulate the composition of biofluids. Hence, even when cellular homeostasis is maintained, subtle responses to disease or toxicity are expressed in altered biofluid composition. However, dietary, diurnal and hormonal variations may also influence biofluid compositions, and it is clearly important to differentiate these effects if correct biochemical inferences are to be drawn from their analysis.

Metabonomics offers a number of distinct advantages (over genomics and proteomics) in a clinical setting: firstly, it can often be performed on standard preparations (e.g., of serum, plasma, urine, etc.), circumventing the need for specialist preparations of cellular RNA and protein required for genomics and proteomics, respectively. Secondly, many of the risk factors already identified (e.g., levels of various lipids in blood) are small molecule metabolites which will contribute to the metabonomic dataset.

Application of NMR to Metabonomics

One of the most successful approaches to biofluid analysis has been the use of NMR spectroscopy (see, for example, Nicholson et al., 1989); similarly, intact tissues have been successfully analysed using magic-angle-spinning ¹H NMR spectroscopy (see, for example, Moka et al., 1998; Tomlins et al., 1998).

The NMR spectrum of a biofluid provides a metabolic fingerprint or profile of the organism from which the biofluid was obtained, and this metabolic fingerprint or profile is characteristically changed by a disease, toxic process, or genetic modification. For example, NMR spectra may be collected for various states of an organism (e.g., pre-dose and various times post-dose, for one or more xenobiotics, separately or in combination; healthy (control) and diseased animal; unmodified (control) and genetically modified animal).

For example, in the evaluation of undesired toxic side-effects of drugs, each compound or class of compound produces characteristic changes in the concentrations and patterns of endogenous metabolites in biofluids that provide information on the sites and basic mechanisms of the toxic process. ¹H NMR analysis of biofluids has successfully uncovered novel metabolic markers of organ-specific toxicity in the laboratory rat, and it is in this “exploratory” role that NMR as an analytical biochemistry technique excels. However, the biomarker information in NMR spectra of biofluids is very subtle, as hundreds of compounds representing many pathways can often be measured simultaneously, and it is this overall metabonomic response to toxic insult that so well characterises the lesion.

Another important advantage of NMR-based metabonomics over genomics or proteomics is the intrinsic analytical accuracy of NMR spectroscopy. Reanalysis of the same sample by 1H NMR spectroscopy results in a typical coefficient of variation for the measurement of peak intensities in a spectrum of less than 5% across the whole range of peaks. Thus if the appropriate experiments are undertaken, on average the value of each peak intensity will lie in the range 0.95 to 1.05 of the true value. In addition, it is possible using NMR spectroscopy to measure absolute amounts or concentrations of a number of analytes whereas using gene chip technology only fold changes can be determined. The best available accuracy achieved using gene chips is a two fold change, i.e., the value for each parameter lies in the range 0.50 to 2.00 fold of the “true” value) and proteomic technology is even less intrinsically accurate. A similar limitation also applies to proteomic studies.

Although, undoubtedly, technology is improving at a rapid rate the gap between the intrinsic accuracies of NMR spectroscopy and gene chip technology is so wide that it will require a revolutionary rather than evolutionary improvement in gene expression quantification methodology before it can rival the accuracy of NMR spectroscopy.

The intrinsic accuracy of NMR provides a distinct advantage when applying pattern recognition techniques. The multivariate nature of the NMR data means that classification of samples is possible using a combination of descriptors even when one descriptor is not sufficient, because of the inherently low analytical variation in the data.

All biological fluids and tissues have their own characteristic physico-chemical properties, and these affect the types of NMR experiment that may be usefully employed. One major advantage of using NMR spectroscopy to study complex biomixtures is that measurements can often be made with minimal sample preparation (usually with only the addition of 5-10% D₂O) and a detailed analytical profile can be obtained on the whole biological sample. Sample volumes are small, typically 0.3 to 0.5 mL for standard probes, and as low as 3 μL for microprobes. Acquisition of simple NMR spectra is rapid and efficient using flow-injection technology. It is usually necessary to suppress the water NMR resonance.

Many biofluids are not chemically stable and for this reason care should be taken in their collection and storage. For example, cell lysis in erythrocytes can easily occur. If a substantial amount of D₂O has been added, then it is possible that certain ¹H NMR resonances will be lost by H/D exchange. Freeze-drying of biofluid samples also causes the loss of volatile components such as acetone. Biofluids are also very prone to microbiological contamination, especially fluids, such as urine, which are difficult to collect under sterile conditions. Many biofluids contain significant amounts of active enzymes, either normally or due to a disease state or organ damage, and these enzymes may alter the composition of the biofluid following sampling. Samples should be stored deep frozen to minimise the effects of such contamination. Sodium azide is usually added to urine at the collection point to act as an antimicrobial agent. Metal ions and or chelating agents (e.g., EDTA) may be added to bind to endogenous metal ions (e.g., Ca²⁺, Mg²⁺ and Zn²⁺) and chelating agents (e.g., free amino acids, especially glutamate, cysteine, histidine and aspartate; citrate) to intentionally alter and/or enhance the NMR spectrum.

In all cases the analytical problem usually involves the detection of “trace” amounts of analytes in a very complex matrix of potential interferences. It is, therefore, critical to choose a suitable analytical technique for the particular class of analyte of interest in the particular biomatrix which could be, for example, a biofluid or a tissue. High resolution NMR spectroscopy (in particular ¹H NMR) appears to be particularly appropriate. The main advantages of using ¹H NMR spectroscopy in this area are the speed of the method (with spectra being obtained in 5 to 10 minutes), the requirement for minimal sample preparation, and the fact that it provides a non-selective detector for all metabolites in the biofluid regardless of their structural type, provided only that they are present above the detection limit of the NMR experiment and that they contain non-exchangeable hydrogen atoms. The speed advantage is of crucial importance in this area of work as the clinical condition of a patient may require rapid diagnosis, and can change very rapidly and so correspondingly rapid changes must be made to the therapy provided.

NMR studies of body fluids should ideally be performed at the highest magnetic field available to obtain maximal dispersion and sensitivity and most ¹H NMR studies have been performed at 400 MHz or greater. With every new increase in available spectrometer frequency the number of resonances that can be resolved in a biofluid increases and although this has the effect of solving some assignment problems, it also poses new ones. Furthermore, there are still important problems of spectral interpretation that arise due to compartmentation and binding of small molecules in the organised macromolecular domains that exist in some biofluids such as blood plasma and bile. All this complexity need not reduce the diagnostic capabilities and potential of the technique, but demonstrates the problems of biological variation and the influence of variation on diagnostic certainty.

The information content of biofluid spectra is very high and the complete assignment of the ¹H NMR spectrum of most biofluids is usually not possible (even using 900 MHz NMR spectroscopy). However, the assignment problems vary considerably between biofluid types. Some fluids have near constant composition and concentrations and in these the majority of the NMR signals have been assigned. In contrast, urine composition can be very variable and there is enormous variation in the concentration range of NMR-detectable metabolites; consequently, complete analysis is much more difficult. Those metabolites present close to the limits of detection for 1-dimensional (1D) NMR spectroscopy (typically ca. 100 nM at 800 MHz) pose severe NMR spectral assignment problems. (In absolute terms, the detection limit may be ca. 4 nmol, e.g., 1 μg of a 250 g/mol compound in a 0.5 mL sample volume.) Even at the present level of technology in NMR, it is not yet possible to detect many important biochemical substances (e.g. hormones, some proteins, nucleic acids) in body fluids because of problems with sensitivity, line widths, dispersion and dynamic range and this area of research will continue to be technology-limited. In addition, the collection of NMR spectra of biofluids may be complicated by the relative water intensity, sample viscosity, protein content, lipid content, and low molecular weight peak overlap.

Usually in order to assign ¹H NMR spectra, comparison is made with spectra of authentic materials and/or by standard addition of an authentic reference standard to the sample. Additional confirmation of assignments is usually sought from the application of other NMR methods, including, for example, 2-dimensional (2D) NMR methods, particularly COSY (correlation spectroscopy), TOCSY (total correlation spectroscopy), inverse-detected heteronuclear correlation methods such as HMBC (heteronuclear multiple bond correlation), HSQC (heteronuclear single quantum coherence), and HMQC (heteronuclear multiple quantum coherence), 2D J-resolved (JRES) methods, spin-echo methods, relaxation editing, diffusion editing (e.g., both 1D NMR and 2D NMR such as diffusion-edited TOCSY), and multiple quantum filtering. Detailed ¹H NMR spectroscopic data for a wide range of metabolites and biomolecules found in biofluids have been published (see, for example, Lindon et al., 1999) and supplementary information is available in several literature compilations of data (see, for example, Fan, 1996; Sze et al., 1994).

For example, the successful application of ¹H NMR spectroscopy of biofluids to study a variety of metabolic diseases and toxic processes has now been well established and many novel metabolic markers of organ-specific toxicity have been discovered (see, for example, Nicholson et al., 1989; Lindon et al., 1999). For example, NMR spectra of urine is identifiably altered in situations where damage has occurred to the kidney or liver. It has been shown that specific and identifiable changes can be observed which distinguish the organ that is the site of a toxic lesion. Also it is possible to focus in on particular parts of an organ such as the cortex of the kidney and even in favourable cases to very localised parts of the cortex.

It is also possible to deduce the biochemical mechanism of the xenobiotic toxicity, based on a biochemical interpretation of the changes in the urine. A wide range of toxins has now been investigated including mostly kidney toxins and liver toxins, but also testicular toxins, mitochondrial toxins and muscle toxins.

Pattern Recognition

However, a limiting factor in understanding the biochemical information from both 1D and 2D-NMR spectra of tissues and biofluids is their complexity. The most efficient way to investigate these complex multiparametric data is employ the 1D and 2D NMR metabonomic approach in combination with computer-based “pattern recognition” (PR) methods and expert systems. These statistical tools are similar to those currently being explored by workers in the fields of genomics and proteomics.

Pattern recognition (PR) methods can be used to reduce the complexity of data sets, to generate scientific hypotheses and to test hypotheses. In general, the use of pattern recognition algorithms allows the identification, and, with some methods, the interpretation of some non-random behaviour in a complex system which can be obscured by noise or random variations in the parameters defining the system. Also, the number of parameters used can be very large such that visualisation of the regularities, which for the human brain is best in no more than three dimensions, can be difficult. Usually the number of measured descriptors is much greater than three and so simple scatter plots cannot be used to visualise any similarity between samples. Pattern recognition methods have been used widely to characterise many different types of problem ranging for example over linguistics, fingerprinting, chemistry and psychology. In the context of the methods described herein, pattern recognition is the use of multivariate statistics, both parametric and non-parametric, to analyse spectroscopic data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. The other approach is termed “supervised” whereby a training set of samples with known class or outcome is used to produce a mathematical model and this is then evaluated with independent validation data sets.

Unsupervised PR methods are used to analyse data without reference to any other independent knowledge, for example, without regard to the identity or nature of a xenobiotic or its mode of action. Examples of unsupervised pattern recognition methods include principal component analysis (PCA), hierarchical duster analysis (HCA), and non-linear mapping (NLM).

One of the most useful and easily applied unsupervised PR techniques is principal components analysis (PCA) (see, for example, Kowalski et al, 1986). Principal components (PCs) are new variables created from linear combinations of the starting variables with appropriate weighting coefficients. The properties of these PCs are such that: (i) each PC is orthogonal to (uncorrelated with) all other PCs, and (ii) the first PC contains the largest part of the variance of the data set (information content) with subsequent PCs containing correspondingly smaller amounts of variance.

PCA, a dimension reduction technique, takes m objects or samples, each described by values in K dimensions (descriptor vectors), and extracts a set of eigenvectors, which are linear combinations of the descriptor vectors. The eigenvectors and eigenvalues are obtained by diagonalisation of the covariance matrix of the data. The eigenvectors can be thought of as a new set of orthogonal plotting axes, called principal components (PCs). The extraction of the systematic variations in the data is accomplished by projection and modelling of variance and covariance structure of the data matrix. The primary axis is a single eigenvector describing the largest variation in the data, and is termed principal component one (PC1). Subsequent PCs, ranked by decreasing eigenvalue, describe successively less variability. The variation in the data that has not been described by the PCs is called residual variance and signifies how well the model fits the data. The projections of the descriptor vectors onto the PCs are defined as scores, which reveal the relationships between the samples or objects. In a graphical representation (a “scores plot” or eigenvector projection), objects or samples having similar descriptor vectors will group together in clusters. Another graphical representation is called a loadings plot, and this connects the PCs to the individual descriptor vectors, and displays both the importance of each descriptor vector to the interpretation of a PC and the relationship among descriptor vectors in that PC. In fact, a loading value is simply the cosine of the angle which the original descriptor vector makes with the PC. Descriptor vectors which fall close to the origin in this plot carry little information in the PC, while descriptor vectors distant from the origin (high loading) are important in interpretation.

Thus a plot of the first two or three PC scores gives the “best” representation, in terms of information content, of the data set in two or three dimensions, respectively. A plot of the first two principal component scores, PC1 and PC2 provides the maximum information content of the data in two dimensions. Such PC maps can be used to visualise inherent clustering behaviour, for example, for drugs and toxins based on similarity of their metabonomic responses and hence mechanism of action. Of course, the clustering information might be in lower PCs and these have also to be examined.

Hierarchical Cluster Analysis, another unsupervised pattern recognition method, permits the grouping of data points which are similar by virtue of being “near” to one another in some multidimensional space. Individual data points may be, for example, the signal intensities for particular assigned peaks in an NMR spectrum. A “similarity matrix,” S, is constructed with elements sᵢⱼ=1−rᵢⱼ/rᵢⱼ^{max}, where rᵢⱼ is the interpoint distance between points i and j (e.g., Euclidean interpoint distance), and rᵢⱼ^{max} is the largest interpoint distance for all points. The most distant pair of points will have sᵢⱼ equal to 0, since rᵢⱼ then equals rᵢⱼ^{max}. Conversely, the closest pair of points will have the largest sᵢⱼ. For two identical points, sᵢⱼ is 1.

The similarity matrix is scanned for the closest pair of points. The pair of points are reported with their separation distance, and then the two points are deleted and replaced with a single combined point. The process is then repeated iteratively until only one point remains. A number of different methods may be used to determine how two clusters will be joined, including the nearest neighbour method (also known as the single link method), the furthest neighbour method, and the centroid method (including centroid link, incremental link, median link, group average link, and flexible link variations).

The reported connectivities are then plotted as a dendrogram (a tree-like chart which allows visualisation of clustering), showing sample-sample connectivities versus increasing separation distance (or equivalently, versus decreasing similarity). The dendrogram has the property in which the branch lengths are proportional to the distances between the various clusters and hence the length of the branches linking one sample to the next is a measure of their similarity. In this way, similar data points may be identified algorithmically.

Non-linear mapping (NLM) is a simple concept which involves calculation of the distances between all of the points in the original K dimensions. This is followed by construction of a map of points in 2 or 3 dimensions where the sample points are placed in random positions or at values determined by a prior principal components analysis. The least squares criterion is used to move the sample points in the lower dimension map to fit the inter-point distances in the lower dimension space to those in the K dimensional space. Non-linear mapping is therefore an approximation to the true inter-point distances, but points close in K-dimensional space should also be close in 2 or 3 dimensional space (see, for example, Brown et al., 1996; Farrant et al., 1992).

In this simple metabonomic approach, a sample from an animal treated with a compound of unknown toxicity is compared with a database of NMR-generated metabolic data from control and toxin-treated animals. By observing its position on the PR map relative to samples of known effect, the unknown toxin can often be classified. The same approach can be used for human samples for classification according to disease. However, such data are often more complex, with time-related biochemical changes detected by NMR. Also, it is more rigorous to compare effects of xenobiotics in the original K-dimensional NMR metabonomic space.

Alternatively, and in order to develop automatic classification methods, it has proved efficient to use a “supervised” approach to NMR data analysis. Here, a “training set” of NMR metabonomic data is used to construct a statistical model that predicts correctly the “class” of each sample. This training set is then tested with independent data (referred to as a test or validation set) to determine the robustness of the computer-based model. These models are sometimes termed “expert systems,” but may be based on a range of different mathematical procedures. Supervised methods can use a data set with reduced dimensionality (for example, the first few principal components), but typically use unreduced data, with all dimensionality. In all cases the methods allow the quantitative description of the multivariate boundaries that characterise and separate each class, for example, each class of xenobiotic in terms of its metabolic effects. It is also possible to obtain confidence limits on any predictions, for example, a level of probability to be placed on the goodness of fit (see, for example, Kowalski et al., 1986). The robustness of the predictive models can also be checked using cross-validation, by leaving out selected samples from the analysis.

Expert systems may operate to generate a variety of useful outputs, for example, (i) classification of the sample as “normal” or “abnormal” (this is a useful tool in the control of spectrometer automation, e.g., using sequential flow injection NMR spectroscopy); (ii) classification of the target organ for toxicity and site of action within the tissue where in certain cases, mechanism of toxic action may also be classified; and, (iii) identification of the biomarkers of a pathological disease condition or toxic effect for the particular compound under study. For example, a sample can be classified as belonging to a single class of toxicity, to multiple classes of toxicity (more than one target organ), or to no class. The latter case would indicate deviation from normality (control) based on the training set model but having a dissimilar metabolic effect to any toxicity class modelled in the training set (unknown toxicity type). Under (ii), a system could also be generated to support decisions in clinical medicine (e.g., for efficacy of drugs) rather than toxicity.

Examples of supervised pattern recognition methods include the following:

soft independent modelling of class analysis (SIMCA) (see, for example, Wold, 1976);

partial least squares analysis (PLS) (see, for example, Wold, 1966; Joreskog, 1982; Frank, 1984; Bro, R., 1997);

linear descriminant analysis (LDA) (see, for example, Nillson, 1965);

K-nearest neighbour analysis (KNN) (see, for example, Brown et al., 1996);

artificial neural networks (ANN) (see, for example, Wasserman, 1989; Anker et al., 1992; Hare, 1994);

probabilistic neural networks (PNNs) (see, for example, Parzen, 1962; Bishop, 1995; Speckt, 1990; Broomhead et al., 1988; Patterson, 1996);

rule induction (RI) (see, for example, Quinlan, 1986); and,

Bayesian methods (see, for example, Bretthorst, 1990a, 1990b, 1988).

As the size of metabonomic databases increases together with improvements in rapid throughput of NMR samples (>300 samples per day per spectrometer is now possible with the first generation of flow injection systems), more subtle expert systems may be necessary, for example, using techniques such as “fuzzy logic” which permit greater flexibility in decision boundaries.

Application to Metabonomics

Pattern recognition methods have been applied to the analysis of metabonomic data. See, for example, Lindon et al., 2001. A number of spectroscopic techniques have been used to generate the data, including NMR spectroscopy and mass spectrometry. Pattern recognition analysis of such data sets has been succesful in some cases. The successful studies include, for example, complex NMR data from biofluids, (see, for example, Anthony et al., 1994; Anthony et al., 1995; Beckwith-Hall et al., 1998; Gartland et al., 1990a; Gartland et al., 1990b; Gartland et al., 1991; Holmes et al., 1998a; Holmes et al., 1998b; Holmes et al., 1992; Holmes et al., 1994; Spraul et al., 1994; Tranter et al., 1999) conventional NMR spectra from tissue samples (Somorjai et al., 1995), magic-angle-spinning (MAS) NMR spectra of tissues (Garrod et al., 2001), in vivo NMR spectra (Morvan et al., 1990; Howells et al., 1993; Stoyanova et al., 1995; Kuesel et al., 1996; Confort-Gouny et al., 1992; Weber et al., 1998), wines (Martin et al., 1998, 1999) and plant tissues (Kopka et al., 2000).

Although the utility of the metabonomic approach is well established, its full potential has not yet been exploited. The metabolic variation is often subtle, and powerful analysis methods are required for detection of particular analytes, especially when the data (e.g., NMR spectra) are so complex. For example, all that has been previously proposed is still not generally sufficient to achieve clinically useful diagnosis of disease. New methods to extract useful metabolic information from biofluids are needed.

The inventors have developed novel methods (which employ multivariate statistical analysis and pattern recognition (PR) techniques, and optionally data filtering techniques) of analysing data (e.g., NMR spectra) from a test population which yield accurate mathematical models which may subsequently be used to classify a test sample or subject, and/or in diagnosis.

Unlike methods previously described, the methods described herein have the power to provide clinically useful and accurate diagnostic and prognostic information in a medical setting.

The methods described herein represent a significant advance over chemometric methodologies described previously. Although chemometrics has been able to provide some classification of types previously, the studies have required that the classification be done under a series of restrictions which limit the ability to apply the method to analysis of complex datasets as would be required to apply the method for the practical diagnosis/prognosis of diseases that could be useful clinically.

For example, several studies have reported on the classification of animals on the basis of an NMR spectrum of urine or plasma. Although these studies clearly demonstrate the potential of the technique, they are limited because the animals which compose each class are genetically homogenous (in-bred populations). As a result, these methods have been demonstrated to be able to detect patterns but only against “low noise” backgrounds. Application of metabonomics to “real” populations (e.g., in human clinical practice) requires the ability to detect patterns against the substantial noise due to the genetic variation of out-bred populations and also due to dietary and hormonal differences.

Similarly, many of the studies described to date have examined relatively major differences between groups, for example, the ability to differentiate renally acting toxins from liver acting toxins. The two groups under study differed in a broad spectrum of metabolites making the pattern relatively easy to detect. In conjugation with the restriction of using in-bred populations of animals, most studies published to date have only demonstrated metabonomics to be practicable under conditions of high “signal to noise” ratio, conditions which are very different from the human clinical environment.

Some studies have begun to attempt classifications of out-bred human populations where the data variation is high. However, to date, all these studies have simplified the system substantially to focus in on specific molecules: for example, some studies have looked specifically at the resonances associated with lipoproteins. Since lipoproteins are major constituents of plasma, the variance they contribute readily exceeds the background variance due to genetic and environmental differences between individuals. Unfortunately, such an approach is insufficiently powerful to identify weak patterns against the background biochemical noise, and could not be used, for example, to determine the extent of coronary heart disease or to distinguish identical from non-identical twins. Identification of such low “signal to noise” ratio patterns requires the application of the methods of this invention, which represent a significant advance over what has been previously reported.

### SUMMARY OF THE INVENTION

One aspect of the present invention pertains to a method of classifying a sample, as described herein.

One aspect of the present invention pertains to a method of classifying a subject as described herein.

One aspect of the present invention pertains to a method of diagnosing a subject as described herein.

One aspect of the present invention pertains to a method of identifying a diagnostic species, or a combination of a plurality of diagnostic species, for a predetermined condition, as described herein.

One aspect of the present invention pertains to a diagnostic species identified by a method as described herein.

One aspect of the present invention pertains to a diagnostic species identified by a method as described herein, for use in a method of classification.

One aspect of the present invention pertains to a method of classification which employs or relies upon one or more diagnostic species identified by a method as described herein

One aspect of the present invention pertains to use of one or more diagnostic species identified by a method of classification as described herein.

One aspect of the present invention pertains to an assay for use in a method -of classification, which assay relies upon one or more diagnostic species identified by a method as described herein.

One aspect of the present invention pertains to use of an assay in a method of classification, which assay relies upon one or more diagnostic species identified by a method as described herein.

One aspect of the present invention pertains to a method of therapeutic monitoring of a subject undergoing therapy which employs a method of classification as described herein.

One aspect of the present invention pertains to a method of evaluating drug therapy and/or drug efficacy which employs a method of classification, as described herein.

One aspect of the present invention pertains to a computer system or device, such as a computer or linked computers, operatively configured to implement a method as described herein; and related computer code computer programs, data carriers carrying such code and programs, and the like.

These and other aspects of the present invention are described herein.

As will be appreciated by one of skill in the art, features and preferred embodiments of one aspect of the present invention will also pertain to other aspects of the present invention.

### BRIEF DESCRIPTION OF THE DRAWINGS

FIG. 1A-OA is a scores scatter plot for PC2 and PC1 (t2 vs. t1) for the principal components analysis (PCA) model derived from 1-D ¹H NMR spectra from serum samples from control subjects (triangles, ▴) and patients with osteoarthritis. (circles, ●).

FIG. 1B-OA is the corresponding loadings scatter plot (p2 vs. p1) for the PCA shown in FIG. 1A-OA.

FIG. 1C-OA is a scores scatter plot for PC3 and PC2 (t3 vs. t2) for the PCA model derived from 1-D ¹H NMR spectra from serum samples from control subjects (triangles, ▴) and patients with osteoarthritis (circles, ●). Prior to PCA, the data were filtered (in this case, using orthogonal signal correction, OSC).

FIG. 1D-OA is the corresponding loadings scatter plot (p3 vs. p2) for the PCA shown in FIG. 1C-OA.

FIG. 1E-OA is a scores scatter plot for PC2 and PC1 (t2 vs. t1) for the PLS-DA model derived from 1-D ¹H NMR spectra from serum samples from control subjects (triangles, ▴) and patients with osteoarthritis (circles, ●). Prior to PLS-DA, the data were filtered (in this case, using orthogonal signal correction, OSC).

FIG. 1F-OA is the corresponding loadings scatter plot (p2 vs. p1) for the PCA shown in FIG. 1E-OA.

FIG. 2A-OA shows a section of the variable importance plot (VIP) derived from the PLS-DA model described in FIG. 1E-OA.

FIG. 2B-OA shows a section of the regression coefficient plot derived from the PLS-DA model described in FIG. 1E-OA.

FIG. 3-OA is a γ-predicted scatter plot for a PLS-DA model calculated using ˜85% of the control (triangles, ▴) and osteoporosis (circles, ●) samples, which was then used to predict the presence of disease in the remaining 15% of samples (squares, ▪) (the validation set).

### DETAILED DESCRIPTION OF THE INVENTION

Introduction

The inventors have developed novel methods (which employ multivariate statistical analysis and pattern recognition (PR) techniques, and optionally data filtering techniques) of analysing data (e.g., NMR spectra) from a test population which yield accurate mathematical models which may subsequently be used to classify a test sample or subject, and/or in diagnosis.

An NMR spectrum provides a fingerprint or profile for the sample to which it pertains. Such spectra represent a measure of all NMR detectable species present in the sample (rather than a select few) and also, to some extent, interactions between these species. As such, these spectra are characterised by a high data density which, heretofore, has not been fully exploited.

The methods described herein facilitate the analysis of such spectra, and the subsequent use of the results of that analysis to classify test spectra (and therefore the associated samples and subjects, if applicable) according to one or more distinguishing criteria, at a discrimination level never before achieved.

These methods find particular application in the field of medicine. For example, analysis of NMR spectra for samples taken from a population characterised by a certain condition yields a mathematical model which can be used to classify an NMR spectrum for a sample from a test subject as positive (also having the condition) or negative (not having the condition) with a high degree of confidence.

In effect, these methods facilitate the identification of the particular combination of amounts of (e.g., endogenous) species which are invariably associated with the presence of the condition. These combinations (patterns), which typically comprise many (often small) uncorrelated variances which together are diagnostic, are encoded within the high data density of the NMR spectra. The methods described herein permit their identification and subsequent use for classification.

However, it must be stressed that metabonomic analysis based on NMR spectra is much more powerful than simply using a high technology analytical tool (the NMR spectrometer) to measure the levels of known metabolites. That is, the methods described herein are distinct from methods which simply carry out multiple independent measures of discrete chemical entitities (e.g., LDL cholesterol concentration).

For example, considering the variance in NMR spectral intensity (total peak intensity) in any particular defined chemical shift region (known as a bucket or bin), a part of that variance may be associated with a given molecule (a biomarker), the level of which varies consistently as a result of the condition under study. The remainder of the variance may be due to differences in the levels of other molecules which give peaks in that integral region but which are unrelated to the condition under study (e.g., individual to individual differences such as dietary factors, age, gender, etc.).

The methods described herein, which employ pattern recognition techniques, permit identification of that NMR peak intensity which is related to the condition under study, even though only a small part of the variance in a spectral region (bucket) may be related to the condition under study. The identification power is enhanced by the application of data filtering techniques (e.g., orthogonal signal correction, OSC) which can lower the influence of buckets with variance unrelated to the condition of interest. Actual identification of the molecular biomarkers contributing to significant buckets is carried out by reexamination of the original NMR spectra by NMR experts, and could involve additional NMR spectroscopic experiments such as 2-dimensional NMR spectroscopy; separation of putative substances and their identification using HPLC-NMR-MS; addition of authentic substance to the sample and re-measuring the NMR spectrum, checking for coincidence of NMR peaks; etc.

For example, in NMR spectra of blood plasma, in the region around δ 1.2-1.3, a number of peaks appear, all of which will contribute to the intensity in those buckets labelled δ 1.30 (e.g., the chemical shift region δ 1.32-1.28), δ 1.26 (e.g., the region δ 1.28-1.24), and δ 1.22 (e.g., the region δ 1.24-1.20). Given the bucket width of 0.04 ppm (i.e., 24 Hz at 600 MHz), the wings of the lorentzian lines of the NMR resonances will have contributions in most or all of these buckets even though the peak maximum appears in a single bucket The two main broad NMR peak envelopes in this region of the spectrum have been assigned to the long chain methylene groups of the fatty acyl chains of lipoproteins, and in addition there are a number of small molecule metabolites which have NMR resonances in this region, some of which have been assigned. See, e.g., Nicholson et al, 1995. These include the methyl resonances of lactate (a doublet at δ 1.33), threonine (a doublet at δ 1.32), fucose (a doublet at δ 1.31), in some cases 3-hydroxybutyrate (a doublet at δ 1.20) and part of the methylene resonance of isoleucine (a multiplet at δ 1.28). The two overlapping lipoprotein peaks have been assigned as mainly VLDL at δ 1.29 and mainly LDL at δ 1.25. However both of these signals are asymmetric in appearance and are comprised of a number of overlapping resonances. By examination of the ¹H NMR spectra of individual lipoprotein fractions, it has been possible to use mathematical deconvolution techniques to show that this composite envelope in the δ 1.3-1.2 region is comprised of two bands from VLDL, 3 bands from LDL and 2 bands from HDL. See, e.g., M. Ala-Korpela, Progress in NMR Spectroscopy, 27, 475-554 (1995)). In fact, the inventors have shown that the variance in the spectral intensity in the bucket at δ 1.30 is only weakly correlated with the LDL level measured independently for a panel of 100 patients. The correlation coefficient (r) between the level of LDL as measured by a conventional method and the bucket intensity at δ 1.30 in the NMR spectra of the same samples, is only 0.45. Therefore, the changes in the concentration of LDL over the samples in this panel of 100 patients only accounts for about 20% of the variance in this bucket intensity, since variance is proportional to r². Thus the variance in the intensity in the δ 1.30 bucket, over the sample population, contains much more information than solely the variance in the LDL concentration. The methods the present invention permit the determination and exploitation of such of the additional, until now hidden, information.

Furthermore, the methods can be applied to achieve classification into multiple categories on the basis of a single dataset (e.g., an NMR spectrum for a single sample). Due to the very high data density of the input dataset, the analysis method can separately (i.e., in parallel) or sequentially (i.e., in series) perform multiple classifications. For example, a single blood sample could be used to determine (e.g., diagnose) the presence or absence of several, or indeed, many, (e.g., unrelated) conditions or diseases.

Thus, one aspect of the present invention pertains to improved methods for the analysis of chemical, biochemical, and biological data, for example spectra, for example, nuclear magnetic resonance (NMR) and other types of spectra.

Bone Disorders: Osteoarthritis

These techniques have been applied to the analysis of blood serum in the context of osteoarthritis. For example, the metabonomic analysis can distinguish between individuals with and without osteoarthritis. Novel diagnostic biomarkers for osteoarthritis have been identified, and associated methods for diagnosis have been described.

Methods of Classifying, Diagnosing

One aspect of the present invention pertains to a method of classifying a sample, as described herein.

One aspect of the present invention pertains to a method of classifying a subject by classifying a sample from said subject, wherein said method of classifying a sample is as described herein.

One aspect of the present invention pertains to a method of diagnosing a subject by classifying a sample from said subject, wherein said method of classifying a sample is as described herein.

Classifying a Sample: BY NMR Spectral Intensity

One aspect of the present invention pertains to a method of classifying a sample, said method comprising the step of relating NMR spectral intensity at one or more predetermined diagnostic spectral windows for said sample with a predetermined condition.

One aspect of the present invention pertains to a method of classifying a sample from a subject, said method comprising the step of relating NMR spectral intensity at one or more predetermined diagnostic spectral windows for said sample with a predetermined condition of said subject.

One aspect of the present invention pertains to a method of classifying a sample, said method comprising the step of relating NMR spectral intensity at one or more predetermined diagnostic spectral windows for said sample with the presence or absence of a predetermined condition.

One aspect of the present invention pertains to a method of classifying a sample from a subject, said method comprising the step of relating NMR spectral intensity at one or more predetermined diagnostic spectral windows for said sample with the presence or absence of a predetermined condition of said subject.

One aspect of the present invention pertains to a method of classifying a sample, said method comprising the step of relating a modulation of NMR spectral intensity, relative to a control value, at one or more predetermined diagnostic spectral windows for said sample with a predetermined condition.

One aspect of the present invention pertains to a method of classifying a sample from a subject, said method comprising the step of relating a modulation of NMR spectral intensity, relative to a control value, at one or more predetermined diagnostic spectral windows for said sample with a predetermined condition of said subject.

One aspect of the present invention pertains to a method of classifying a sample, said method comprising the step of relating a modulation of NMR spectral intensity, relative to a control value, at one or more predetermined diagnostic spectral windows for said sample with the presence or absence of a predetermined condition.

One aspect of the present invention pertains to a method of classifying a sample from a subject, said method comprising the step of relating a modulation of NMR spectral intensity, relative to a control value, at one or more predetermined diagnostic spectral windows for said sample with the presence or absence of a predetermined condition of said subject.

Classifying a Subject: By NMR Spectral Intensity

One aspect of the present invention pertains to a method of classifying a subject, said method comprising the step of relating NMR spectral intensity at one or more predetermined diagnostic spectral windows for a sample from said subject with a predetermined condition of said subject.

One aspect of the present invention pertains to a method of classifying a subject, said method comprising the step of relating NMR spectral intensity at one or more predetermined diagnostic spectral windows for a sample from said subject with the presence or absence of a predetermined condition of said subject.

One aspect of the present invention pertains to a method of classifying a subject, said method comprising the step of relating a modulation of NMR spectral intensity, relative to a control value, at one or more predetermined diagnostic spectral windows for a sample from said subject with a predetermined condition of said subject.

One aspect of the present invention pertains to a method of classifying a subject, said method comprising the step of relating a modulation of NMR spectral intensity, relative to a control value, at one or more predetermined diagnostic spectral windows for a sample from said subject with the presence or absence of a predetermined condition of said subject.

Diagnosing a Subject: By NMR Spectral Intensity

One aspect of the present invention pertains to a method of diagnosing a predetermined condition of a subject, said method comprising the step of relating NMR spectral intensity at one or more predetermined diagnostic spectral windows for a sample from said subject with said predetermined condition of said subject.

One aspect of the present invention pertains to a method of diagnosing a predetermined condition of a subject, said method comprising the step of relating NMR spectral intensity at one or more predetermined diagnostic spectral windows for a sample from said subject with the presence or absence of said predetermined condition of said subject.

One aspect of the present invention pertains to a method of diagnosing a predetermined condition of a subject, said method comprising the step of relating a modulation of NMR spectral intensity, relative to a control value, at one or more predetermined diagnostic spectral windows for a sample from said subject with said predetermined condition of said subject.

One aspect of the present invention pertains to a method of diagnosing a predetermined condition of a subject, said method comprising the step of relating a modulation of NMR spectral intensity, relative to a control value, at one or more predetermined diagnostic spectral windows for a sample from said subject with the presence or absence of said predetermined condition of said subject.

Classifying a Sample: By Amount of Diagnostic Species

One aspect of the present invention pertains to a method of classifying a sample, said method comprising the step of relating the amount of, or relative amount of one or more diagnostic species present in said sample with a predetermined condition.

One aspect of the present invention pertains to a method of classifying a sample from a subject, said method comprising the step of relating the amount of, or relative amount of one or more diagnostic species present in said sample with a predetermined condition of said subject.

One aspect of the present invention pertains to a method of classifying a sample, said method comprising the step of relating the amount of, or relative amount of one or more diagnostic species present in said sample with the presence or absence of a predetermined condition.

One aspect of the present invention pertains to a method of classifying a sample from a subject, said method comprising the step of relating the amount of, or the relative amount of, one or more diagnostic species present in said sample with the presence or absence of a predetermined condition of said subject.

One aspect of the present invention pertains to a method of classifying a sample, said method comprising the step of relating a modulation of the amount of, or relative amount of one or more diagnostic species present in said sample, as compared to a control sample, with a predetermined condition.

One aspect of the present invention pertains to a method of classifying a sample from a subject, said method comprising the step of relating a modulation of the amount of, or relative amount of one or more diagnostic species present in said sample, as compared to a control sample, with a predetermined condition of said subject.

One aspect of the present invention pertains to a method of classifying a sample, said method comprising the step of relating a modulation of the amount of, or relative amount of one or more diagnostic species present in said sample, as compared to a control sample, with the presence or absence of a predetermined condition.

One aspect of the present invention pertains to a method of classifying a sample from a subject, said method comprising the step of relating a modulation of the amount of, or relative amount of one or more diagnostic species present in said sample, as compared to a control sample, with the presence or absence of a predetermined condition of said subject.

Classifying a Subject: By Amount of Diagnostic Species

One aspect of the present invention pertains to a method of classifying a subject, said method comprising the step of relating the amount of, or relative amount of one or more diagnostic species present in a sample from said subject with a predetermined condition of said subject.

One aspect of the present invention pertains to a method of classifying a subject, said method comprising the step of relating the amount of, or relative amount of one or more diagnostic species present in a sample from said subject with the presence or absence of a predetermined condition of said subject.

One aspect of the present invention pertains to a method of classifying a subject, said method comprising the step of relating a modulation of the amount of, or relative amount of one or more diagnostic species present in a sample from said subject, as compared to a control sample, with a predetermined condition of said subject.

One aspect of the present invention pertains to a method of classifying a subject, said method comprising the step of relating a modulation of the amount of, or relative amount of one or more diagnostic species present in a sample from said subject, as compared to a control sample, with the presence or absence of a predetermined condition of said subject.

Diagnosing a Subject: By Amount of Diagnostic Species

One aspect of the present invention pertains to a method of diagnosing a predetermined condition of a subject, said method comprising the step of relating the amount of, or relative amount of one or more diagnostic species present in a sample from said subject with said predetermined condition of said subject.

One aspect of the present invention pertains to a method of diagnosing a predetermined condition of a subject, said method comprising the step of relating the amount of, or relative amount of one or more diagnostic species present in a sample from said subject with the presence or absence of said predetermined condition of said subject.

One aspect of the present invention pertains to a method of diagnosing a predetermined condition of a subject, said method comprising the step of relating a modulation of the amount of, or relative amount of one or more diagnostic species present in a sample from said subject, as compared to a control sample, with said predetermined condition of said subject.

One aspect of the present invention pertains to a method of diagnosing a predetermined condition of a subject, said method comprising the step of relating a modulation of the amount of, or relative amount of one or more diagnostic species present in a sample from said subject, as compared to a control sample, with the presence or absence of said predetermined condition of said subject.

Classifying a Sample: By Mathematical Modelling

One aspect of the present invention pertains to a method of classification, said method comprising the steps of

(a) forming a predictive mathematical model by applying a modelling method to modelling data;

(b) using said model to classify a test sample.

One aspect of the present invention pertains to a method of classifying a test sample, said method comprising the steps of:

(a) forming a predictive mathematical model by applying a modelling method to modelling data;

wherein said modelling data comprises a plurality of data sets for modelling samples of known class;

(b) using said model to classify said test sample as being a member of one of said known classes.

One aspect of the present invention pertains to a method of classifying a test sample, said method comprising the steps of:

(a) forming a predictive mathematical model by applying a modelling method to modelling data;

wherein said modelling data comprises at least one data set for-each of a plurality of modelling samples;

wherein said modelling samples define a class group consisting of a plurality of classes;

wherein each of said modelling samples is of a known class selected from said class group; and,

(b) using said model with a data set for said test sample to classify said test sample as being a member of one class selected from said class group.

One aspect of the present invention pertains to a method of classification, said method comprising the step of:

using a predictive mathematical model;

wherein said model is formed by applying a modelling method to modelling data;

to classify a test sample.

One aspect of the present invention pertains to a method of classifying a test sample, said method comprising the step of:

using a predictive mathematical model;

wherein said model is formed by applying a modelling method to modelling data;

wherein said modelling data comprises a plurality of data sets for modelling samples of known class;

to classify said test sample as being a member of one of said known classes.

One aspect of the present invention pertains to a method of classifying a test sample, said method comprising the step of:

using a predictive mathematical model;

wherein said model is formed by applying a modelling method to modelling data;

wherein said modelling data comprises at least one data set for each of a plurality of modelling samples;

wherein said modelling samples define a class group consisting of a plurality of classes;

wherein each of said modelling samples is of a known class selected from said class group;

with a data set for said test sample to classify said test sample as being a member of one class selected from said class group.

Classifying a Subject: By Mathematical Modelling

One aspect of the present invention pertains to a method of classification, said method comprising the steps of:

(a) forming a predictive mathematical model by applying a modelling method to modelling data;

(b) using said model to classify a subject.

One aspect of the present invention pertains to a method of classifying a subject, said method comprising the steps of:

(a) forming a predictive mathematical model by applying a modelling method to modelling data;

wherein said modelling data comprises a plurality of data sets for modelling samples of known class;

(b) using said model to classify a test sample from said subject as being a member of one of said known classes, and thereby classify said subject.

One aspect of the present invention pertains to a method of classifying a subject, said method comprising the steps of:

(a) forming a predictive mathematical model by applying a modelling method to modelling data;

wherein said modelling data comprises at least one data set for each of a plurality of modelling samples;

wherein said modelling samples define a class group consisting of a plurality of classes;

wherein each of said modelling samples is of a known class selected from said class group; and,

(b) using said model with a data set for a test sample from said subject to classify said test sample as being a member of one class selected from said class group, and thereby classify said subject.

One aspect of the present invention pertains to a method of classification, said method comprising the step of:

using a predictive mathematical model;

wherein said model is formed by applying a modelling method to modelling data;

to classify a subject.

One aspect of the present invention pertains to a method of classifying a subject, said method comprising the step of:

using a predictive mathematical model

wherein said model is formed by applying a modelling method to modelling data;

wherein said modelling data comprises a plurality of data sets for modelling samples of known class;

to classify a test sample from said subject as being a member of one of said known classes, and thereby classify said subject.

One aspect of the present invention pertains to a method of classifying a subject, said method comprising the step of:

using a predictive mathematical model,

wherein said model is formed by applying a modelling method to modelling data;

wherein said modelling data comprises at least one data set for each of a plurality of modelling samples;

wherein said modelling samples define a class group consisting of a plurality of classes;

wherein each of said modelling samples is of a known class selected from said class group;

with a data set for a test sample from said subject to classify said test sample as being a member of one class selected from said class group, and thereby classify said subject.

Diagnosing a Subject: By Mathematical Modelling

One aspect of the present invention pertains to a method of diagnosis, said method comprising the steps of:

(a) forming a predictive mathematical model by applying a modelling method to modelling data;

(b) using said model to diagnose a subject.

One aspect of the present invention pertains to a method of diagnosing a predetermined condition of a subject, said method comprising the steps of:

(a) forming a predictive mathematical model by applying a modelling method to modelling data;

wherein said modelling data comprises a plurality of data sets for modelling samples of known class;

(b) using said model to classify a test sample from said subject as being a member of one of said known classes, and thereby diagnose said subject.

One aspect of the present invention pertains to a method of diagnosing a predetermined condition of a subject, said method comprising the steps of:

(a) forming a predictive mathematical model by applying a modelling method to modelling data;

wherein said modelling data comprises at least one data set for each of a plurality of modelling samples;

wherein said modelling samples define a class group consisting of a plurality of classes;

wherein each of said modelling samples is of a known class selected from said class group; and,

(b) using said model with a data set for a test sample from said subject to classify said test sample as being a member of one class selected from said class group, and thereby diagnose said subject.

One aspect of the present invention pertains to a method of diagnosis, said method comprising the step of:

using a predictive mathematical model;

wherein said model is formed by applying a modelling method to modelling data;

to diagnose a subject.

One aspect of the present invention pertains to a method of diagnosing a predetermined condition of a subject, said method comprising the step of:

using a predictive mathematical model;

wherein said model is formed by applying a modelling method to modelling data;

wherein said modelling data comprises a plurality of data sets for modelling samples of known class;

to classify a test sample from said subject as being a member of one of said known classes, and thereby diagnose said subject.

One aspect of the present invention pertains to a method of diagnosing a predetermined condition of a subject, said method comprising the step of:

using a predictive mathematical model;

wherein said model is formed by applying a modelling method to modelling data;

wherein said modelling data comprises at least one data set for each of a plurality of modelling samples;

wherein said modelling samples define a class group consisting of a plurality of classes;

wherein each of said modelling samples is of a known class selected from said class group;

with a data set for a test sample from said subject to classify said test sample as being a member of one class selected from said class group, and thereby diagnose said subject.

### CERTAIN PREFERRED EMBODIMENTS

In one embodiment, said sample is a sample from a subject, and said predetermined condition is a predetermined condition of said subject.

In one embodiment, said test sample is a test sample from a subject, and said predetermined condition is a predetermined condition of said subject.

In one embodiment, said one or more predetermined diagnostic spectral windows are associated with one or more diagnostic species.

In one embodiment, said relating step involves the use of a predictive mathematical model; for example, as described herein.

The nature of a predictive mathematical model is determined primarily by the modelling method employed when forming that model.

In one embodiment, said modelling method is a multivariate statistical analysis modelling method.

In one embodiment, said modelling method is a multivariate statistical analysis modelling method which employs a pattern recognition method.

In one embodiment, said modelling method is, or employs PCA.

In one embodiment, said modelling method is, or employs PLS.

In one embodiment, said modelling method is, or employs PLS-DA.

In one embodiment, said modelling method includes a step of data filtering.

In one embodiment, said modelling method includes a step of orthogonal data filtering.

In one embodiment, said modelling method includes a step of OSC.

In one embodiment, said model takes account of one or more diagnostic species.

The precise details of the predictive mathematical model are determined primarily by the modelling data (e.g., modelling data sets).

In one embodiment, said modelling data comprise spectral data.

In one embodiment, said modelling data comprise both spectral data and non-spectral data (and is referred to as a “composite data”).

In one embodiment, said modelling data comprise NMR spectral data.

In one embodiment, said modelling data comprise both NMR spectral data and non-NMR spectral data.

In one embodiment, said NMR spectral data comprises ¹H NMR spectral data and/or ¹³C NMR spectral data.

In one embodiment, said NMR spectral data comprises ¹H NMR spectral data.

In one embodiment, said modelling data comprise spectra.

In one embodiment, said modelling data are spectra.

In one embodiment, said modelling data comprises a plurality of data sets for modelling samples of known class.

In one embodiment, said modelling data comprises at least one data set for each of a plurality of modelling samples.

In one embodiment, said modelling data comprises exactly one data set for each of a plurality of modelling samples.

In one embodiment, said using step is: using said model with a data set for said test sample to classify said test sample as being a member of one class selected from said class group.

In one embodiment, each of said data sets comprises spectral data.

In one embodiment, each of said data sets comprises both spectral data and non-spectral data (and is referred to as a “composite data set”).

In one embodiment, each of said data sets comprises NMR spectral data.

In one embodiment, each of said data sets comprises both NMR spectral data and non-NMR spectral data.

In one embodiment, said NMR spectral data comprises ¹H NMR spectral data and/or ¹³C NMR spectral data.

In one embodiment, said NMR spectral data comprises ¹H NMR spectral data.

In one embodiment, each of said data sets comprises a spectrum.

In one embodiment, each of said data sets comprises a ¹H NMR spectrum and/or ¹³C NMR spectrum.

In one embodiment, each of said data sets comprises a ¹H NMR spectrum.

In one embodiment, each of said data sets is a spectrum.

In one embodiment, each of said data sets is a ¹H NMR spectrum and/or ¹³C NMR spectrum.

In one embodiment, each of said data sets is a ¹H NMR spectrum.

In one embodiment, said non-spectral data is non-spectral clinical data.

In one embodiment, said non-NMR spectral data is non-spectral clinical data.

In one embodiment, said class group comprises classes associated with said predetermined condition (e.g., presence, absence, degree, etc.).

In one embodiment, said class group comprises exactly two classes.

In one embodiment, said class group comprises exactly two classes: presence of said predetermined condition; and absence of said predetermined condition.

Classification, Classifying, and Classes

As discussed above, many aspects of the present invention pertain to methods of classifying things, for example, a sample, a subject, etc. In such methods, the thing is classified, that is, it is associated with an outcome, or, more specifically, it is assigned membership to a particular class (i.e., it is assigned class membership), and is said “to be of,” “to belong to,” “to be a member of,” a particular class.

Classification is made (i.e., class membership is assigned) on the basis of diagnostic criteria. The step of considering such diagnostic criteria, and assigning class membership, is described by the word “relating,” for example, in the phrase “relating NMR spectral intensity at one or more predetermined diagnostic spectral windows for said sample (i.e., diagnostic criteria) with the presence or absence of a predetermined condition (i.e., class membership).”

For example, “presence of a predetermined condition” is one class, and “absence of a predetermined condition” is another class; in such cases, classification (i.e., assignment to one of these classes) is equivalent to diagnosis.

Samples

As discussed above, many aspects of the present invention pertain to methods which involve a sample, e.g., a particular sample under study (“study sample”).

In general, a sample may be in any suitable form. For methods which involve spectra obtained or recorded for a sample, the sample may be in any form which is compatible with the particular type of spectroscopy, and therefore may be, as appropriate, homogeneous or heterogeneous, comprising one or a combination of, for example, a gas, a liquid, a liquid crystal, a gel, and a solid.

Samples which originate from an organism (e.g., subject, patient) may be in vivo; that is, not removed from or separated from the organism. Thus, in one embodiment, said sample is an in vivo sample. For example, the sample may be circulating blood, which is “probed” in situ, in vivo, for example, using NMR methods.

Samples which originate from an organism may be ex vivo; that is, removed from or separated from the organism (e.g., an ex vivo blood sample, an ex vivo urine sample). Thus, in one embodiment, said sample is an ex vivo sample.

In one embodiment, said sample is an ex vivo blood or blood-derived sample.

In one embodiment, said sample is an ex vivo blood sample.

In one embodiment, said sample is an ex vivo plasma sample.

In one embodiment, said sample is an ex vivo serum sample.

In one embodiment, said sample is an ex vivo urine sample.

In one embodiment, said sample is removed from or separated from an/said organism, and is not returned to said organism (e.g., an ex vivo blood sample, an ex vivo urine sample).

In one embodiment, said sample is removed from or separated from an/said organism, and is returned to said organism (i.e., “in transit”) (e.g., as with dialysis methods). Thus, in one embodiment, said sample is an ex vivo in transit sample.

Examples of samples include:

a whole organism (living or dead, e.g., a living human);

a part or parts of an organism (e.g., a tissue sample, an organ);

a pathological tissue such as a tumour;

a tissue homogenate (e.g. a liver microsome fraction);

an extract prepared from a organism or a part of an organism (e.g., a tissue sample extract, such as perchloric acid extract);

an infusion prepared from a organism or a part of an organism (e.g., tea, Chinese traditional herbal medicines);

an in vitro tissue such as a spheroid;

a suspension of a particular cell type (e.g. hepatocytes);

an excretion, secretion, or emission from an organism (especially a fluid);

material which is administered and collected (e.g., dialysis fluid);

material which develops as a function of pathology (e.g., a cyst, blisters); and, supernatant from a cell culture.

Examples of fluid samples include, for example, blood plasma, blood serum, whole blood, urine, (gall bladder) bile, cerebrospinal fluid, milk, saliva, mucus, sweat, gastric juice, pancreatic juice, seminal fluid, prostatic fluid, seminal vesicle fluid, seminal plasma, amniotic fluid, foetal fluid, follicular fluid, synovial fluid, aqueous humour, ascite fluid, cystic fluid, blister fluid, and cell suspensions; and extracts thereof.

Examples of tissue samples include liver, kidney, prostate, brain, gut, blood, blood cells, skeletal muscle, heart muscle, lymphoid, bone, cartilage, and reproductive tissues.

Still other examples of samples include air (e.g., exhaust), water (e.g., seawater, groundwater, wastewater, e.g., from factories), liquids from the food industry (e.g. juices, wines, beers, other alcoholic drinks, tea, milk), solid-like food samples (e.g. chocolate, pastes, fruit peel, fruit and vegetable flesh such as banana, leaves, meats, whether cooked or raw, etc.).

A few preferred samples are discussed below.

Blood, Plasma, Serum

Blood is the fluid that circulates in the blood vessels of the body, that is, the fluid that is circulated through the heart, arteries, veins, and capillaries. The function of the blood and the circulation is to service the needs of other tissues: to transport oxygen and nutrients to the tissues, to transport carbon dioxide and various metabolic waste products away, to conduct hormones from one part of the body to another, and in general to maintain an appropriate environment in all tissue fluids for optimal survival and function of the cells.

Blood consists of a liquid component, plasma, and a solid component, cells and formed elements (e.g., erythrocytes, leukocytes, and platelets), suspended within it. Erythrocytes, or red blood cells account for about 99.9% of the cells suspended in human blood. They contain hemoglobin which is involved in the transport of oxygen and carbon dioxide. Leukocytes, or white blood cells, account for about 0.1% of the cells suspended in human blood. They play a role in the body's defense mechanism and repair mechanism, and may be classified as agranular or granular. Agranular leukocytes include monocytes and small, medium and large lymphocytes, with small lymphocytes accounting for about 20-25% of the leukocytes in human blood. T cells and B cells are important examples of lymphocytes. Three classes of granular leukocytes are known, neutrophils, eosinophils, and basophils, with neutrophils accounting for about 60% of the leukocytes in human blood. Platelets (i.e., thrombocytes) are not cells but small spindle-shaped or rodlike bodies about 3 microns in length which occur in large numbers in circulating blood. Platelets play a major role in clot formation.

Plasma is the liquid component of blood. It serves as the primary medium for the transport of materials among cellular, tissue, and organ systems and their various external environments, and it is essential for the maintenance of normal hemostasis. One of the most important functions of many of the major tissue and organ systems is to maintain specific components of plasma within acceptable physiological limits.

Plasma is the residual fluid of blood which remains after removal of suspended cells and formed elements. Whole blood is typically processed to removed suspended cells and formed elements (e.g., by centrifugation) to yield blood plasma. Serum is the fluid which is obtained after blood has been allowed to clot and the clot removed. Blood serum may be obtained by forming a blood clot (e.g., optionally initiated by the addition of thrombin and calcium ion) and subsequently removing the clot (e.g., by centrifugation). Serum and plasma differ primarily in their content of fibrinogen and several components which are removed in the clotting process. Plasma may be effectively prevented from clotting by the addition of an anti-coagulant (e.g., sodium citrate, heparin, lithium heparin) to permit handling or storage. Plasma is composed primarily of water (approximately 90%), with approximately 7% proteins, 0.9% inorganic salts, and smaller amounts of carbohydrates, lipids, and organic salts.

The term “blood sample,” as used herein, pertains to a sample of whole blood.

The term “blood-derived sample,” as used herein, pertains to an ex vivo sample derived from the blood of the subject under study.

Examples of blood and blood-derived samples include, but are not limited to, whole blood (WB), blood plasma (including, e.g., fresh frozen plasma (FFP)), blood serum, blood fractions, plasma fractions, serum fractions, blood fractions comprising red blood cells (RBC), platelets (PLT), leukocytes, etc., and cell lysates including fractions thereof (for example, cells, such as red blood cells, white blood cells, etc., may be harvested and lysed to obtain a cell lysate).

Methods for obtaining, preparing, handling, and storing blood and blood-derived samples (e.g., plasma, serum) are well known in the art. Typically, blood is collected from subjects using conventional techniques (e.g., from the ante-cubital fossa), typically pre-prandially.

For use in the methods described herein, the method used to prepare the blood fraction (e.g., serum) should be reproduced as carefully as possible from one subject to the next. It is important that the same or similar procedure be used for all subjects. It may be preferable to prepare serum (as opposed to plasma or other blood fractions) for two reasons: (a) the preparation of serum is more reproducible from individual to individual than the preparation of plasma, and (b) the preparation of plasma requires the addition of anticoagulants (e.g., EDTA, citrate, or heparin) which will be visible in the NMR metabonomic profile and may reduce the data density available.

A typical method for the preparation of serum suitable for analysis by the methods described herein is as follows: 10 mL of blood is drawn from the antecubital fossa of an individual who had fasted overnight, using an 18 gauge butterfly needle. The blood is immediately dispensed into a polypropylene tube and allowed to clot at room temperature for 3 hours. The clotted blood is then subjected to centrifugation (e.g., 4,500×g for 5 minutes) and the serum supernatant removed to a clean tube. If necessary, the centrifugation step can be repeated to ensure the serum is efficiently separated from the clot. The serum supernatant may be analysed “fresh” or it may be stored frozen for later analysis.

A typical method for the preparation of plasma suitable for analysis by the methods described herein is as follows: High quality platelet-poor plasma is made by drawing the blood using a 19 gauge butterfly needle without the use of a tourniquet from the anetcubital fossa. The first 2 mL of blood drawn is discarded and the remainder is rapidly mixed and aliquoted into Diatube H anticoagulant tubes (Becton Dickinson). After gentle mixing by inversion the anticoagulated blood is cooled on ice for 15 minutes then subjected to centrifugation to pellet the cells and platelets (approximately 1,200×g for 15 minutes). The platelet poor plasma supernantant is carefully removed, drawing off the middle third of the supernatant and discarding the upper third (which may contain floating platelets) and the lower third which is too close to the readily disturbed platelet layer on the top of the cell pellet. The plasma may then be aliquoted and stored frozen at −20° C. or colder, and then thawed when required for assay.

Samples may be analysed immediately (“fresh”), or may be frozen and stored (e.g., at −80° C.) (“fresh frozen”) for future analysis. If frozen, samples are completely thawed prior to NMR analysis.

In one embodiment, said sample is a blood sample or a blood-derived sample.

In one embodiment, said sample is a blood sample.

In one embodiment, said sample is a blood plasma sample.

In one embodiment, said sample is a blood serum sample.

Urine

The composition of urine is complex and highly variable both between species and within species according to lifestyle. A wide range of organic acids and bases, simple sugars and polysaccharides, heterocycles, polyols, low molecular weight proteins and polypeptides are present together with inorganic species such as Na⁺, K⁺, Ca²⁺, Mg²⁺, HCO₃⁻, SO₄²⁻ and phosphates.

The term “urine,” as used herein, pertains to whole (or intact) urine, whether in vivo (e.g., foetal urine) or ex vivo, e.g., by excretion or catheterisation.

The term “urine-derived sample,” as used herein, pertains to an ex vivo sample derived from the urine of the subject under study (e.g., obtained by dilution, concentration, addition of additives, solvent- or solid-phase extraction, etc.). Analysis may be performed using, for example, fresh urine; urine which has been frozen and then thawed; urine which has been dried (e.g., freeze-dried) and then reconstituted, e.g., with water or D₂O.

Methods for the collection, handling, storage, and pre-analysis preparation of many classes of sample, especially biological samples (e.g., biofluids) are well known in the art. See, for example, Lindon et al., 1999.

In one embodiment, said sample is a urine sample or a urine-derived sample.

In one embodiment, said sample is a urine sample.

Organisms, Subjects, Patients

As discussed above, in many cases, samples are, or originate from, or are drawn or derived from, an organism (e.g., subject, patient). In such cases, the organism may be as defined below.

In one embodiment, the organism is a prokaryote (e.g., bacteria) or a eukaryote (e.g., protoctista, fungi, plants, animals).

In one embodiment, the organism is a prokaryote (e.g., bacteria) or a eukaryote (e.g., protoctista, fungi, plants, animals).

In one embodiment, the organism is a protoctista, an alga, or a protozoan.

In one embodiment, the organism is a plant, an angiosperm, a dicotyledon, a monocotyledon, a gymnosperm, a conifer, a ginkgo, a cycad, a fern, a horsetail, a clubmoss, a liverwort, or a moss.

In one embodiment, the organism is an animal.

In one embodiment, the organism is a chordate, an invertebrate, an echinoderm (e.g., starfish, sea urchins, brittlestars), an arthropod, an annelid (segmented worms) (e.g., earthworms, lugworms, leeches), a mollusk (cephalopods (e.g., squids, octopi), pelecypods (e.g., oysters, mussels, clams), gastropods (e.g., snails, slugs)), a nematode (round worms), a platyhelminthes (flatworms) (e.g., planarians, flukes, tapeworms), a cnidaria (e.g., jelly fish, sea anemones, corals), or a porifera (e.g., sponges).

In one embodiment, the organism is an arthropod, an insect (e.g., beetles, butterflies, moths), a chilopoda (centipedes), a diplopoda (millipedes), a crustacean (e.g., shrimps, crabs, lobsters), or an arachnid (e.g., spiders, scorpions, mites).

In one embodiment, the organism is a chordate, a vertebrate, a mammal, a bird, a reptile (e.g., snakes, lizards, crocodiles), an amphibian (e.g., frogs, toads), a bony fish (e.g., salmon, plaice, eel, lungfish), a cartilaginous fish (e.g., sharks, rays), or a jawless fish (e.g., lampreys, hagfish).

In one embodiment, the organism (e.g., subject, patient) is a mammal.

In one embodiment, the organism (e.g., subject, patient) is a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the organism may be any of its forms of development, for example, a spore, a seed, an egg, a larva, a pupa, or a foetus.

In one embodiment, the organism (e.g., subject, patient) is a human.

The subject (e.g., a human) may be characterised by one or more criteria, for example, sex, age (e.g., 40 years or more, 50 years or more, 60 years or more, etc.), ethnicity, medical history, lifestyle (e.g., smoker, non-smoker), hormonal status (e.g., pre-menopausal, post-menopausal), etc.

The term “population,” as used herein, refers to a group of organisms (e.g., subjects, patients). If desired, a population (e.g., of humans) may be selected according to one or more of the criteria listed above.

Conditions

As discussed above, many methods of the present invention involve assigning class membership, for example, to one of one or more classes, for example, to one of the two classes: (i) presence of a predetermined condition, or (ii) absence of a predetermined condition.

A condition is “predetermined” in the sense that it is the condition in respect to which the invention is practised; a condition is predetermined by a step of selecting a condition for considering, study, etc.

As used herein, the term “condition” relates to a state which is, in at least one respect, distinct from the state of normality, as determined by a suitable control population.

A condition may be pathological (e.g., a disease) or physiological (e.g., phenotype, genotype, fasting, water load, exercise, hormonal cycles, e.g., oestrus, etc.).

Included among conditions is the state of “at risk of” a condition, “predisposition towards a” condition, and the like, again as compared to the state of normality, as determined by a suitable control population. In this way, osteoporosis, at risk of osteoporosis, and predisposition towards osteoporosis are all conditions (and are also conditions associated with osteoporosis).

Where the condition is the state of “at risk of,” “predisposition towards,” and the like, a method of diagnosis may be considered to be a method of prognosis.

In this context, the phrases “at risk of,”, “predisposition towards,” and the like, indicate a probability of being classified/diagnosed (or being able to be classified/diagnosed) with the predetermined condition which is greater (e.g., 1.5×, 2×, 5×, 10×, etc.) than for the corresponding control. Often, a time period (e.g., within the next 5 years, 10 years, 20 years, etc.) is associated with the probability. For example, a subject who is 2× more likely to be diagnosed with the predetermined condition within the next 5 years, as compared to a suitable control, is “at risk of” that condition.

Included among conditions is the degree of a condition, for example, the progress or phase of a disease, or a recovery therefrom. For example, each of different states in the progress of a disease, or in the recovery from a disease, are themselves conditions. In this way, the degree of a condition may refer to how temporally advanced the condition is. Another example of a degree of a condition relates to its maximum severity, e.g., a disease can be classified as mild, moderate or severe). Yet another example of a degree of a condition relates to the nature of the condition (e.g., anatomical site, extent of tissue involvement, etc.).

Osteoarthritis

In the present invention, said predetermined condition is associated with osteoarthritis.

Osteoarthritis (OA) is the most prevalent type of arthritis, particularly in adults 65 years and older. OA is a chronic degenerative arthropathy that frequently leads to chronic pain and disability. With the aging of the population, this condition is becoming increasing prevalent and its treatment increasingly financially burdensome. Finding better treatments for OA is a major focus of research at this time. Reported incidence and prevalence rates of OA in specific joints vary widely, due to differences in the case definition of OA. OA may be defined by radiographic criteria alone (radiographic OA), by typical symptoms (symptomatic OA), or by both. Using radiographic criteria, the distal and proximal interphalangeal joints of the hand have been identified as the joints most commonly affected by OA, but they are the least likely to be symptomatic. In contrast, the knee and hip, which constitute the second and third most common locations of radiographic OA, respectively, are nearly always symptomatic. The first metatarsal phalangeal and carpometacarpal joints are also frequent sites of radiographic OA, while the shoulder, elbow, wrist and metacarpophalangeal joints rarely develop idiopathic OA

In demographic studies, age is the most consistently identified risk factor for OA, regardless of the joint being studied. Prevalence rates for both radiographic OA and, to a lesser extent, symptomatic OA rise steeply after age 50 in men and age 40 in women. Female gender is also a well-recognized risk factor for OA. Hand OA is particularly prevalent among women. In addition, polyarticular OA and isolated knee OA are slightly more common in women than men, while hip OA occurs more commonly in men. Women are more likely to report pain in all affected joints, including the hip, than men. Cohort studies have demonstrated a clear association of obesity with the development of radiographic knee OA in women and a weaker association with hip OA. Whether obesity is a risk factor for the development of hand OA remains controversial.

Occupation-related repetitive injury and physical trauma contribute to the development of secondary (non-idiopathic) OA, sometimes occurring in joints that are not affected by primary (idiopathic) OA, such as the metacarpophalangeal joints, wrists and ankles. Although the prevalence of knee OA is greater in adults who have engaged in occupations that require repetitive bending and strenuous activities, an association with regular, intense exercise remains controversial. While early studies in joggers failed to find a higher prevalence of OA of the knee in joggers compared to non-joggers, a recent study of the Framingham data base in elderly adults provided the first longitudinal association between high level of physical activity and incident knee OA Low-impact and recreational exercises are unlikely to constitute a risk factor for knee OA, and are likely to benefit the cardiovascular system. Prior menisectomy is a significant risk factor in men for the development of OA in the knee.

Signs and Symptoms of OA

OA is diagnosed by a triad of typical symptoms, physical findings and radiographic changes. The American College of Rheumatology has set forth classification criteria to aid in the identification of patients with symptomatic OA that include, but do not rely solely on, radiographic findings. Patients with early disease experience localized joint pain that worsens with activity and is relieved by rest, while those with severe disease may have pain at rest. Weight bearing joints may “lock” or “give way” due to internal derangement that is a consequence of advanced disease. Stiffness in the morning or following inactivity (“gel phenomenon”) rarely exceeds 30 minutes.

Physical findings in osteoarthritic joints include bony enlargement, crepitus, cool effusions, and decreased range of motion. Tenderness on palpation at the joint line and pain on passive motion are also common, although not unique to OA. Radiographic findings in OA include osteophyte formation, joint space narrowing, subchondral sclerosis and cysts. The presence of an osteophyte is the most specific radiographic marker for OA although it is indicative of relatively advanced disease.

Diagnosis

If a patient has the typical symptoms and radiographic features described above, the diagnosis of OA is relative straightforward and is unlikely to be confused with other entities. However, in less straightforward cases, other diagnoses should be considered. For example, periarticular pain that is not reproduced by passive motion or palpation of the joint should suggest an alternate etiology such as bursitis, tendonitis or periostitis. If the distribution of painful joints includes MCP, wrist, elbow, ankle or shoulder, OA is unlikely. Prolonged stiffness (greater than one hour) should raise suspicion for an inflammatory arthritis such as rheumatoid arthritis. Marked warmth and erythema in a joint suggests an infectious or microcrystalline etiology. Weight loss, fatigue, fever and loss of appetite suggest a systemic illness such as polymyalgia rheumatica, rheumatoid arthritis, lupus or sepsis or malignancy.

Radiographs are considered the “gold standard” test for the diagnosis of OA, but radiographic changes are evident only relatively late in the disease. The need is great for a sensitive and specific biological marker that would enable early diagnosis of OA, and monitoring of its progression. Routine laboratory studies, such as sedimentation rates and c-reactive protein, are not useful as markers for OA, although a recent study suggests that elevation of CRP predicts more rapidly progressive disease.

Several epitopes of cartilage components, however, have been described that offer some promise as markers of OA. For example, chondroitin sulfate epitope 846, normally expressed only in fetal and neonatal cartilage, has been observed in OA, but not normal adult, cartilage and synovial fluid. An epitope unique to type 11 collagen has been described in OA cartilage, and can be unmasked in vitro by exposing normal cartilage to MMPs. This epitope can be measured in blood and urine and may prove useful in diagnosing or monitoring OA progression. Elevated serum hyaluronan levels have also been shown by some to correlate with radiographic OA. The finding of elevated cartilage oligomeric protein (COMP) levels in synovial fluid after traumatic joint injury may portend development of OA in the injured joint. Other potential markers of OA have been listed but are either not easily accessible or lack the sensitivity and specificity required to consider them as potential OA markers.

Current treatment for OA is relatively limited. Because there are currently no pharmacological agents capable of retarding or preventing disease, treatment is predominantly focused on relief of pain, and maintenance of quality of life and functional independence.

Several studies have shown acetaminophen (paracetamol) to be superior to placebo and equivalent to nonsteroidal anti-inflammatory agents (NSAIDs) for the short-term management of OA pain. At present, acetaminophen (up to 4,000 mg/daily) is the recommended initial analgesic of choice for symptomatic OA. However, many patients eventually require NSAIDs or more potent analgesics to control pain.

The mechanism by which NSAIDs exert their anti-inflammatory and analgesic effects is via inhibition of the prostaglandin-generating enzyme, cyclooxygenase (COX). In addition to their inflammatory potential, prostaglandins also contribute to important homeostatic functions, such as maintenance of the gastric lining, renal blood flow, and platelet aggregation. Reduction of prostaglandin levels in these organs can result in the well-recognized side effects of traditional non-selective NSAIDs—that is, gastric ulceration, renal insufficiency, and prolonged bleeding time. The elderly are at higher risk for these side effects. For example, adults over the age of 60 who are taking NSAIDs have a 4-5 fold higher risk of gastrointestinal bleeding or ulceration then their age-matched counterparts. Other risk factors for NSAID-induced GI bleed include prior peptic ulcer disease and concomitant steroid use. Potential renal toxicities of NSAIDs include azoternia, proteinura, and renal failure requiring hospitalization. Hematologic and cognitive abnormalities have also been reported with several NSAIDs. Therefore, in elderly patients, and those with a documented history of NSAID-induced ulcers, traditional non-selective NSAIDs should be used with caution, usually in lower dose and in conjunction with a proton pump inhibitor. Renal function should be monitored in the elderly. In addition, prophylactic treatment to reduce risk of gastrointestinal ulceration, perforation and bleeding is recommended in patents >60 years of age with: prior history of peptic ulcer disease; anticipated duration of therapy of >3 months; moderate to high dose of NSAIDs; and, concurrent corticosteroids. Misoprostol, at a dose of 200 mg four times daily, constitutes effective anti-ulcer prophylaxis but is often poorly tolerated due to diarrhea. Omeprazole, and other proton pump inhibitors, are also very effective anti-ulcer prophylactic agents, although cost can be limiting. The recent development of selective cyclooxygenase-2 (COX-2) inhibitors offers a new strategy for the management of pain and inflammation that is likely to be less toxic to the GI tract.

The development of selective COX-2 inhibitors has been an exciting advance in the management of pain and inflammation, as discussed above. If the safety profile of the specific COX-2 inhibitors is confirmed in additional long-term studies to be superior to non-selective COX inhibitors, they are likely to replace traditional NSAIDs in the management of arthritis and other painful, inflammatory conditions. It should be kept in mind, however, that no matter what their degree of selectivity, COX inhibitors (NSAIDs) do not alter the natural history of OA and a “disease modifying” agent is still critically needed.

Local analgesic therapies include topical capsaicin and methyl salicylate creams.

Occasionally in late stage disease, patients will require narcotic analgesics to control pain. Oral glucosamine and chondroitin sulfate have been shown (each individually) to have a mild to moderate analgesic effect in several double-blind, placebo-controlled studies.

Judicious use of intra-articular glucocorticoid injections is appropriate for OA patients who cannot tolerate, or whose pain is not well controlled by, oral analgesic and anti-inflammatory agents. Periarticular injections may effectively treat bursitis or tendonitis that can accompany OA. The need for four or more intra-articular injections suggests the need for orthopedic intervention.

Intraarticular injection of hyaluronate preparations has been demonstrated in several small clinical trials to reduce pain in OA of the knee. These injections are given in a series of 3 or 5 weekly injections (depending on the specific preparation) and may reduce pain for up to 6 months in some patients.

Non-pharmacological Management

Weight reduction in obese patients has been shown to significantly relieve pain, presumably by reducing biomechanical stress on weight bearing joints. Exercise has also been shown to be safe and beneficial in the management of OA. It has been suggested that joint loading and mobilization are essential for articular integrity. In addition, quadricep weakness, which develops early in OA, may contribute independently to progressive articular damage. Several studies in older adults with symptomatic knee OA have shown consistent improvements in physical performance, pain and self-reported disability after 3 months of aerobic or resistance exercise. Other studies have shown that resistive strengthening improves gait, strength and overall function. Low-impact activities, including water-resistive exercises or bicycle training, may enhance peripheral muscle tone and strength and cardiovascular endurance, without causing excessive force across, or injury, to joints. Studies of nursing home and community-dwelling elderly clearly demonstrate that one additional important benefit of exercise is a reduction in the number of falls.

Surgical Management

Patients in whom function and mobility remain compromised despite maximal medical therapy, and those in whom the joint is structurally unstable, should be considered for surgical intervention. Patients in whom pain has progressed to unacceptable levels—that is, pain at rest and/or nighttime pain—should also be considered as surgical candidates. Surgical options include arthroscopy, osteotomy and arthroplasty. Arthroscopic removal of intra-articular loose bodies and repair of degenerative menisci may be indicated in some patients with knee OA. Tibial osteotomy is an option for some patients who have a relatively small varus angulation (less than 10 degrees) and stable ligamentous support. Total knee arthroplasty is recommended for patients with more severe varus, or any valgus, deformity and ligamentous instability. Arthroplasty is also indicated for patients who have had ineffective pain relief following a tibial osteotomy, and for those with advanced hip OA. Patients who have not yet developed appreciable muscle weakness, generalized or cardiovascular deconditioning and who would medically withstand the stress of surgery are ideal surgical candidates. In contrast, full mobility and function may not be realistically expected in patients with significant cognitive impairment or symptomatic cardiopulmonary disease, since these conditions can impede post-operative rehabilitation.

Several questionnaires have been established as validated, reliable research instruments for assessing functional outcomes in patients with arthritis. These include the Lequesne index, the Western Ontario McMaster Arthritis scale (WOMAC), activities of daily living (ADL), etc. Several performance-based tests of function can be done rapidly and easily in the office, however, and may be more sensitive in predicting impending disability than direct questions about disability and impairment. Such measures include grip strength, a timed walk, and sequential chair-stands. These tests can provide the clinician with valuable information on the patient's current level of function, as well as serve longitudinally to assess decline in function.

Osteoarthritis is the most prevalent articular disease in the elderly. Disease markers that will detect early disease, and agents that will slow down or halt disease progression are critically needed. Current management should include safe and adequate pain relief using systemic and local therapies, and should include medical and rehabilitative interventions that limit functional deterioration.

Although epidemiological studies have promoted understanding of the risk factors that predispose to OA, the initiating events that trigger the disease are not yet understood.

Cartilage is a unique tissue with viscoelastic and compressive properties which are imparted by its extracellular matrix, composed predominantly of type 11 collagen and proteoglycans. Under normal conditions, this matrix is subjected to a dynamic remodeling process in which low levels of degradative and synthetic enzyme activities are balanced, such that the volume of cartilage is maintained. In OA cartilage, however, matrix degrading enzymes are overexpressed, shifting this balance in favor of net degradation, with resultant loss of collagen and proteoglycans from the matrix. Presumably in response to this loss, chondrocytes initially proliferate and synthesize enhanced amounts of proteoglycan and collagen molecules. As the disease progresses, however, reparative attempts are outmatched by progressive cartilage degradation. Fibrillation, erosion and cracking initially appear in the superficial layer of cartilage and progress over time to deeper layers, resulting eventually in large clinically observable erosions. OA, in simplistic terms, therefore, can be thought of as a process of progressive cartilage matrix degradation to which an ineffectual attempt at repair is made.

A critical question is whether OA is truly a disease or a natural consequence of aging. Several differences between aging cartilage and OA cartilage have been described, suggesting the former. For example, although denatured type II collagen is found in both normal aging and OA cartilage, it is more predominant in OA. In addition, OA and normal aging cartilage differ in the amount of water content and the in ratio of chondroitin-sulfate to keratin sulfate constituents. The expression of a chondroitin-sulfate epitope (epitope 846) in OA cartilage, that is otherwise only present in fetal and neonatal cartilage, provides further evidence that OA is a distinct pathologic process. A final but important distinction is that degradative enzyme activity is increased in OA, but not in normal aging cartilage.

The primary enzymes responsible for the degradation of cartilage are the matrix metalloproteinases (MMPs). These enzymes are secreted by both synovial cells and chondrocytes and are categorized into three general categories: a) collagenases; b) stromelysins; and, c) gelatinases. Under normal conditions, MMP synthesis and activation are tightly regulated at several levels. They are secreted as inactive proenzymes that require enzymatic cleavage in order to become activated. Once activated, MMPs become susceptible to the plasma-derived MMP inhibitor, alpha-2-macroglobulin, and to tissue inhibitors of MMPs (TIMPs) that are also secreted by synovial cells and chondrocytes. In OA, synthesis of MMPs is greatly enhanced and the available inhibitors are overwhelmed, resulting in net degradation. Interestingly, stromelysin can serve as an activator for its own proenzyme, as well as for procollagenase and prostromelysin, thus creating a positive feedback loop of proMMP activation in cartilage.

One candidate is interleukin-1 (IL-1). IL-1 is a potent pro-inflammatory cytokine that, in vitro, is capable of inducing chondrocytes and synovial cells to synthesize MMPs. Furthermore, IL-1 suppresses the synthesis of type II collagen and proteoglycans, and inhibits transforming growth factor-β stimulated chondrocyte proliferation. The presence of IL-1 RNA and protein have been confirmed in OA joints. Thus, IL-1 may not only actively promote cartilage degradation, but may also suppress attempts at repair, in OA. In addition to these effects, IL-1 induces nitric oxide production, chondrocyte apoptosis, and prostaglandin synthesis, which further contribute to cartilage deterioration. Under normal conditions, an endogenous IL-1 receptor antagonist regulates IL-1 activity. A relative excess of IL-1 and/or deficiency of the IL-1 receptor antagonist could conceivably result in the cartilage destruction that is characteristic of OA. It is likely that other cytokines or particulate material from damaged cartilage may also contribute to this inflammatory, degradative process.

Can Cartilage Repair Itself?

Growth factors are produced locally in cartilage and synovium and are likely to contribute to local cartilage remodeling by stimulating the de novo synthesis of collagen and proteoglycans. Transforming growth factor β (TGFβ) is the best characterized and most potent of the chondrocyte growth factors. Not only does TGFβ stimulate de novo matrix synthesis, but it also counteracts cartilage degradation by down regulating IL-1 receptor expression and by increasing IL-1 receptor antagonist release and TIMP expression. Insulin-like growth factor (IGF-1) and basic fibroblast growth factor (b-FGF) are also present in OA cartilage and likely to contribute to reparative attempts, although, as noted, degradation ultimately outstrips repair in OA cartilage.

One disease modifying strategy is to suppress the progressive degradation of cartilage that occurs in OA. To accomplish this, the ratio of MMP inhibitors to MMP enzymes must be shifted in favor of the former. This could be accomplished by enhancing articular levels of TIMP by recombinant gene therapy or by administration of exogenous TIMP. Studies of exogenous TIMP administration to animals with OA-like disease have had inconclusive results, however, perhaps due to ineffective penetration of this relatively high molecular weight protein into the cartilage matrix. An alternate approach, that has progressed more rapidly, is to develop oral inhibitors of MMPs. In fact, several synthetic small molecular weight inhibitors of MMPs have proven efficacious in animal models of arthritis and are entering Phase III clinical trials in humans. The antibiotic, tetracycline, and its semisynthetic derivatives, doxycycline and minocycline, have modest MMP inhibitory properties and have prompted investigations of these agents in the treatment of both OA and RA. Finally, inhibition of IL-1, via administration of a soluble IL-1 receptor or receptor antagonist, represents another rational strategy for suppressing MMP synthesis, and preliminary studies in RA are also promising.

Enhancing the repair of damaged cartilage constitutes another rational strategy for the treatment and/or prevention of OA. Administration of exogenous growth factors, such as IGF and bFGF, to stimulate chondrocyte proliferation and/or matrix synthesis has had beneficial effects in animals models of OA, and TGF-B has the added advantage of suppressing MMP synthesis. The same effect could also be achieved theoretically by transplanting healthy autologous chondrocytes that have been genetically engineered to over-express one or more of these growth factors site. However, because the area of cartilage loss in OA can be quite extensive and because older chondrocytes are metabolically less active than young chondrocytes, it is unclear that either autologous or heterologous transplants will be practical for the management of OA.

In summary, MMPs and pro-inflammatory cytokines (e.g., IL-1) appear to be important mediators of cartilage destruction in OA. Synthesis and secretion of growth factors and of inhibitors of MMPs and cytokines are apparently inadequate to counteract these degradative forces. Progressive cartilage degradation and OA result. New therapies, focused on reducing MMP activity and on stimulating matrix synthesis, are in development.

NMR Spectroscopy

As discussed above, many aspects of the present invention pertain to methods which employ NMR spectra, or data obtained or derived from NMR spectra.

The principal nucleus studied in biomedical NMR spectroscopy is the proton or ¹H nucleus. This is the most sensitive of all naturally occurring nuclei. The chemical shift range is about 10 ppm for organic molecules. In addition ¹³C NMR spectroscopy using either the naturally abundant 1.1% ¹³C nuclei or employing isotopic enrichment is useful for identifying metabolites. The ¹³C chemical shift range is about 200 ppm. Other nuclei find special application. These include ¹⁵N (in natural abundance or enriched), ¹⁹F for studies of drug metabolism, and ³¹P for studies of endogenous phosphate biochemistry either in vitro or in vivo.

In order to obtain an NMR spectrum, it is necessary to define a “pulse program”. At its simplest, this is application of a radio-frequency (RF) pulse followed by acquisition of a free induction decay (FID)—a time-dependent oscillating, decaying voltage which is digitised in an analog-digital converter (ADC). At equilibrium, the nuclear spins are present in a number of quantum states and the RF pulse disturbs this equilibrium. The FID is the result of the spins returning towards the equilibrium state. It is necessary to choose the length of the pulse (usually a few microseconds) to give the optimum response.

This, and other experimental parameters are chosen on the basis of knowledge and experience on the part of the spectroscopist. See, for example, T. D. W. Claridge, High-Resolution NMR Techniques in Organic Chemistry: A Practical Guide to Modern NMR for Chemists, Oxford University Press, 2000. These are based on the observation frequency to be used, the known properties of the nucleus under study (i.e., the expected chemical shift range will determine the spectral width, the desired peak resolution determines the number of data points, the relaxation times determine the recycle time between scans, etc.). The number of scans to be added is determined by the concentration of the analyte, the inherent sensitivity of the nucleus under study and its abundance (either natural or enhanced by isotopic enrichment).

After data acquisition, a number of possible manipulations are possible. The FID can be multiplied by a mathematical function to improve the signal-to-noise ratio or reduce the peak line widths. The expert operator has choice over such parameters. The FID is then often filled by a number of zeros and then subjected to Fourier transformation. After this conversion from time-dependent data to frequency dependent data, it is necessary to phase the spectrum so that all peaks appear upright—this is done using two parameters by visual inspection on screen (now automatic routines are available with reasonable success). At this point the spectrum baseline can be curved. To remedy this, one defines points in the spectrum where no peaks appear and these are taken to be baseline. Usually, a polynomial function is fitted to these points, but other methods are available, and this function subtracted from the spectrum to provide a flat baseline. This can also be done in an automatic fashion. Other manipulations are also possible. It is possible to extend the FID forwards or backwards by “linear prediction” to improve resolution or to remove so-called truncation artefacts which occur if data acquisition of a scan is stopped before the FID has decayed into the noise. All of these decisions are also applicable to 2- and 3-dimensional NMR spectroscopy.

An NMR spectrum consists of a series of digital data points with a y value (relating to signal strength) as a function of equally spaced x-values (frequency). These data point values run over the whole of the spectrum. Individual peaks in the spectrum are identified by the spectroscopist or automatically by software and the area under each peak is determined either by integration (summation of the y values of all points over the peak) or by curve fitting. A peak can be a single resonance or a multiplet of resonances corresponding to a single type of nucleus in a particular chemical environment (e.g., the two protons ortho to the carboxyl group in benzoic acid). Integration is also possible of the three dimensional peak volumes in 2-dimensional NMR spectra. The intensity of a peak in an NMR spectrum is proportional to the number of nuclei giving rise to that peak (if the experiment is conducted under conditions where each successive accumulated free induction decay (FID) is taken starting at equilibrium). Also, the relative intensity of peaks from different analytes in the same sample is proportional to the concentration of that analyte (again if equilibrium prevails at the start of each scan).

Thus, the term “NMR spectral intensity,” as used herein, pertains to some measure related to the NMR peak area, and may be absolute or relative. NMR spectral intensity may be, for example, a combination of a plurality of NMR spectral intensities, e.g., a linear combination of a plurality of NMR spectral intensities.

In the context of NMR spectral intensity, the term “NMR” refers to any type of NMR spectroscopy.

NMR spectroscopic techniques can be classified according to the number of frequency axes and these include 1D-, 2D-, and 3D-NMR. 1D spectra include, for example, single pulse; water-peak eliminated either by saturation or non-excitation; spin-echo, such as CPMG (i.e., edited on the basis of spin-spin relaxation); diffusion-edited, selective excitation of specific spectra regions. 2D spectra include for example J-resolved (JRES); 1H-1H correlation methods, such as NOESY, COSY, TOCSY and variants thereof; heteronuclear correlation including direct detection methods, such as HETCOR, and inverse-detected methods, such as 1H-13C HMQC, HSQC, HMBC. 3D spectra, include many variants, all of which are combinations of 2D methods, e.g. HMQC-TOCSY, NOESY-TOCSY, etc. All of these NMR spectroscopic techniques can also be combined with magic-angle-spinning (MAS) in order to study samples other than isotropic liquids, such as tissues, which are characterised by anisotropic composition.

Preferred nuclei include ¹H and ¹³C. Preferred techniques for use in the present invention include water-peak eliminated, spin-echo such as CPMG, diffusion edited, JRES, COSY, TOCSY, HMQC, HSQC, and HMBC.

NMR analysis (especially of biofluids) is carried out at as high a field strength as is practical, according to availability (very high field machines are not widespread), cost (a 600 MHz instrument costs about £500,000 but a shielded 800 MHz instrument can cost more than £3,500,000, depending on the nature of accessory equipment purchased), and ability to accommodate the physical size of the instrument. Maintenance/operational costs do not vary greatly and are small compared to the capital cost of the machine and the personnel costs.

Typically, the ¹H observation frequency is from about 200 MHz to about 900 MHz, more typically from about 400 MHz to about 900 MHz, yet more typically from about 500 MHz to about 750 MHz. ¹H observation frequencies of 500 and 600 MHz may be particularly preferred. Instruments with the following ¹H observation frequencies are/were commercially available: 200, 250, 270 (discontinued), 300, 360 (discontinued), 400, 500, 600, 700, 750, 800, and 900 MHz.

Higher frequencies are used to obtain better signal-to-noise ratio and for greater spectral dispersion of resonances. This gives a better chance of identifying the molecules giving rise to the peaks. The benefit is not linear because in addition to the better dispersion, the detailed spectral peaks can move from being “second-order”—where analysis by inspection is not possible, towards “first-order,” where i is. Both peak positions and intensities within multiplets change in a non-linear fashion as this progression occurs. Lower observation frequencies would be used where cost is an issue, but this is likely to lead to reduced effectiveness for classification and identification of biomarkers.

NMR Spectroscopy: Sample Preparation

NMR spectra can be measured in solid, liquid, liquid crystal or gas states over a range of temperatures from 120 K to 420 K and outside this range with specialised equipment. Typically, NMR analysis of biofluids is performed in the liquid state with a sample temperature of from about 274 K to about 328 K, but more typically from about 283 K to about 321 K. An example of a typical temperature is about 300 K.

Lower temperatures would be used to ensure that the biofluid did not suffer from any decomposition or show any effects of chemical or enzymatic reactions during the data acquisition. Higher temperatures may be used to improve detection of certain species. For example, for plasma or serum, lipoproteins undergo a series of phase changes as the temperature is increased; in particular, the low density lipoprotein (LDL) peak intensities are rather temperature dependent and the lines sharpen and broader more-difficult-to-detect components become visible as the lipoprotein becomes more “liquid.”

Typically, biofluid samples are diluted with solvent prior to NMR analysis. This is done for a variety of reasons, including: to lessen solution viscosity, to control the pH of the solution, and to allow addition of reagents and reference materials.

An example of a typical dilution solvent is a solution of 0.9% by weight of sodium chloride in D₂O. The D₂O lessens the overall concentration of H₂O and eases the technical requirements in the suppression of the solvent water NMR resonance, necessary for optimum detection of metabolite NMR signals. The deuterium nuclei of the D₂O also provides an NMR signal for locking the magnetic field enabling the exact co-registration of successive scans.

Depending on the available amount of the biofluid, typically, the dilution ratio is from about 1:50 to about 5:1 by volume, but more typically from about 1:20 to about 1:1 by volume. An example of a typical dilution ratio is 3:7 by volume (e.g., 150 μL sample, 350 μL solvent), typical for conventional 5 mm NMR tubes and for flow-injection NMR spectroscopy.

Typical sample volumes for NMR analysis are from about 50 μL (e.g., for microprobes) to about 2 mL. An example of a typical sample volume is about 500 μL.

NMR peak positions (chemical shifts) are measured relative to that of a known standard compound usually added directly to the sample. For biofluids such as urine this is commonly a partially deuterated form of TSP, i.e., 3-trimethylsilyl-[2,2,3,3-²H₄]-propionate sodium salt. For biofluids containing high levels of proteins, this substance is not suitable since it binds to proteins and shows a broadened NMR line. Added formate anion (e.g., as a salt) can be used in such cases as for blood plasma.

NMR Spectroscopy: Manipulation of NMR Spectra

NMR spectra are typically acquired, and subsequently, handled in digitised form. Conventional methods of spectral pre-processing of (digital) spectra are well known, and include, where applicable, signal averaging, Fourier transformation (and other transformation methods), phase correction, baseline correction, smoothing, and the like (see, for example, Lindon et al., 1980).

Modern spectroscopic methods often permit the collection of high or very high resolution spectra. In digital form, even a simple spectrum (e.g., signal versus spectroscopic parameter) may have many thousands, if not tens of thousands of data points. It is often desirable to reduce or compress the data to give fewer data points, for both practical computing methods and also to effect some degree of signal averaging to compensate for physical effects, such as pH variation, compartmentalisation, and the like. The resulting data may be referred to as “spectral data.”

For example, a typical ¹H NMR spectrum is recorded as signal intensity versus chemical shift (δ) which ranges from about δ 0 to δ 10. At a typical chemical shift resolution of about δ 10⁻⁴-10⁻³ ppm, the spectrum in digital form comprises about 10,000 to 100,000 data points. As discussed above, it is often desirable to compress this data, for example, by a factor of about 10 to 100, to about 1000 data points.

For example, in one approach, the chemical shift axis, δ, is “segmented” into “buckets” or “bins” of a specific length. For a 1-D ¹H NMR spectrum which spans the range from δ 0 to δ 10, using a bucket length, Δδ, of 0.04 yields 250 buckets, for example, δ 10.0-9.96, δ 9.96-9.92, δ 9.92-9.88, etc., usually reported by their midpoint, for example, δ 9.98, δ 9.94, δ 9.90, etc. The signal intensity within a given bucket may be averaged or integrated, and the resulting value reported. In this way, a spectrum with, for example, 100,000 original data points can be compressed to an equivalent spectrum with, for example, 250 data points.

A similar approach can be applied to 2-D spectra, 3-D spectra, and the like. For 2-D spectra, the “bucket” approach may be extended to a “patch.” For 3-D spectra, the “bucket” approach may be extended to a “volume.” For example, a 2-D ¹H NMR. spectrum which spans the range from δ 0 to δ 10 on both axes, using a patch of Δδ 0.1×Δδ 0.1 yields 10,000 patches. In this way, a spectrum with perhaps 10⁸ original data points can be compressed to an equivalent spectrum of 10⁴ data points.

In this context, the equivalent spectrum may be referred to as “a spectral data set,” “a data set comprising spectral data,” etc.

Software for such processing of NMR spectra, for example AMIX (Analysis of MIXture, V 2.5, Bruker Analytik, Rheinstetten, Germany) is commercially available.

Often, certain spectral regions carry no real diagnostic information, or carry conflicting biochemical information, and it is often useful to remove these “redundant” regions before performing detailed analysis. In the simplest approach, the data points are deleted. In another simple approach, the data in the redundant regions are replaced with zero values.

For example, due to the dynamic range problem with water in comparison with other molecules, the water resonance (around δ 4.7) is suppressed. However, small variations in water suppression remain, and these variations can undesirably complicate analysis. Similarly, variations in water suppression may also affect the urea signal (around δ 6.0), by cross saturation. Therefore, it is often useful to delete certain spectral regions, for example, from about δ 4.5 to 6.0 (e.g., δ 4.52 to 6.00).

In general, NMR data is handled as a data matrix. Typically, each row in the matrix corresponds to an individual sample (often referred to as a “data vector”), and the entries in the columns are, for example, spectral intensity of a particular data point, at a particular δ or Δδ (often referred to as “descriptors”).

It is often useful to pre-process data, for example, by addressing missing data, translation, scaling, weighting, etc.

Multivariate projection methods, such as principal component analysis (PCA) and partial least squares analysis (PLS), are so-called scaling sensitive methods. By using prior knowledge and experience about the type of data studied, the quality of the data prior to multivariate modelling can be enhanced by scaling and/or weighting. Adequate scaling and/or weighting can reveal the important and interesting variation hidden within in the data, and therefore make subsequent multivariate modelling more efficient. Scaling and weighting may be used to place the data in the correct metric, based on knowledge and experience of the studied system, and therefore reveal patterns already inherently present in the data.

If at all possible, missing data, for example, gaps in column values, should be avoided. However, if necessary, such missing data may replaced or “filled” with, for example, the mean value of a column (“mean fill”); a random value (“random fill”); or a value based on a principal component analysis (“principal component fill”). Each of these different approaches will have a different effect on subsequent PR analysis.

“Translation” of the descriptor coordinate axes can be useful. Examples of such translation include normalisation and mean centring.

“Normalisation” may be used to remove sample-to-sample variation. Many normalisation approaches are possible, and they can often be applied at any of several points in the analysis. Usually, normalisation is applied after redundant spectral regions have been removed. In one approach, each spectrum is normalised (scaled) by a factor of 1/A, where A is the sum of the absolute values of all of the descriptors for that spectrum. In this way, each data vector has the same length, specifically, 1. For example, if the sum of the absolute values of intensities for each bucket in a particular spectrum is 1067, then the intensity for each bucket for this particular spectrum is scaled by 1/1067.

“Mean centring” may be used to simplify interpretation. Usually, for each descriptor, the average value of that descriptor for all samples is subtracted. In this way, the mean of a descriptor coincides with the origin, and all descriptors are “centred” at zero. For example, if the average intensity at δ 10.0-9.96, for all spectra, is 1.2 units, then the intensity at δ 10.0-9.96, for all spectra, is reduced by 1.2 units.

In “unit variance scaling,” data can be scaled to equal variance. Usually, the value of each descriptor is scaled by 1/StDev, where StDev is the standard deviation for that descriptor for all samples. For example, if the standard deviation at δ 10.0-9.96, for all spectra, is 2.5 units, then the intensity at δ 10.0-9.96, for all spectra, is scaled by 1/2.5 or 0.4. Unit variance scaling may be used to reduce the impact of “noisy” data. For example, some metabolites in biofluids show a strong degree of physiological variation (e.g., diurnal variation, dietary-related variation) that is unrelated to any pathophysiological process. Without unit variance scaling, these noisy metabolites may dominate subsequent analysis.

“Pareto scaling” is, in some sense, intermediate between mean centering and unit variance scaling. In effect, smaller peaks in the spectra can influence the model to a higher degree than for the mean centered case. Also, the loadings are, in general, more interpretable than for unit variance based models. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a-positive skew and/or when data spans a large range, e.g., several orders of magnitude. Usually, for each descriptor, the value is replaced by the logarithm of that value. For example, the intensity at δ 10.09.96 is replaced the logarithm of the intensity at δ 10.0-9.96, for all spectra.

In “equal range scaling,” each descriptor is divided by the range of that descriptor for all samples. In this way, all descriptors have the same range, that is, 1. For example, if, at δ 10.0-9.96, for all spectra, the largest value is 87 units and the smallest value is 1, then the range is 86 units, and the intensity at δ 10.0-9.96, for all spectra, is divided by 86 units. However, this method is sensitive to presence of outlier points.

In “autoscaling,” each data vector is mean centred and unit variance scaled. This technique is a very useful because each descriptor is then weighted equally and, in the case of NMR descriptors, large and small peaks are treated with equal emphasis. This can be important for metabolites present at very low, but still detectable, levels.

Several supervised methods of scaling data are also known. Some of these can provide a measure of the ability of a parameter (e.g., a descriptor) to discriminate between classes, and can be used to improve classification by stretching a separation.

For example, in “variance weighting,” the variance weight of a single parameter (e.g., a descriptor) is calculated as the ratio of the inter-class variances to the sum of the intra-class variances. A large value means that this variable is discriminating between the classes. For example, if the samples are known to fall into two classes (e.g., a training set), it is possible to examine the mean and variance of each descriptor. If a descriptor has very different mean values and a small variance, then it will be good at separating the classes.

“Feature weighting” is a more general description of variance weighting, where not only the mean and standard deviation of each descriptor is calculated, but other well known weighting factors, such as the Fisher weight, are used.

Multivariate Statistical Analysis

As discussed above, multivariate statistics analysis methods, including pattern recognition methods, are often the most convenient and efficient way to analyse complex data, such as NMR spectra.

For example, such analysis methods may be used to identify, for example diagnostic spectral windows and/or diagnostic species, for a particular condition under study.

Also, such analysis methods may be used to form a predictive model, and then use that model to classify test data. For example, one convenient and particularly effective method of classification employs multivariate statistical analysis modelling, first to form a model (a “predictive mathematical model”) using data (“modelling data”) from samples of known class (e.g., from subjects known to have, or not have, a particular condition), and second to classify an unknown sample (e.g., “test data”), as having, or not having, that condition.

Examples of pattern recognition methods include, but are not limited to, Principal Component Analysis (PCA) and Partial Least Squares-Discriminant Analysis (PLS-DA).

PCA is a bilinear decomposition method used for overviewing “clusters” within multivariate data. The data are represented in K-dimensional space (where K is equal to the number of variables) and reduced to a few principal components (or latent variables) which describe the maximum variation within the data, independent of any knowledge of class membership (i.e., “unsupervised”). The principal components are displayed as a set of “scores” (t) which highlight clustering, trends, or outliers, and a set of “loadings” (p) which highlight the influence of input variables on t. See, for example, Kowalski et al., 1986).

The PCA decomposition can be described by the following equation:

X=TP′+E

where T is the set of scores explaining the systematic variation between the observations in X and P is the set of loadings explaining the between variable variation and provides the explanation to clusters, trends, and outliers in the score space. The non-systematic part of the variation not explained by the model forms the residuals, E.

PLS-DA is a supervised multivariate method yielding latent variables describing maximum separation between known classes of samples. PLS-DA is based on PLS which is the regression extension of the PCA method explained earlier. When PCA works to explain maximum variation between the studied samples PLS-DA suffices to explain maximum separation between known classes of samples in the data (X). This is done by a PLS regression against a “dummy vector or matrix” (Y) carrying the class separating information. The calculated PLS components will thereby be more focused on describing the variation separating the classes in X if this information is present in the data. From an interpretation point of view all the features of PLS can be used, which means that the variation can be interpreted in terms of scores (t,u), loadings (p,c), PLS weights (w) and regression coefficients (b). The fact that a regression is carried out against a known class separation means that the PLS-DA is a supervised method and that the class membership has to be known prior to the actual modelling. Once a model is calculated and validated it can be used for prediction of class membership for “new” unknown samples. Judgement of class membership is done on basis of predicted class membership (Ypred), predicted scores (tpred) and predicted residuals (DmodXpred) using statistical significance limits for the decision. See, for example, Sjostrom et al., 1986; Stahle et al., 1987.

In PLS, the variation between the objects in X is described by the X-scores, T, and the variation in the Y-block regressed against is described in the Y-scores, U. In PLS-DA the Y-block is a “dummy vector or matrix” describing the class membership of each observation. Basically, what PLS does is to maximize the covariance between T and U. For each component, a PLS weight vector, w, is calculated, containing the influence of each X-variable on the explanation of the variation in Y. Together the weight vectors will form a matrix, W, containing the variation in X that maximizes the covariance between the scores T and U for each calculated component. For PLS-DA this means that the weights, W, contain the variation in X that is correlated to the class separation described in Y. The Y-block matrix of weights is designated C. A matrix of X-loadings, P, is also calculated. These loadings are apart from interpretation used to perform the proper decomposition of )C

The PLS decomposition of X and Y can hence be described as follows:

X=TP′+E

Y=TC′+F

The PLS regression coefficients, B, are then given by:

B=W(P′W)⁻¹C′

The estimate of Y, Yₕₐₜ, can then be calculated according to the following formula:

Yₕₐₜ=XW(P′W)C′=XB

Both of the pattern recognition algorithms exemplified herein (PCA, PLS-DA) rely on extraction of linear associations between the input variables. When such linear relationships are insufficient, neural network-based pattern recognition techniques can in some cases improve the ability to classify individuals on the basis of the many inter-related input variables (see, e.g., Ala-Korpela et al., 1995; Hiltunen et al., 1995). Nevertheless, the methods applied herein are sufficiently powerful to allow classification of the individuals studied, and they provide an additional benefit over neural network methods in that they allow some information to be gained as to what aspects of the input dataset were particularly important in allowing classification to be made.

Spurious or irregular data in spectra (“outliers”), which are not representative, are preferably identified and removed. Common reasons for irregular data (“outliers”) include spectral artefacts such as poor phase correction, poor baseline correction, poor chemical shift referencing, poor water suppression, and biological effects such as bacterial contamination, shifts in the pH of the biofluid, toxin- or disease-induced biochemical response, and other conditions, e.g., pathological conditions, which have metabolic consequences, e.g., diabetes.

Outliers are identified in different ways depending on the method of analysis used. For example, when using principal component analysis (PCA), small numbers of samples lying far from the rest of the replicate group can be identified by eye as outliers. A more objective means of identification for PCA is to use the Hotelling's T Test which is the multivariate version of the well known Student's T test used in univariate statistics. For any given sample, the T2 value can be calculated and this is compared with a standard value within which a chosen fraction (e.g., 95%) of the samples would normally lie. Samples with T2 values substantially outside this limit can then be flagged as outliers.

Also, when using more sophisticated supervised methods, such as SIMCA or PNNs, a similar method is used. A confidence level (e.g., 95%) is selected and the region of multivariate space corresponding to confidence values above this limit is determined. This region can be displayed graphically in several different ways (for example by plotting the critical T2 ellipse on a PCA scores plot). Any samples falling outside the high confidence region are flagged as potential outliers.

Confidence Limits for outlier detection are also calculated in the residual direction expressed as the distance to model in X (DModX).

Briefly, DModX is the perpendicular distance of an object to the principal component (or to the plane or hyper plane made up by two or more principal components). In the SIMCA software, DModX is calculated as:

DModX=v*sqrt(e²/K−A)

wherein e is the residual for a single observation;

K is the number of original variables in the data set;

A is the number of principal components in the model;

v is a correction factor, based on the number of observations (N) and the number of principal components (A), and is slightly larger than one.

The outliers in this direction are not as severe as those occurring in the score direction but should always be carefully examined before making a decision whether to include them in the modelling or not. In general, all outliers are thoroughly investigated, for example, by examining the contributing loadings and distance to model (DModX) as well as visually inspecting the original NMR spectrum for deviating features, before removing them from the model. Outlier detection by automatic algorithm is a possibility using the features of scores and residual distance to model (DModX) described above.

When using PLS methods, the distance to the model in Y (DmodY) can also be calculated in the same way.

Data Filtering

Although pattern recognition methods may be applied to “unfiltered” data, it is often preferable to first filter data to removed irrelevant variation.

In one method, latent variables which are of no interest may be removed by “filtering.”

Examples of filtering methods include the regression of descriptor variables against an index based on sample class to eliminate variables with low correlation to the predefined classes. Related methods include target rotation (see, e.g., Kvalheim et al., 1989) and PCT filtering (see, e.g., Sun, 1997). In these methods, the removed variation is not necessarily completely uncorrelated with sample class (i.e., orthogonal).

In another method, latent variables which are orthogonal to some variation or class index of interest are removed by “orthogonal filtering.” Here, variation in the data which is not correlated to (i.e., is orthogonal to) the class separating variation of interest may be removed. Such methods are, in general, more efficient than non-orthogonal filtering methods.

Various orthogonal filtering methods have been described (see, e.g., Wold et al., 1998a; Fearn, 2000; Anderson, 1999; Westerhuis et al., 2001; Wise et al., 2001).

One preferred orthogonal filtering method is conventionally referred to as Orthogonal Signal Correction (OSC), wherein latent variables orthogonal to the variation of interest are removed. See, for example, Wold et al., 1998a.

The class identity is used as a response vector, Y, to describe the variation between the sample classes. The OSC method then locates the longest vector describing the variation between the samples which is not correlated with the Y-vector, and removes it from the data matrix. The resultant dataset has been filtered to allow pattern recognition focused on the variation correlated to features of interest within the sample population, rather than non-correlated, orthogonal variation.

OSC is a method for spectral filtering that solves the problem of unwanted systematic variation in the spectra by removing components, latent variables, orthogonal to the response calibrated against. In PLS, the weights, w, are calculated to maximise the covariance between X and Y. In OSC, in contrast, the weights, w, are calculated to minimize the covariance between X and Y, which is the same as calculating components as close to orthogonal to Y as possible. These components, orthogonal to Y, containing unwanted systematic variation are then subtracted from the spectral data, X, to produce a filtered predictor matrix describing the variation of interest. Briefly, OSC can be described as a bilinear decomposition of the spectral matrix, X, in a set of scores, T**, and a set of corresponding loadings, P**, containing varition orthogonal to the response, Y. The unexplained part or the residuals, E, is equal to the filtered X-matrix, X_{osc}, containing less unwanted variation. The decomposition is described by the following equation:

X=T** P**′+E

X_{osc}=E

The OSC procedure starts by calculation of the first latent variable or principal component describing the variation in the data, X. The calculation is done according to the NIPALS algorithm.

X=t p′+E

The first score vector, t, which is a summary of the between sample variation in X, is then orthogonalized against response (Y), giving the orthogonalized score vector t*.

t*=(I−Y(Y′Y)⁻¹Y′)t

After orthogonalization, the PLS weights, w, are calculated with the aim of making Xw=t*. By doing this, the weights, w, are set to minimize the covariance between X and Y. The weights, w, are given by:

w=x−t*

An estimate of the orthogonal score t** is calculated from:

t**=Xw

The estimate or updated score vector t** is then again orthogonalized to Y, and the iteration proceeds until t** has converged. This will ensure that t** will converge towards the longest vector orthogonal to response Y, still giving a good description of the variation in X. The data, X, can then be described as the score, t**, orthogonal to Y, times the corresponding loading vector p**, plus the unexplained part, the residual, E.

X=t** p**′+E

The residual, E, equals the filtered X, X_{osc}, after subtraction of the first component orthogonal to the response Y.

E=X−t** p**′

Xosc=E

If more than one component needs to be removed, the same procedure is repeated using the residual, E, as the starting data matrix, X.

New external data not present in the model calculation must be treated according to filtering of the modelling data. This is done by using the calculated weights, w, from the filtering to calculate a score vector, t_{new}, for the new data, X_{new}.

t_{new}=X_{new}W

By subtracting t_{new} times the loading vector from the calibration, p**, from the new external data, X_{new}, the residual, E_{new}, will be the resulting OSC filtered matrix for the new external data.

E_{new}=X_{new}−t_{new}P**

If PCA suggests separation between the classes under investigation, orthogonal signal correction (OSC) can be used to optimize the separation, thus improving the performance of subsequent multivariate pattern recognition analysis and enhancing the predictive power of the model. In the examples described herein, both PCA and PLS-DA analyses were improved by prior application of OSC.

An example of a typical OSC process includes the following steps:

(a) ¹H NMR data are segmented using AMIX, normalised, and optionally scaled and/or mean centered. The default for orthogonal filtering of spectral data is to use only mean centered data, which means that the mean for each variable (spectral bucket) is subtracted from each single variable in the data matrix.

(b) a response vector (y) describing the class separating variation is created by assigning class membership to each sample.

(c) one latent variable orthogonal to the response vector (y) is removed according to the OSC algorithm.

(d) if desired, the removed orthogonal variation can be viewed and interpreted in terms of scores (T) and loadings (P).

(e) the filtered data matrix, which contains less variation not correlated to class separation, is next used for further multivariate modelling after optional scaling and/or mean centering.

Any particular model is only as good as the data used to formulate it. Therefore, it is preferable that all modelling data and test data are obtained under the same (or similar) conditions and using the same (or similar) experimental parameters. Such conditions and parameters include, for example, sample type (e.g., plasma, serum), sample collection and handling protocol, sample dilution, NMR analysis (e.g., type, field strength/frequency, temperature), and data-processing (e.g., referencing, baseline correction, normalisation). If appropriate, it may be desirable to formulate models for a particular subgroup of cases, e.g., according to any of the parameters mentioned above (e.g., field strength/frequency), or others, such as sex, age, ethnicity, medical history, lifestyle (e.g., smoker, nonsmoker), hormonal status (e.g., pre-menopausal, post-menopausal).

In general, the quality of the model improves as the amount of modelling data increases. Nonetheless, as shown in the examples below, even relatively small sets of modelling data (e.g., about 50-100 subjects) is sufficient to achieve a confident classification (e.g., diagnosis).

A typical unsupervised modelling process includes the following steps:

(a) optionally scaling and/or mean centering modelling data;

(b) classifying data (e.g., as control or positive, e.g., diseased);

(c) fitting the model (e.g., using PCA, PLS-DA);

(d) identifying and removing outliers, if any;

(e) re-fitting the model;

(f) optionally repeating (c), (d), and (e) as necessary.

Optionally (and preferably), data filtering is performed following step (d) and before step (e). Optionally (and preferably), orthogonal filtering (e.g., OSC) is performed following step (d) and before step (e).

An example of a typical PLS-DA modelling process, using OSC filtered data, includes the following steps:

(a) OSC filtered data is optionally scaled and/or mean centered.

(b) a response vector (y) describing the class separating variation is created by assigning class membership to all samples.

(c) a PLS regression model is calculated between the OSC filtered data and the response vector (y). The calculated latent variables or PLS components will be focused on describing maximum separation between the known classes.

(d) the model is interpreted by viewing scores (T), loadings (P), PLS weights (V”), PLS coefficients (B) and residuals (E). Together they will function as a means for describing the separation between the classes as well as provide an explanation to the observed separation.

Once the model has been calculated, it may be verified using data for samples of known class which were not used to calculate the model. In this way, the ability of the model to accurately predict classes may be tested. This may be achieved, for example, in the method above, with the following additional step:

(e) a set of external samples, with known class belonging, which were not used in the (e.g., PLS) model calculation is used for validation of the model's predictive ability. The prediction results are investigated, fore example, in terms of predicted response (y_{pred}), predicted scores (T_{pred}), and predicted residuals described as predicted distance to model (DmodX_{pred}).

The model may then be used to classify test data, of unknown class. Before classification, the test data are numerically pre-processed in the same manner as the modelling data.

Interpreting the output from the pattern recognition (PR) analysis provides useful information on the biomarkers responsible for the separation of the biological classes. Of course, the PR output differs somewhat depending on the data analysis method used. As mentioned above, methods for PR and interpretation of the results are known in the art. Interpretation methods for two PR techniques (PCA and PLS-DA) are discussed briefly herein.

Interpreting PCA Results

The data matrix (X) is built up by N observations (samples, rats, patients, etc.) and K variables (spectral buckets carrying the biomarker information in terms of ¹H-NMR resonances).

In PCA, the N*K matrix (X) is decomposed into a few latent variables or principal components (PCs) describing the systematic variation in the data. Since PCA is a bilinear decomposition method, each PC can be divided into two vectors, scores (t) and loadings (p). The scores can be described as the projection of each observation on to each PC and the loadings as the contribution of each variable (spectral bucket) to the PC expressed in terms of direction.

Any clustering of observations (samples) along a direction found in scores plots (e.g., PC1 versus PC2) can be explained by identifying which variables (spectral buckets) have high loadings for this particular direction in the scores. A high loading is defined as a variable (spectral bucket) that changes between the observations in a systematic way showing a trend which matches the sample positions in the scores plot. Each spectral bucket with a high loading, or a combination thereof, is defined by its ¹H NMR chemical shift position; this is its diagnostic spectral window. These chemical shift values then allow the skilled NMR spectroscopist to examine the original NMR spectra and identify the molecules giving rise to the peaks in the relevant buckets; these are the biomarkers. This is typically done using a combination of standard 1- and 2-dimensional NMR methods.

If, in a scores plot, separation of two classes of sample can be seen in a particular direction, then examination of those loadings which are in the same direction as in the scores plots indicates which loadings are important for the class identification. The loadings plot shows points which are labelled according to the bucket chemical shift. This is the ¹H NMR spectroscopic chemical shift which corresponds to the centre of the bucket. This bucket defines a diagnostic spectral window. Given a list of these bucket identifiers, the skilled NMR spectroscopist then re-examines the ¹H NMR spectra and identifies, within the bucket width, which of several possible NMR resonances are changed between the two classes. The important resonance is characterised in terms of exact chemical shift, intensity, and peak multiplicity. Using other NMR experiments, such as 2-D NMR spectroscopy and/or separation of the specific molecule using HPLC-NMR-MS for example, other resonances from the same molecule are identified and ultimately, on the basis of all of the NMR data and other data if appropriate, an identification of the molecule (biomarker) is made.

In a classification situation as described herein, one procedure for finding relevant biomarkers using PCA is as follows:

(a) PCA of the data matrix (X) containing N observations belonging to either of two known classes (healthy or diseased). The description of the observations lies in the K variables (spectral buckets) containing the biomarker information in terms of ¹H NMR resonances.

(b) Interpretation of the scores (t) to find the direction for the separation between the two known classes in X.

(c) Interpretation of loadings (p) reveals which variables (spectral buckets) have the largest impact on the direction for separation described in the scores (t). This identifies the relevant diagnostic spectral windows.

(d) Assignment of the spectral buckets or combinations thereof to certain biomarkers. This is done, for example, by interpretation of the resonances in ¹H NMR spectra and by using previously assigned spectra of the same type as a library for assignments.

Interpreting PLS-DA Results

In PLS-DA, which is a regression extension of the PCA method, the options for interpretation are more extensive compared to the PCA case. PLS-DA performs a regression between the data matrix (X) and a “dummy matrix” (Y) containing the class membership information (e.g., samples may be assigned the value 1 for healthy and 2 for diseased classes). The calculated PLS components will describe the maximum covariance between X and Y which in this case is the same as maximum separation between the known classes in X. The interpretation of scores (t) and loadings (p) is the same in PLS-DA as in PCA. Interpretation of the PLS weights (w) for each component provides an explanation of the variables in X correlated to the variation in Y. This will give biomarker information for the separation between the classes.

Since PLS-DA is a regression method, the features of regression coefficients (b) can also be used for discovery and interpretation of biomarkers. The regression coefficients (b) in PLS-DA provide a summary of which variables in X (spectral buckets) that are most important in terms of both describing variation in X and correlating to Y. This means that variables (spectral buckets) with high regression coefficients are important for separating the known classes in X since the Y matrix against which it is correlated only contains information on the class identity of each sample.

Again, as discussed above, the scores plot is examined to identify important loadings, diagnostic spectral windows, relevant NMR resonances, and ultimately the associated biomarkers.

In a classification situation as described herein, one procedure for finding relevant biomarkers using PLS-DA is as follows:

(a) A PLS model between the N*K data matrix (X) against a “dummy matrix” Y, containing information on class membership for the observations in X, is calculated yielding a few latent variables (PLS components) describing maximum separation between the two classes in X (e.g., healthy and diseased).

(b) Interpretation of the scores (t) to find the direction for the separation between the two known classes in X.

(c) Interpretation of loadings (p) revealing which variables (spectral buckets) have the largest impact on the direction for separation described in the scores (t); these are diagnostic spectral windows.

In PLS-DA, a variable importance plot (VIP) is another method of evaluating the significance of loadings in causing a separation of class of sample in a scores plot. Typically, the VIP is a squared function of PLS weights, and therefore only positive numerical values are encountered; in addition, for a given model, there is only one set of VIP-values. Variables with a VIP value of greater than 1 are considered most influential for the model. The VIP shows each loading in a decreasing order of importance for class separation based on the PLS regression against class variable.

A (w*c) plot is another diagnostic plot obtained from a PLS-DA analysis. It shows which descriptors are mainly responsible for class separation. The (w*c) parameters are an attempt to describe the total variable correlations in the model, i.e., between the descriptors (e.g., NMR intensities in buckets), between the NMR descriptors and the class variables, and between class variables if they exist (in the present two class case, where samples are assigned by definition to class 1 and class 2 there is no correlation).

Thus for a situation in a scores plot (e.g., t1 vs. t2), if class I samples are clustered in the upper right hand quadrant and class 2 samples are clustered in the lower left hand quadrant, then the (w*c) plot will show descriptors also in these quadrants. Descriptors in the upper right hand quadrant are increased in class 1 compared to class 2 and vice versa for the lower left hand quadrant.

(d) Interpretation of PLS weights (w) reveals which variables (spectral buckets) in X are important for correlation to Y (class separation); these, too, are diagnostic spectral windows.

(e) Interpretation of the PLS regression coefficients (b) reveals an overall summary of which variables (spectral buckets) have the largest impact on the direction for separation described in the scores; these, too, are diagnostic spectral windows.

In a typical regression coefficient plot for ¹H NMR, each bar represents a spectral region (e.g., 0.04 ppm) and shows how the ¹H NMR profile of one class of samples differs from the ¹H NMR profile of a second class of samples. A positive value on the x-axis indicates there is a relatively greater concentration of metabolite (assigned using NMR chemical shift assignment tables) in one class as compared to the other class, and a negative value on the x-axis indicates a relatively lower concentration in one class as compared to the other class.

(f) Assignment of the spectral buckets or combinations thereof to certain biomarkers. This is done, for example, by interpretation of the resonances in ¹H NMR spectra and by using previously assigned spectra of the same type as a library for assignments.

Timed Sampling

The analysis methods described herein can be applied to a single sample, or alternatively, to a timed series of samples. These samples may be taken relatively close together in time (e.g., daily) or less frequently (e.g., monthly or yearly).

The timed series of samples may be used for one or more purposes, e.g., to make sequential diagnoses, applying the same classification method as if each sample were a single sample. This will allow greater confidence in the diagnosis compared to obtaining a single sample for the patient, or alternatively to monitor temporal changes in the subject (e.g., changes in the underlying condition being diagnosed, treated, etc.).

Alternatively, the timed series of samples can be collectively treated as a single dataset increasing the information density of the input dataset and hence increasing the power of the analysis method to identify weaker patterns.

As yet another alternative, the timed series of samples can be collectively processed to yield a single dataset in which the temporal changes (e.g., in each bin) is included as an extra list of variables (e.g., as in composite data sets). Temporal changes in the amount of (e.g., endogenous) diagnostic species may greatly improve the ability of the analysis method to accurate classify patterns (especially when patterns are weak).

Batch Modelling

The methods described herein, including their applications (e.g., diagnosis, prognosis), may be further improved by employing batch modelling.

Statistical batch processing can be divided into two levels of multivariate modelling. The lower or the observation level is usually based on Partial Least Squares (PLS) regression against time (or any other index describing process maturity), whereas the upper or batch level consists of a PCA based on the scores from the lower level PLS model. PLS can also be used in the upper level to correlate the matrix based on the lower level scores with the end properties of the separate batches. This is common in industrial applications where properties of the end product are used as a description of quality.

At the lower level of the Batch modelling the evolution of the studied process with time (maturity) can be monitored and interpreted in terms of PLS scores and loadings. Since the PLS performs a regression against sampling time (maturity), the calculated components will be focused on the evolution with time. The fact that the calculated PLS components are orthogonal to each other means that it is possible to detect independent time (maturity) profiles and also to interpret which measured variables are causing these profiles. Confidence limits are used for detection of deviating behaviour of any spectra at any time point for some optional significance level, usually 95% and/or 99%.

The residuals expressed as distance to model (DModX) is, at the lower level, another important tool for detecting outlying batches or deviating behaviour for a specific batch at a specific time point. The upper level or batch level provides the possibility to just look at the difference between the separate batches. This is done by using the lower level scores including all time points for each batch as new variables describing each single batch and then performing a PCA on this new data matrix. The features of scores, loadings and DmodX are used in the same way as for ordinary PCA analysis, with the exception that the upper level loadings can be traced back down to the lower level for a more detailed explanation in the original loadings.

Predictions for “new” batches can be done on both levels of the batch model. On the lower level monitoring of evolution with time using scores and DmodX is a powerful tool for detecting deviating behaviour from normality for batch at any time point. On the upper level prediction of single batch behaviour can be done in terms of scores and DmodX.

The definition of a batch process, and also a requirement for batch modelling, is a process where all batches have equal duration and are synchronised according to sample collection. For example, samples taken from a cohort of animals at identical fixed time points to monitor the effects of an administered xenobiotic substance.

The advantage of using batch modelling for such studies is the possibility of detecting known, or discovering new, metabolic processes which evolve with time in the lower level scores, and also the identification of the actual metabolites involved in the different processes from the contributing lower level loadings. The lower level analysis also makes it possible to differentiate between single observations (e.g., individual animals at specific time points).

Applications for the lower level modelling include, for example, distinguishing between undosed controls and dosed animals in terms of metabolic effects of dosing in certain time points; and creating models for normality and using the models as a classification tool for new samples, e.g., as normal or abnormal. This may be achieved using a PLS prediction of the new sample's class using the model describing normality. Decisions can then be made on basis of the combination of the predicted scores and residuals (DmodX).

An automated expert system can be used for early fault detection in the lower level batch modelling, and this can be used to further enhance the analysis procedure and improve efficiency.

The upper level provides the possibility of making predictions of new animals using the existing model. Abnormal animals can then be detected by judging predicted scores and residuals (DmodX) together. Since the upper level model is based on the lower level scores, the interpretation of an animal predicted to be abnormal can be traced back to the original lower level scores and loadings as well as the original raw variables making up the NMR spectra. Combining the upper and lower level for prediction of the status of a new animal, the classification can be based on four parameters: upper level scores and residuals (DmodX) and lover level scores and residuals (DModX). This demonstrates that batch modelling is an efficient tool for determining if an animal is normal or abnormal, and if the latter, why and when they are deviating from normality.

See, for example, Wold et al, 1998b and Eriksson et al., 1999.

Integrated Metabonomics

As discussed above, many of the methods of the present invention may also be applied to composite data or composite data sets. The term “composite data set,” as used herein, pertains to a spectrum (or data vector) which comprises spectral data (e.g., NMR spectral data, e.g., an NMR spectrum) as well as at least one other datum or data vector. Examples of other data vectors include, e.g., one or more other NMR spectral data, e.g., NMR spectra, e.g., obtained for the same sample using a different NMR technique; other types of spectra, e.g., mass spectra, numerical representations of images, etc.; obtained for the another sample, of the same sample type (e.g., blood, urine, tissue, tissue extract), but obtained from the subject at a different timepoint; obtained for another sample of different sample type (e.g., blood, urine, tissue, tissue extract) for the same subject; and the like.

Examples of other data including, e.g., one or more clinical parameters. Clinical parameters which are suitable for use in composite methods include, but are not limited to, the following:

(a) established clinical parameters routinely measured in hospital clincal labs: age; sex; body mass index; height; weight; family history; medication history; cigarette smoking; alcohol intake; blood pressure; full blood cell count (FBCs); red blood cells; white blood cells; monocytes; lymphocytes; neutrophils; eosinophils; basophils; platelets; haematocrit; haemoglobin; mean corpuscular volume and related haemodilution indicators; fibrinogen; functional clotting parameters (thromoboplastin and partial thromboplastin); electrolytes (sodium, potassium, calcium, phosphate); urea; creatinine; total protein; albumin; globulin; bilirubin; protein markers of liver function (alanine aminotransferase, alkaline phosphatase, gamma glutamyl transferase); glucose; Hba1c (a measure of glucose-Haemoglobin conjugates used to monitor diabetes); lipoprotein profile; total cholesterol; LDL; HDL; triglycerides; blood group.

(b) established research parameters routinely measured in research laboratories but not usually measured in hospitals: hormonal status; testosterone; estrogen; progesterone; follicle stimulating hormone; inhibin; transforming growth factor-beta1; Transforming growth factor-beta2; chemokines; MCP-1; eotaxin; plasminogen activator inhibitor-1; cystatin C.

(c) early-stage research parameters measured in one or a small number of specialist labs: antibodies to sRII; antibodies to blood group A antigen; antibodies to blood group B antigen; immunoglobulin (IgD) against alpha-gal; immunoglobulin (IgD) against penta-gal.

Diagnostic Spectral Windows

As discussed above, many of the methods of the present invention involve relating NMR spectral intensity at one or more predetermined diagnostic spectral windows with a predetermined condition.

Examples of methods for identifying one or more suitable diagnostic spectral windows for a given condition, using, for example, pattern recognition methods, are described herein.

The term “diagnostic spectral window,” as used herein, pertains to narrow range of chemical shift (Δδ) values encompassing an index value, δᵣ (that is, δᵣ falls within the range Δδ). Each index value, and its associated spectral window, define a range of chemical shift (Δδ) in which the NMR spectral intensity is indicative of the presence of one or more chemical species.

For 2D NMR methods, the diagnostic spectral window refers to a chemical shift patch (Δδ₁, Δδ₂) which encompasses an index value, [δᵣ₁, δᵣ₂]. For 3D NMR methods, the diagnostic spectral window refers to a chemical shift volume (Δδ₁, Δδ₂, Δδ₃) which encompasses an index value, [δᵣ₁, δᵣ₂, δᵣ₃].

In one embodiment, the spectral window is centred with respect to its index value (e.g., δᵣ=1.30;|Δδ|=δ0.04, and Δδ1.28-1.32).

The breadth of the range, |Δδ|, is determined largely by the spectroscopic parameters, such as field strength/frequency, temperature, sample viscosity, etc. The breadth of the range is often chosen to encompass a typical spin-coupled multiplet pattern. For peaks whose position varies with sample pH, the breadth of the range is may be widened to encompass the expected range of positions.

Typically, the breadth of the range, |Δδ|, is from about δ 0.001 to about δ 0.2.

In one embodiment, the breadth is from about δ 0.005 to about δ 0.1.

In one embodiment, the breadth is from about δ 0.005 to about δ 0.08.

In one embodiment, the breadth is from about δ 0.01 to about δ 0.08.

In one embodiment, the breadth is from about δ 0.02 to about δ 0.08.

In one embodiment, the breadth is from about δ 0.005 to about δ 0.06.

In one embodiment, the breadth is from about δ 0.01 to about δ 0.06.

In one embodiment, the breadth is from about δ 0.02 to about δ 0.06.

In one embodiment, the breadth is about δ 0.04.

In one embodiment, the breadth is equal to the “bucket” or“bin” width. In one embodiment, the breadth is equal to an integer multiple of the “bucket” or “bin” width.

Although the diagnostic spectral windows are determined in relation to the condition under study, the precise index values for such windows may vary in accordance with the experimental parameters employed, for example, the digital resolution in the original spectra, the width of the buckets used, the temperature of the spectral data acquisition, etc. The exact composition of the sample (e.g., biofluid, tissue, etc.) can affect peak positions by compartmentation, metal complexation, protein-small molecule binding, etc.

The observation frequency will have an effect because of different degrees of peak overlap and of first/second order nature of spectra.

In one embodiment, said one or more predetermined diagnostic spectral windows is: a single predetermined diagnostic spectral window.

In one embodiment, said one or more predetermined diagnostic spectral windows is: a plurality of predetermined diagnostic spectral windows. In practice, this may be preferred.

Although the theoretical limit on the number of predetermined diagnostic spectral windows is a function of the data density (e.g., the number of variables, e.g., buckets), typically the number of predetermined diagnostic spectral windows is from I to about 30. It is possible for the actual number to be in any sub-range within these general limits. Examples of lower limits include 1, 2, 3, 4, 5, 6, 8, 10, and 15. Examples of upper limits include 3, 4, 5, 6, 8, 10, 15, 20, 25, and 30.

In one embodiment, the number is from 1 to about 20.

In one embodiment, the number is from 1 to about 15.

In one embodiment, the number is from 1 to about 10.

In one embodiment, the number is from 1 to about 8.

In one embodiment, the number is from 1 to about 6.

In one embodiment, the number is from 1 to about 5.

In one embodiment, the number is from 1 to about 4.

In one embodiment, the number is from 1 to about 3.

In one embodiment, the number is 1 or 2.

In one embodiment, said one or more predetermined diagnostic spectral windows is: a plurality of diagnostic spectral windows; and, said NMR spectral intensity at one or more predetermined diagnostic spectral windows is: a combination of a plurality of NMR spectral intensities, each of which is NMR spectral intensity for one of said plurality of predetermined diagnostic spectral windows.

In one embodiment, said combination is a linear combination.

In one embodiment, at least one of said one or more predetermined diagnostic spectral windows encompasses a chemical shift value for an NMR resonance of a diagnostic species (e.g., a ¹H NMR resonance of a diagnostic species).

In one embodiment, each of a plurality of said one or more predetermined diagnostic spectral windows encompasses a chemical shift value for an NMR resonance of a diagnostic species (e.g., a ¹H NMR resonance of a diagnostic species).

In one embodiment, each of said one or more predetermined diagnostic spectral windows encompasses a chemical shift value for an NMR resonance of a diagnostic species (e.g., a ¹H NMR resonance of a diagnostic species).

Diagnostic Spectral Windows—Osteoarthritis

It is believed that the index values, and the associated diagnostic spectral windows, primarily reflect the species described in Table 1-OA and/or Table 2-OA.

In one embodiment, said predetermined diagnostic spectral windows are defined by one or more index values, δᵣ, corresponding to the bucket regions listed in Table 1-OA and/or Table 2-OA.

In one embodiment, said predetermined diagnostic spectral windows are defined by one or more index values, δᵣ, corresponding to the bucket regions listed in Table 1-OA and/or Table 2-OA, and breadth of the range value, |Δδ| about 0.04.

In one embodiment, said predetermined diagnostic spectral windows are defined by one or more index values, δᵣ, corresponding to the bucket regions listed in Table 1-OA and/or Table 2-OA, and which are determined using the conditions set forth in the section entitled “NMR Experimental Parameters.”

Diagnostic Species and Biomarkers

The index values, and the associated diagnostic spectral windows, define ranges of chemical shift in which NMR spectral intensity is indicative of the presence of one or more chemical species, one or more of which are diagnostic species (e.g., biomarkers), for example, for a condition (e.g., indication) under study.

In one embodiment, said one or more diagnostic species are endogenous diagnostic species.

In one embodiment, said one or more diagnostic species are associated with NMR spectral intensity at predetermined diagnostic spectral windows.

In one embodiment, said one or more diagnostic species are a plurality of diagnostic species (i.e., a combination of diagnostic species).

In one embodiment, said one or more diagnostic species is a single diagnostic species.

The term “endogenous species,” as used herein, pertains to chemical species which originated from the subject under study, for example, which were present in the sample of the subject.

Once an index value, and its associated diagnostic spectral window, is identified (e.g., by the application of modelling methods as described herein), it is often possible to identify one or more putative biomarkers which give rise to NMR spectral intensity in that particular window.

The (e.g., integrated) NMR spectral intensity in a particular spectral window (e.g., bucket) is the sum of the spectral intensity for all of the NMR peaks in that window. Usually for small molecules which give sharp NMR peaks, it is possible to examine the raw NMR data and determine which of the peaks is responsible for that particular spectral window being selected as significant by the applied pattern recognition method. The relevant peak(s) are then assigned.

Such assignments may be made, for example, by reference to published data; by comparison with spectra of authentic materials; by standard addition of an authentic reference standard to the sample; by separating the individual component, e.g., by using HPLC-NMR and identifying it using NMR and mass spectrometry. Additional confirmation of assignments is usually sought from the application of other NMR methods, including, for example, 2-dimensional (2D) NMR methods.

In another approach, concentrations of candidate chemical species are measured by another specific method (e.g., ELISA, chromatography, RIA, etc.) and compared with the spectral intensity observed in the relevant diagnostic spectral window, and any correlation noted. This will reveal how much of the variance in the diagnostic spectral window is contributed by the candidate chemical species. This may also reveal that suspected diagnostic species are, in fact, not highly correlated with the condition under examination.

Methods of Identifying Diagnostic Species

Thus, the methods described herein also facilitate the identification of species (often referred to as biomarkers or diagnostic species) which are indicative (e.g., diagnostic) of a particular condition. For example, particular metabolites (e.g., in blood, urine, etc.) may be diagnostic of a particular condition.

One aspect of the present invention pertains to a method of identifying such diagnostic species (e.g., biomarkers), as described herein.

One aspect of the present invention pertains to a method of identifying a diagnostic species, or a combination of a plurality of diagnostic species, for a predetermined condition, said method comprising the steps of:

(a) applying a multivariate statistical analysis method to experimental data;

wherein said experimental data comprises at least one data comprising experimental parameters measured for each of a plurality of experimental samples;

wherein said experimental samples define a class group consisting of a plurality of classes;

wherein at least one of said plurality of classes is a class associated with said predetermined condition, e.g., a class associated with the presence of said predetermined condition;

wherein at least one of said plurality of classes is a class not associated with said predetermined condition, e.g., a class associated with the absence of said predetermined condition;

wherein each of said experimental samples is of known class selected from said class group;

and:

(b) identifying one or more critical experimental parameters;

wherein each of said critical experimental parameters is statistically significantly different for classes of said class group, e.g., is statistically significant for discriminating between classes of said class group; and,

(c) matching each of one or more of said one or more critical experimental parameters with said diagnostic species;

or

(b) identifying a combination of a plurality of critical experimental parameters;

wherein said combination of a plurality of critical experimental parameters is statistically significantly different for classes of said class group, e.g., is statistically significant for discriminating between classes of said class group; and,

(c) matching each of one or more of said plurality of critical experimental parameters with said combination of a plurality of diagnostic species.

In one embodiment, one or more of said critical experimental parameters is a spectral parameter (i.e., a critical experimental spectral parameter); and said identifying and matching steps are:

(b) identifying one or more critical experimental spectral parameters; and,

(c) matching each of one or more of said one or more critical experimental spectral parameters with a spectral feature, e.g., a spectral peak; and

matching one or more of said spectral peaks with said diagnostic species;

or.

(b) identifying a combination of a plurality of critical experimental spectral parameters; and,

(c) matching each of a plurality of said plurality of critical experimental spectral parameters with a spectral feature, e.g., a spectral peak; and

matching one or more of said spectral peaks with said combination of a plurality of diagnostic species.

In one embodiment, said multivariate statistical analysis method is a multivariate statistical analysis method which employs a pattern recognition method.

In one embodiment, said multivariate statistical analysis method is, or employs PCA.

In one embodiment, said multivariate statistical analysis method is, or employs PLS.

In one embodiment, said multivariate statistical analysis method is, or employs PLS-DA.

In one embodiment, said multivariate statistical analysis method includes a step of data filtering.

In one embodiment, said multivariate statistical analysis method includes a step of orthogonal data filtering.

In one embodiment, said multivariate statistical analysis method includes a step of OSC.

In one embodiment, said experimental parameters comprise spectral data.

In one embodiment, said experimental parameters comprise both spectral data and non-spectral data (and is referred to as a “composite experimental data”).

In one embodiment, said experimental parameters comprise NMR spectral data.

In one embodiment, said experimental parameters comprise both NMR spectral data and non-NMR spectral data.

In one embodiment, said NMR spectral data comprises ¹H NMR spectral data and/or ¹³C NMR spectral data.

In one embodiment, said NMR spectral data comprises ¹H NMR spectral data.

In one embodiment, said non-spectral data is non-spectral clinical data.

In one embodiment, said non-NMR spectral data is non-spectral clinical data.

In one embodiment, said critical experimental parameters are spectral parameters.

In one embodiment, said class group comprises classes associated with said predetermined condition (e.g., presence, absence, degree, etc.).

In one embodiment, said class group comprises exactly two classes.

In one embodiment, said class group comprises exactly two classes: presence of said predetermined condition; and absence of said predetermined condition.

In one embodiment, said class associated with said predetermined condition is a class associated with the presence of said predetermined condition.

In one embodiment, said class not associated with said predetermined condition is a class associated with the absence of said predetermined condition.

In one embodiment, said method further comprises the additional step of:

(d) confirming the identity of said diagnostic species.

One aspect of the present invention pertain to novel diagnostic species (e.g., biomarker) which are identified by such a method.

One aspect of the present invention pertains to one or more diagnostic species (e.g., biomarkers) which are identified by such a method for use in a method of classification (e.g., diagnosis).

One aspect of the present invention pertains to a method of classification (e.g., diagnosis) which employs or relies upon one or more diagnostic species (e.g., biomarkers) which are identified by such a method.

One aspect of the present invention pertains to use of one or more diagnostic species (e.g., biomarkers) which are identified by such a method in a method of classification (e.g., diagnosis).

One aspect of the present invention pertains to an assay for use in a method of classification (e.g., diagnosis), which assay relies upon one or more diagnostic species (e.g., biomarkers) which are identified by such a method.

One aspect of the present invention pertains to use of an assay in a method of classification (e.g., diagnosis), which assay relies upon one or more diagnostic species (e.g., biomarkers) which are identified by such a method.

Diagnostic Species—Osteoarthritis

In one embodiment, at least one of said one or more predetermined diagnostic species is a species described in Table 1-OA and/or Table 2-OA.

In one embodiment, each of a plurality of said one or more predetermined diagnostic species is a species described in Table 1-OA and/or Table 2-OA.

In one embodiment, each of said one or more predetermined diagnostic species is a species described in Table 1-OA and/or Table 2-OA.

Amount or Relative Amount

As discussed above, many of the methods of the present invention involve classification on the basis of an amount, or a relative amount, of one or more diagnostic species.

In one embodiment, said classification is performed on the basis of an amount, or a relative amount, of a single diagnostic species.

In one embodiment, said classification is performed on the basis of an amount, or a relative amount, of a plurality of diagnostic species.

In one embodiment, said classification is performed on the basis of an amount, or a relative amount, of each of a plurality of diagnostic species.

In one embodiment, said classification is performed on the basis of a total amount, or a relative total amount, of a plurality of diagnostic species.

In one embodiment (wherein said one or more diagnostic species is: a plurality of diagnostic species), said amount of, or relative amount of one or more diagnostic species is: a combination of a plurality of amounts, or relative amounts, each of which is the amount of, or relative amount of one of said plurality of diagnostic species.

In one embodiment, said combination is a linear combination.

The term “amount,” as used in this context, pertains to the amount regardless of the terms of expression.

The term “amount,” as used herein in the context of “amount of, or relative amount of (e.g., diagnostic) species,” pertains to the amount regardless of the terms of expression.

Absolute amounts may be expressed, for example, in terms of mass (e.g., μg), moles (e.g., μmol), volume (i.e., μL), concentration (molarity, μg/mL, pg/g, wt %, vol %, etc.), etc.

Relative amounts may be expressed, for example, as ratios of absolute amounts (e.g., as a fraction, as a multiple, as a %) with respect to another chemical species. For example, the amount may expressed as a relative amount, relative to an internal standard, for example, another chemical species which is endogenous or added.

The amount may be indicated indirectly, in terms of another quantity (possibly a precursor quantity) which is indicative of the amount. For example, the other quantity may be a spectrometric or spectroscopic quantity (e.g., signal, intensity, absorbance, transmittance, extinction coefficient, conductivity, etc.; optionally processed, e.g., integrated) which itself indicative of the amount.

The amount may be indicated, directly or indirectly, in regard to a different chemical species (e.g., a metabolic precursor, a metabolic product, etc.), which is indicative the amount.

Diagnostic Shift

As discussed above, many of the methods of the present invention involve classification on the basis of a modulation, e.g., of NMR spectral intensity at one or more predetermined diagnostic spectral windows; of the amount, or a relative amount, of diagnostic species; etc. In this context, “modulation” pertains to a change, and may be, for example, an increase or a decrease. In one embodiment, said “a modulation of” is “an increase or decrease in.”

In one embodiment, the modulation (e.g., increase, decrease) is at least 10%, as compared to a suitable control. In one embodiment, the modulation (e.g., increase, decrease) is at least 20%, as compared to a suitable control. In one embodiment, the modulation is a decrease of at least 50% (i.e., a factor of 0.5). In one embodiment, the modulation is a increase of at least 100% (i.e., a factor of 2).

Each of a plurality of predetermined diagnostic spectral windows, and each of a plurality of diagnostic species, may have independent modulations, which may be the same or different. For example, if there are two predetermined diagnostic spectral windows, NMR spectral intensity may increase in one window and decrease in the other window. In this way, combinations of modulations of NMR spectral intensity in different diagnostic spectral windows may be diagnostic. Similarly, if there are two diagnostic species, the amount of one may increase, and the amount of the other may decrease. Again, combinations of modulations of amounts, or relative amounts of, different diagnostic species may be diagnostic. See, for example, the data in the Examples below, which illustrate cases where different species have different modulations.

The term “diagnostic shift,” as used herein, pertains a modulation (e.g., increase, decrease), as compared to a suitable control.

A diagnostic shift may be in regard to, for example, NMR spectral intensity at one or more predetermined diagnostic spectral windows; or the amount of, or relative amount of, diagnostic species.

Control Samples, Control Subjects, Control Data

Suitable controls are usually selected on the basis of the organism (e.g., subject, patient) under study (test subject, study subject, etc.), and the nature of the study (e.g., type of sample, type of spectra, etc.). Usually, controls are selected to represent the state of “normality.” As described herein, deviations from normality (e.g., higher than normal, lower than normal) in test data, test samples, test subjects, etc. are used in classification, diagnosis, etc.

For example, in most cases, control subjects are the same species as the test subject and are chosen to be representative of the equivalent normal (e.g., healthy) organism. A control population is a population of control subjects. If appropriate, control subjects may have characteristics in common (e.g., sex, ethnicity, age group, etc.) with the test subject. If appropriate, control subjects may have characteristics (e.g., age group, etc.) which differ from those of the test subject. For example, it may be desirable to choose healthy 20-year olds of the same sex and ethnicity as the study subject as control subjects.

In most cases, control samples are taken from control subjects. Usually, control samples are of the same sample type (e.g., serum), and are collected and handled (e.g., treated, processed, stored) under the same or similar conditions, as the sample under study (e.g., test sample, study sample).

In most cases, control data (e.g., control values) are obtained from control samples which are taken from control subjects. Usually, control data (e.g., control data sets, control spectral data, control spectra, etc.) are of the same type (e.g., 1-D ¹H NMR, etc.), and are collected and handled (e.g., recorded, processed) under the same or similar conditions (e.g., parameters), as the test data.

Implementation

The methods of the present invention, or parts thereof, may be conveniently performed electronically, for example, using a suitably programmed computer system.

One aspect of the present invention pertains to a computer system or device, such as a computer or linked computers, operatively configured to implement a method of the present invention, as described herein.

One aspect of the present invention pertains to computer code suitable for implementing a method of the present invention, as described herein, on a suitable computer system.

One aspect of the present invention pertains to a computer program comprising computer program means adapted to perform a method according to the present invention, as described herein, when said program is run on a computer.

One aspect of the present invention pertains to a computer program, as described above, embodied on a computer readable medium.

One aspect of the present invention pertains to a data carrier which carries computer code suitable for implementing a method of the present invention, as described herein, on a suitable computer.

In one embodiment, the above-mentioned computer code or computer program includes, or is accompanied by, computer code and/or computer readable data representing a predictive mathematical model, as described herein.

In one embodiment, the above-mentioned computer code or computer program includes, or is accompanied by, computer code and/or computer readable data representing data from which a predictive mathematical model, as described herein, may be calculated.

One aspect of the present invention pertains to computer code and/or computer readable data representing a predictive mathematical model, as described herein.

One aspect of the present invention pertains to a data carrier which carries computer code and/or computer readable data representing a predictive mathematical model, as described herein.

One aspect of the present invention pertains to a computer system or device, such as a computer or linked computers, programmed or loaded with computer code and/or computer readable data representing a predictive mathematical model, as described herein.

Computers may be linked, for example, internally (e.g., on the same circuit board, on different circuit boards which are part of the same unit), by cabling (e.g., networking, ethernet, internet), using wireless technology (e.g., radio, microwave, satellite link, cell-phone), etc., or by a combination thereof.

Examples of data carriers and computer readable media include chip media (e.g., ROM, RAM, flash memory (e.g., Memory Stick™, Compact Flash™, Smartmedia™), magnetic disk media (e.g., floppy disks, hard drives), optical disk media (e.g., compact disks (CDs), digital versatile disks (DVDs), magneto-optical (MO) disks), and magnetic tape media.

Although the ¹H-NMR spectra analysed here were generated using a conventional (and hence large and expensive) 600 MHz NMR spectrometer, on-going technological advances suggest that spectrometers of similar resolving power may soon be available as desktop units (provided the sample to be analyzed is small, as is the case with plasma or serum samples). Such units, together with a personal computer to perform automated pattern recognition, may soon be available not only in large hospitals but also in the primary healthcare milieu.

One aspect of the present invention pertains to a system (e.g., an “integrated analyser”, “diagnostic apparatus”) which comprises:

(a) a first component comprising a device for obtaining NMR spectral intensity data for a sample (e.g., a NMR spectrometer, e.g., a Bruker INCA 500 MHz); and,

(b) a second component comprising computer system or device, such as a computer or linked computers, operatively configured to implement a method of the present invention, as described herein, and operatively linked to said first component.

In one embodiment, the first and second components are in close proximity, e.g., so as to form a single console, unit, system, etc. In one embodiment, the first and second components are remote (e.g., in separate rooms, in separate buildings).

A simple process for the use of such a system is described below.

In a first step, a sample (e.g., blood, urine, etc.) is obtained from a subject, for example, by a suitably qualified medical technician, nurse, etc., and the sample is processed as required. For example, a blood sample may be drawn, and subsequently processed to yield a serum sample, within about three hours.

In a second step, the sample is appropriately processed (e.g., by dilution, as described herein), and an NMR spectrum is obtained for the sample, for example, by a suitably qualified NMR technician. Typically, this would require about fifteen minutes.

In a third step, the NMR spectrum is analysed and/or classified using a method of the present invention, as described herein. This may be performed, for example, using a computer system or device, such as a computer or linked computers, operatively configured to implement the methods described herein. In one embodiment, this step is performed at a location remote from the previous step. For example, an NMR spectrometer located in a hospital or clinic may be linked, for example, by ethernet, internet, or wireless connection, to a remote computer which performs the analysis/classification. If appropriate, the result is then forwarded to the appropriate destination, e.g., the attending physician. Typically, this would require about fifteen minutes.

Applications

The methods described herein can be used in the analysis of chemical, biochemical, and biological data.

The methods described herein provide powerful means for the diagnosis and prognosis of disease, for assisting medical practitioners in providing optimum therapy for disease, and for understanding the benefits and side-effects of xenobiotic compounds thereby aiding the drug development process.

Furthermore, the methods described herein can be applied in a non-medical setting, such as in post mortem examinations, forensic science, and the analysis of complex chemical mixtures other than mammalian cells or biofluids.

Examples of these and other applications of the methods described herein include, but are not limited to, the following:

Medical Diagnostic Applications

(a) Early detection of abnormality/problem. For example, the technique can be used to identify subjects suffering from cerebral edema immediately on arrival in the acute emergency department of a hospital. At present, when patients present with head trauma, it is difficult to tell whether cerebral edema will be a problem: as a result, it may not be possible to intervene until clinical symptoms of cerebral edema become evident, which may be too late to save the patient.

In a similar example, patients arriving at acute emergency departments can be screened for internal bleeding and organ rupture, to facilitate early surgical intervention.

In a third example, the methods described herein can be used to identify a clinically silent disease (e.g., low bone mineral density (e.g., osteoporosis); infection with Helicobacter Pylori) prior to the onset of clinical symptoms (e.g., fracture; development of ulcers).

(b) Diagnosis (identification of disease), especially cheap, rapid, and non-invasive diagnosis. For example, the methods described herein can be used to replace treadmill exercise tests, echiocardiograms, electrocardiograms, and invasive angiography as the collective method for the identification of coronary heart disease. Since the current tests for coronary heart disease are slow, expensive, and invasive (with associated morbidity and mortality), the methods described herein offer significant advantages.

(c) Differential diagnosis, e.g., classification of disease, severity of disease, etc., for example, the ability to distinguish patients with coronary artery disease affecting 1,2, or all 3 coronary arteries (see example below); the ability to distinguish disease at different anatomical sites, e.g., in the left coronary artery versus the circumflex artery, or in the carotid arteries as opposed to the coronary arteries.

(d) Population targeting. A condition (e.g., coronary heart disease, osteoporosis) may be clinically silent for many years prior to an acute event (e.g., heart attack, bone fracture), which may have significant associated morbidity or mortality. Drugs may exist to help prevent the acute event (e.g., statins for heart disease, bisphosphonates for osteoporosis), but often they cannot be efficiently targeted at the population level. The requirements for a test to be useful for population screening are that they must be cheap and non-invasive. The methods described herein are ideally suited to population screening. Screens for multiple diseases with a single blood sample (e.g., osteoporosis, heart disease, and cancer) further improve the cost/benefit ratio for screening.

(e) Classification, fingerprinting, and diagnosis of metabolic diseases (e.g., inborn errors of metabolism).

(f) Identifying, classifying, determining the progress of, and monitoring the treatment of, infectious diseases.

(g) Characterization and identification of drugs used in overdose. For example, a patient may be unconscious following an overdose and/or the nature of the drug taken in overdose may not be known. The methods described herein can be used to characterise the biological consequences of the overdose and to rapidly identify candidate agents, facilitating rapid intervention to reverse the effects. Thus an overdose of opioids could rapidly be countered with naloxone.

(h) Characterization and identification of poisons, and the metabolic or biological consequences of poisoning. Many victims of poisoning (e.g., children) are unaware of the nature of the substance they have taken. Furthermore, the subject may be unconscious or unable to communicate. The methods described herein can be used to characterise the biological consequences of the poisoning and to rapidly identify candidate poisons. This would facilitate administration of appropriate antidote, which typically must be done as quickly as possible after exposure to (e.g., ingestion of) the toxic substance.

Medical Prognosis Applications

(a) Prognosis (prediction of future outcome), including, for example, analysis of “old” samples to effect retrospective prognosis. For example, a sample can be used to assess the risk of myocardial infarction among sufferers of angina, permitting a more aggressive therapeutic strategy to be applied to those at greatest risk of progressing to a heart attack.

(b) Risk assessment, to identify people at risk of suffering from a particular indication. The methods described herein can be used for population screening (as for diagnosis) but in this case to screen for the risk of developing a particular disease. Such an approach will be useful where an effective prophylaxis is known but must be applied prior to the development of the disease in order to be effective. For example, bisphosphonates are effective at preventing bone loss in osteoporosis but they do not increase pathologically low bone mineral density. Ideally, therefore, these drugs are applied prior to any bone loss occurring. This can only be done with a technique which facilitates prediction of future disease (prognosis). The methods described herein can be used to identify those people at high risk of losing bone mineral density in the future, so that prophylaxis may begin prior to disease inception.

(c) Antenatal screening for a wide range of disease susceptibilities. The methods described herein can be used to analyse blood or tissue drawn from a pre-term fetus (e.g., during chorionic vilus sampling or amniocentesis) for the purposes of antenatal screening.

Aids to Theraputic Intervention

(a) Therapeutic monitoring, e.g., to monitor the progress of treatment. For example, by making serial diagnostic tests, it will be possible to determine whether and to what extent the subject is returning to normal following initiation of a therapeutic regimen.

(b) Patient compliance, e.g., monitoring patient compliance with therapy. Patient compliance is often very poor, particularly with therapies that have significant side-effects. Patients often claim to comply with the therapeutic regimen, but this may not always be the case. The methods described herein permit the patient compliance to be monitored, both by directly measuring the drug concentration and also by examining biological consequences of the drug. Thus, the methods described herein offer significant advantages over existing methods of monitoring compliance (such as measuring plasma concentrations of the drug) since the patient may take the drug just prior to the investigation, while having failed to comply for previous weeks or months. By monitoring the biological consequences of therapy, it is possible to assess long-term compliance.

(c) Toxicology, including sophisticated monitoring of any adverse reactions suffered, e.g., on a patient-by-patient basis. This will facilitate investigation of idiosyncratic toxicity. Some patients may suffer real, clinically significant side-effects from a therapy which were not seen in the majority. Application of the methods described herein facilitate rapid identification of these rare, idiosyncratic toxicities so that the therapy can be discontinued or modified as appropriate. Such an approach allows the therapy to be tailored to the individual metabolism of each patient.

(d) The methods described herein can be used for “pharmacometabonomics,” in analogy to pharmacogenomics, e.g., subjects could be divided into “responders” and “nonresponders” using the metabonomic profile as evidence of “response,” and features of the metabonomic profile could then be used to target future patients who would likely respond to a particular therapeutic course. For example, patients given statins could be monitored using the methods described herein for beneficial changes in the subtle composition of the lipoproteins which are associated with coronary heart disease. On this basis, the patients could be categorised into “statin responsive” or “statin unresponsive”. In a second stage, the methods described herein could be re-applied to the untreated metabonomic fingerprint to identify pattern elements which predict future responses to statins. Thus, the clinician would know whether or other patients should be treated with statins, without having to wait weeks or months to assess the outcome.

Tools for Drug Development

(a) Clinical evaluations of drug therapy and efficacy. As for therapeutic monitoring, the methods described herein can be used as one end-point in clinical trials for efficacy of new therapies. The extent to which sequential diagnostic fingerprints move towards normal can be used as one measure of the efficacy of the candidate therapy.

(b) Detection of toxic side-effects of drugs and model compounds (e.g., in the drug development process and in clinical trials). For example, it will be possible to identify the major sites of toxic effects (e.g., liver, kidney, etc.) for new treatments during Phase I studies, as well as identifying idiosyncratic toxicities during later stage clinical trials.

(c) Improvement in the quality control of transgenic animal models of disease; aiding the design of transgenic models of disease. Transgenic models of various diseases have been useful for the preclinical development of new therapies. Although the transgenic model-may recapitulate many of the phenotypic markers of the human disease, it is often unclear whether similar biochemical mechanisms underlie the resulting phenotype.

(d) Other animal models of disease. For example, injection of bovine type II collagen into mice has often been used as model of rheumatoid arthritis, resulting in joint swelling and autoantbodies, but the mechanisms resulting in the phenotype have little in common with the human disease. As a result, therapies which are effective in the animal model may be ineffective in man. The methods described herein can be used to examine the metabolic and phenotypic consequences of gene manipulation or other interventions used to yield an animal model of disease, and to compare those with the metabolic and phenotypic changes characteristic of the disease in man, and thereby validate a range of animal models of human diseases.

(e) Searching for new biochemical markers of disease and/or tissue or organ damage. For example, the NMR bin around δ3.22 was identified as being particularly associated with coronary heart disease (see examples below), and the associated species has been identified as a novel metabolic marker of coronary heart disease which may be amenable to therapeutic intervention.

Commercial and Other Non-Medical Applications

(a) Commercial classification for actuarial assessment, to address the commercial need for insurance companies to assess future risk of disease. Examples include the provision of health insurance and general life cover. This application is similar to prognostic assessment and risk assessment in population screening, except that the purpose is to provide accurate actuarial information.

(b) Clinical trial enrollment, to address the commercial need for the ability to select individuals suffering from, or at risk of suffering from, a particular condition for enrolment in clinical trials. For example, at present to perform a clinical trial to assess efficacy of a drug intended to prevent heart disease it would be necessary to enroll at least 4,000 subjects and follow them for 4 years. If it were possible to select individuals who were suffering from heart disease, it is estimated that it would be possible to use 400 subjects followed for 2 years reducing the cost by 25fold or more.

(c) Characterization and identification of illicit drugs, and the metabolic or biological consequences of substance abuse. As for monitoring patient compliance with desired therapeutics, the methods described herein can be used to examine the metabolic consequences of illegal substance abuse, permitting confirmation of the use of the substance, even if none of the substance or its metabolites are present in the system at the time of investigation. This circumvents the ability to use proscribed substances chronically, but to temporally suspend their use to avoid being identified. This application could be applied to identification of habitual users of illegal drugs (such as heroin, cocaine, amphetamines, etc.) for police use, or for monitoring use of banned substances in sports (e.g., to detect use of anabolic steroids among athletes, etc.).

(d) Application to pathology and post-mortem studies. For example, the methods described herein could be used to identify the proximate cause of death in a subject undergoing post-mortem examination.

(e) Application to forensic science. For example, the methods described herein can be used to identify the metabolic consequences of a range of actions on a subject (who may be either dead or alive at the time of the investigation). For example, the methods described herein can be applied to identify metabolic consequences of asphyxiation, poisoning, sexual arousal, or fear.

(f) Analysis of samples other than mammalian cells or biofluids. For example, the methods described herein can be applied to a panel of wines, classified by experts for their quality. By recognising patterns associated with good quality, the methods described herein can be used by wine manufacturers during the preparation of blends, as well as by wine purchasers to facilitate a rapid and independent assessment of the quality of a given wine.

(g) The methods described herein can also be used to identify (known or novel) genotypes and/or phenotypes, and to determine an organism's phenotype or genotype. This may assist with the choice of a suitable treatment or facilitate assessment of its relevance in a drug development process. For example, the generation of metabonomic data in panels of individuals with disease states, infected states, or undergoing treatment may indicate response profiles of groups of individuals which can be differentiated into two or more subgroups, indicating that an allelic genetic basis for response to the disease, state, or treatment exists. For example, a particular phenotype may not be susceptible to treatment with a certain drug, while another phenotype may be susceptible to treatment. Conversely, one phenotype might show toxicity because of a failure to metabolise and hence excrete a drug, which drug might be safe in another phenotype as it does not exhibit this effect. For example, metabonomic methods can be used to determine the acetylator status of an organism: there are two phenotypes, corresponding to “fast” and “slow” acetylation of drug metabolites. Phenotyping can be achieved on the basis of the urine alone (i.e., without dosing a xenobiotic), or on the basis of urine following dosing with a xenobiotic which has the potential for acetylation (e.g., galactosamine). Similar methods can also be used to determine other differences, such as other enzymatic polymorphisms, for example, cytochrome P450 polymorphism.

As shown below, the methods described herein can be used successfully to discriminate between twins, whether identical twins or non-identical twins.

The methods described herein may also be used in studies of the biochemical consequences of genetic modification, for example, in “knock-out animals” where one or more genes have been removed or made non-functional; in “knock-in” animals where one or more genes have been incorporated from the same or a different species; and in animals where the number of copies of a gene has been increased, as in the model which results in the over-expression of the beta amyloid protein in mice brains as a model for Alzheimer's disease). Genes can be transferred between bacterial, plant and animal species.

The combination of genomic, proteomic, and metabonomic data sets into comprehensive “bionomic” systems may permit an holistic evaluation of perturbed in vivo function.

The methods described herein may be used as an alternative or adjunct to other methods, e.g., the various genomic, pharmacogenomic, and proteomic methods.

### EXAMPLES

The following are examples are provided solely to illustrate the present invention and are not intended to limit the scope of the present invention, as described herein.

### Example 1

### Osteoarthritis

As discussed above, the inventors have developed novel methods (which employ multivariate statistical analysis and pattern recognition (PR) techniques, and optionally data filtering techniques) of analysing data (e.g., NMR spectra) from a test population which yield accurate mathematical models which may subsequently be used to classify a test sample or subject, and/or in diagnosis.

These techniques have been applied to the analysis of blood serum in the context of osteoarthritis. The metabonomic analysis can distinguish between individuals with and without osteoarthritis. Novel diagnostic biomarkers for osteoarthrits have been identified, and methods for associated diagnosis have been developed.

Obtaining NMR Spectra

Analysis was performed on serum samples collected from subjects under study. Serum taken from control subjects (n=40) and patients with osteoarthritis (n=29), prior to a formal diagnosis of bone disease.

The data were classified as “control” (▴) or “osteoarthritis” (●) on the basis of x-ray examination of the knee and wrist joints. The presence of any bony outgrowth into the cartilage in any of these joints defined the subject as having osteoarthritis. In marked contrast to osteoporosis, bone mineral density is not used in the clinical diagnosis of osteoarthritis, since patients with osteoarthritis can have any degree of average bone mineralisation (including pathologically low bone mineral density) although on average patients with osteoarthritis have slightly higher bone mineral density than control subjects.

Blood was drawn from each patient, allowed to clot in plastic tubes for 2 hours at room temperature, and the serum was collected by centrifugation. Aliquots of serum were stored at −80° C. until assayed.

Prior to NMR analysis, samples (150 μU) were diluted with solvent solution (10% D₂O v/v, 0.9% NaCl w/v) (350 μl). The diluted samples were then placed in 5 mm high quality NMR tubes (Goss Scientific Instruments Ltd).

Conventional 1-D ¹H NMR spectra of the blood serum samples were measured on a Bruker DRX-600 spectrometer using the conditions set forth in the section entitled “NMR Experimental Parameters.”

NMR Experimental Parameters

(a) General:

Samples were NON-SPINNING in the spectrometer

Temperature: 300 K

Operating Frequency: 600.22 MHz

Spectral Width: 8389.3 Hz

Number of data points (TD): 32K

Number of scans: 64

Number of dummy scans: 4 (once only, before the start of the acquisition).

Acquisition time: 1.95 s

(b) Pulse Sequence:

noesypr1d (Bruker standard noesypresat sequence, as listed in their manual): RD-90°-t₁-90°-tₘ-90°-FID

Relaxation delay (RD): 1.5 s

Fixed interval (t₁): 4 μs

Mixing time (tₘ): 150 ms

90° pulse length: 10.9 μs

Total recycle period: 3.6 s

Secondary irradiation at the water resonance during RD and tₘ

(c) Phase Cycling

The phase of the RF pulses and the receiver was cycled on successive scans to remove artefacts according to the following scheme, where PH1 refers to the first 90° pulse, PH2 refers to the second, PH3 refers to the third and PH31 refers to the phase of the receiver. In the following scheme:

0 denotes 0° phase increment

1 denotes 90° phase increment

2 denotes 180° phase increment

3 denotes 270° phase increment

PH1=02

PH2=0 0 0 0 0 0 0 0 2 2 2 2 2 2 2 2

PH3=0 0 2 2 1 1 3 3

PH31=0 2 2 0 1 3 3 1 2 0 0 2 3 1 1 3

(d) Processing of the FIDs:

This was done using using XWINNMR (version 2.1, Bruker GmbH, Germany).

Automatic zero fill×2 at end of FID.

Line broadening by multiplying the FID by a negative exponential equivalent to a line broadening of +0.3 Hz.

Fourier transform.

(e) Processing of the NMR spectra:

This was done using using XWINNMR (version 2.1, Bruker GmbH, Germany).

Spectrum peak phase adjusted manually using the zero and first order parameters PHC0, PHC1.

Baseline corrected manually using the command “basl.” This allows the subtraction of baselines of various degrees of polynomial. The simplest is to subtract a constant to remove a DC offset and this was sufficient in the present case. In other cases, it can be necessary to subtract a straight line of adjustable slope or to subtract a baseline defined by a quadratic function. The possibility exists within the software for functions up to quartic in nature.

Once properly phased and baseline corrected, the full spectra showed a flat featureless baseline on both sides of the main set of signals (i.e., outside the range δ 0 to 10), and the peaks of interest showed a clear in-phase absorption profile.

¹H NMR chemical shifts in the spectra were defined relative to that of the lactate methyl group (the middle of the doublet, taken to be at δ 1.33).

(f) Reduction of the NMR spectra to descriptors

The ¹H NMR spectra in the region δ 10-δ 0.2 were segmented into 245 regions or “buckets” of equal length (δ 0.04) using AMIX (Analysis of MIXtures software, version 2.5, Bruker, Germany). The integral of the spectrum in each segment was calculated. In order to remove the effects of variation in the suppression of the water resonance, and also the effects of variation in the urea signal caused by partial cross solvent saturation via solvent exchanging protons, the region δ 6.0 to 4.5 was set to zero integral. The following AMIX profile was used:

command=bucket_{—}1d_table

input-file=<namesfile>

output_file=<mydata.amix>

left_ppm=10

right_ppm=0.2

exclude_left_ppm=6.0

exclude1_right ppm=4.5

exclude2_left_ppm=(intentionally undefined)

exclude2_right_ppm=(intentionally undefined)

bucket_width=0.04

bucket_mode=0

bucket_scale_mode=3

bucket_multiplier=0.01

bucket_output_format=2

normalization_region_left=10

normalization_region_right=0.2

The integral data were normalized to the total spectral area using Excel (Microsoft, USA). Intensity was integrated over all included regions, and each region was then divided by the total integral and multiplied by a constant (i.e., 100, so that final integrated intensities are expressed as percentages of the total intensity).

The normalized data were then exported to the SIMCA-P (version 8.0 Umetrics, Sweden) software package and each descriptor was mean-centered. All subsequent analysis was therefore performed on normalised mean-centered data.

Data Analysis

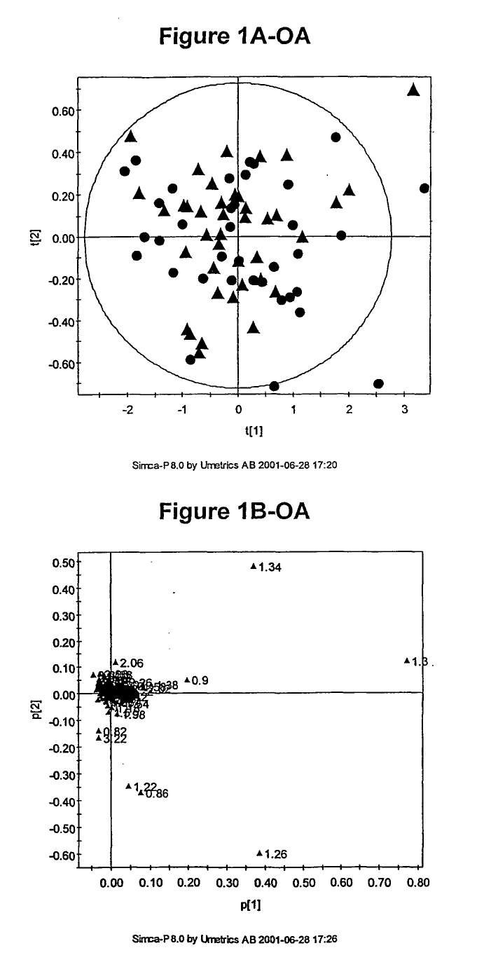

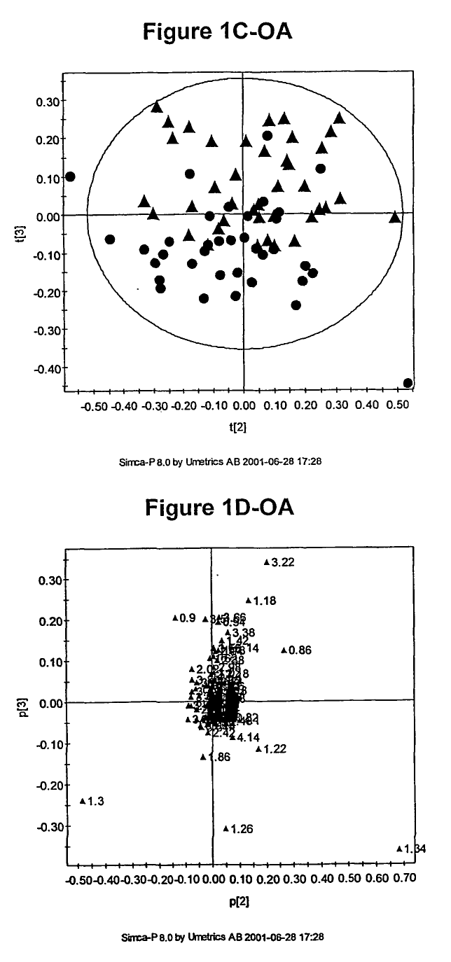

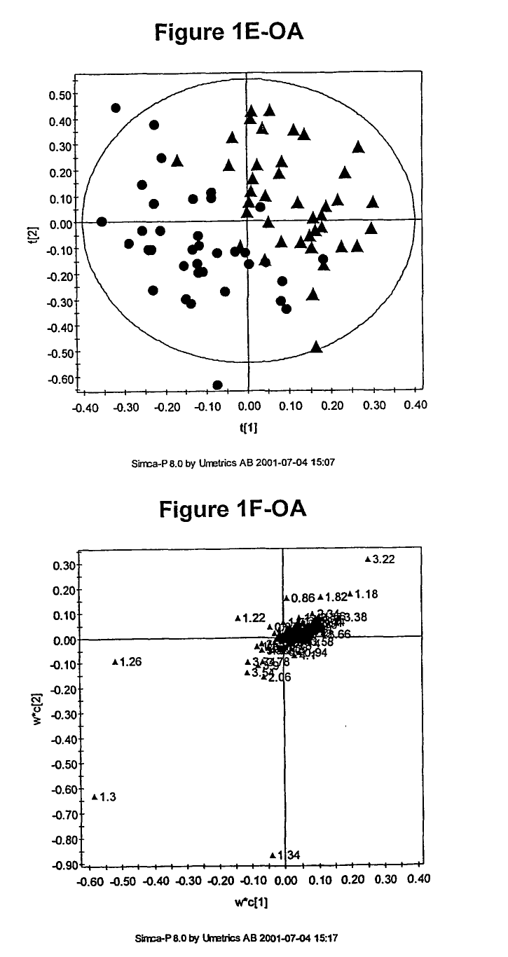

A Principal Components Analysis (PCA) model was calculated from the 1D ¹H NMR spectra of serum samples from control subjects (▴) and patients with osteoarthritis (●). The corresponding scores and loadings plots are shown in FIG. 1A-OA and FIG. 1B-OA, respectively. Those regions of the NMR spectrum which are responsible for causing separation between the different samples are also indicated in FIG. 1B-OA. Little or no separation was evident.

A Principal Components Analysis (PCA) model was calculated from the 1D ¹H NMR spectra of serum samples from control subjects (▴) and patients with osteoarthritis (●), but, in this case, prior to PCA, the data were filtered by application of orthogonal signal correction (OSC), which serves to remove variation that is not correlated to class and therefore improves subsequent data analysis. The corresponding scores and loadings plots are shown in FIG. 1C-OA and FIG. 1D-OA, respectively. PC2 vs PC3 was plotted, and the improved separation between the control and osteoarthritis samples is evident, with controls dominating the upper right of the plot and osteoarthritis dominating the lower left of the plot.

Improved separation is possible using PLS-DA (rather than the unsupervised PCA). The corresponding scores and loadings plots are shown in FIG. 1E-OA and FIG. 1F-OA, respectively. Improved separation is evident, with controls dominating the right hand side of the plot and osteoarthritis samples dominating the left hand side.

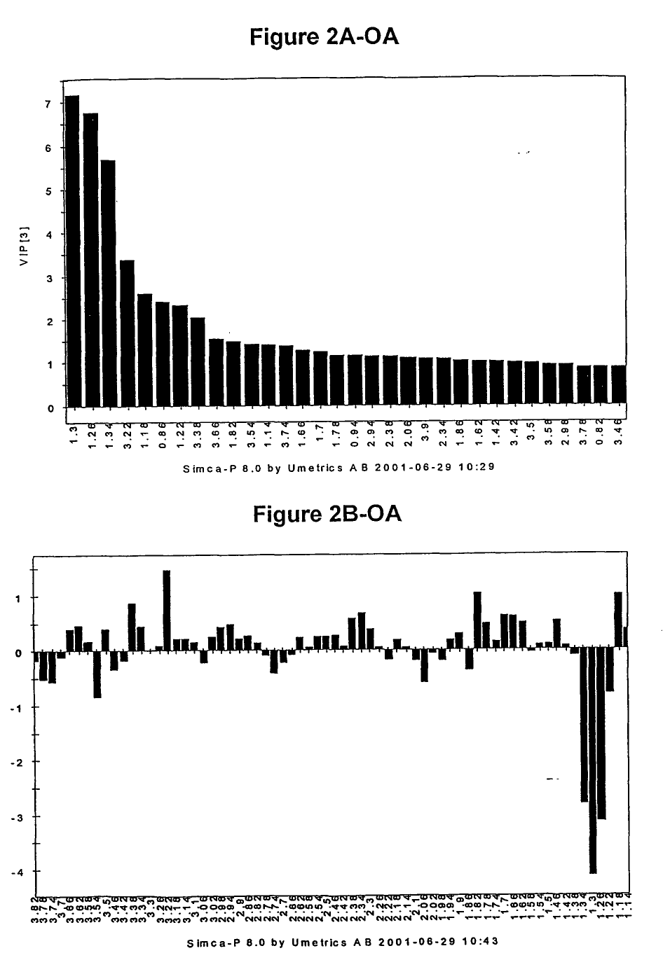

FIG. 2A-OA shows sections of the variable importance plots (VIP) and regression coefficient plots derived from the PLS-DA model described in FIG. 1E-OA.

FIG. 2B-OA shows a section of the regression coefficient plot derived from the PLS-DA model described in FIG. 1E-OA. In the regression coefficient plot, each bar represents a spectral region covering 0.04 ppm and shows how the ¹H NMR profile of one control samples differs from the ¹H NMR profile of a osteoarthritis samples. A positive value on the x-axis indicates there is a relatively greater concentration of metabolite (assigned using NMR chemical shift assignment tables) and a negative value on the x-axis indicates a relatively lower concentration of metabolite.

The 7 most important chemical shift windows for the PLS-DA model which contain NMR peaks from identified metabolites are summarised in the following table. The assignments were made by comparing the loadings with published tables of NMR data.

TABLE 1-OA

| | | | | NMR spectral |
|---|---|---|---|---|
| | Bucket | | Chem. | intensity, in |
| | Region | | Shift (ppm) | osteoarthritis |
| | (ppm) | Assignment | and Multiplicity | wrt control* |
| 1 | 1.30 | lipid | 1.30(m) | increased |
| | | CH₂ | | |
| 2 | 1.26 | lipid | 1.25(m) | increased |
| | | (CH₂)ₙ, LDL | | |
| 3 | 1.34 | predominantly lipid | 1.32(m) | increased* |
| | | CH₂CH₂CH₂CO | 1.33(d) | decreased* |
| | | also lactate CH₃ | | |
| 4 | 3.22 | choline | 3.21(s) | decreased |
| | | N(CH₃)₃ | | |
| 5 | 0.86 | lipid | 0.84(t)&0.87(t) | increased |
| | | CH₃, LDL, VLDL | | |
| 6 | 1.22 | lipid | 1.22(m) | increased |
| | | CH₃CH₂CH₂ | | |
| 7 | 3.38 | proline | 3.34(m) | decreased |
| | | half δ-CH₂ | | |

*Intensity changes of these overlapped peaks were determined by referral to the original
¹H NMR spectra.

In summary, with respect to control samples, osteoarthritis samples appear to have decreased levels of proline, choline, and 3hydroxybutyrate; and increased levels of lipids, creatine and creatinine. Additional data for the buckets associated with these species are described in the following table. Again, the assignments were made by comparing the loadings with published tables of NMR data.

TABLE 2-OA

| | | | |
|---|---|---|---|
| | | | NMR spectral |
| Bucket | | Chem. Shift | intensity, in |
| Region | | (ppm) and | osteoarthritis |
| (ppm) | Assignment | Multiplicity | wrt control* |

proline

| | | | |
|---|---|---|---|
| 3.38 | half δ-CH₂ | 3.34(m) | decreased |
| 3.46 | half δ-CH₂ | 3.45(m) | decreased |
| 3.42 | half δ-CH₂ | 3.45(m) | decreased |
| 2.34 | half β-CH₂ | 2.36(m) | decreased |
| 2.06 | half β-CH₂ | 2.05(m) | decreased |
| 2.02 | γ-CH₂ | 1.99(m) | decreased |

choline

| | | | |
|---|---|---|---|
| 3.22 | N(CH₃)₃ | 3.21(s) | decreased |
| 3.66 | NCH₂ | 3.66(m) | decreased |

3-hydroxybutyrate

| | | | |
|---|---|---|---|
| 4.14 | β-CH | 4.13(m) | decreased |
| 2.38 | half α-CH₂ | 2.38(m) | decreased |
| 2.30 | half α-CH₂ | 2.31(m) | decreased |
| 1.14 | γ-CH₃ | 1.20(d) | decreased |

lipid

| | | | |
|---|---|---|---|
| 1.34 | CH₂CH₂CH₂CO | 1.32(m) | increased |
| 1.30 | CH₂ | 1.30(m) | increased |
| 1.26 | (CH₂)ₙ, LDL | 1.25(m) | increased |
| 1.22 | CH₃CH₂CH₂ | 1.22(m) | increased |
| 0.86 | CH_{3, LDL, VLDL} | 0.84(t) & | increased |
| | | 0.87(t) | |

creatine

| | | | |
|---|---|---|---|
| 3.90 | CH₂ | 3.93(s) | increased |
| 3.02 | CH₃ | 3.04(s) | increased |

creatinine

| | | | |
|---|---|---|---|
| 4.06 | CH₂ | 4.05(s) | increased |
| 3.06 | CH₃ | 3.05(s) | increased |

The intensity changes for the proline resonance, the choline resonance at δ3.66, the lactate resonance at δ1.34 and the β-hydroxybutyrate resonance at δ4.14, all of which overlap with other peaks, were confirmed by referral to the original ¹H NMR spectra.

Validation

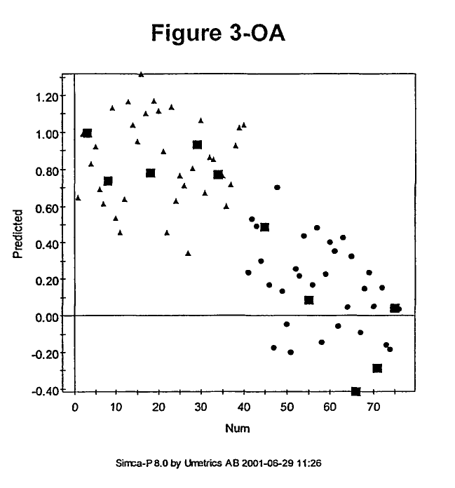

Validation was performed using a y-predicted scatter plot. FIG. 3-OA shows the y-predicted scatter plot, and hence the ability of ¹H NMR based metabonomics to predict class membership (control or osteoarthritis) of unknown samples. Using ˜85% of the control and osteoarthritis samples, a PLS-DA model was constructed and used to predict the presence of disease in the remaining 15% of samples (the validation set). The y-predicted scatter plot assigns samples to either class 1 (in this case corresponding to control) or class 0 (in this case corresponding to osteoarthritis); 0.5 is the cut-off. The PLS-DA model predicted the presence or absence of osteoporosis in 90% of cases, furthermore, for a two-component model, class can be predicted with a significance level≧70%, using a 99% confidence limit.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention as defined by the appended claims.

### References

A number of patents and publications are cited herein in order to more fully describe and disclose the invention and the state of the art to which the invention pertains. Full citations for these references are provided herein. Each of these references is incorporated herein by reference in its entirety into the present disclosure, to the same extent as if each individual reference was specifically and individually indicated to be incorporated by reference.

Ala-Korpela, M., 1995, “H-1 NMR spectroscopy of human blood plasma,” Progress in Nuclear Magnetic Resonance Spectroscopy, Vol. 27, pp. 475-554.

Ala-Korpela, M., Hiltunen, Y. and Bell, J. D., 1995, “Quantification of biomedical NMR data using artificial neural network analysis: Lipoprotein lipid profiles from H-1 NMR data of human plasma,” NMR Biomed., Vol. 8, pp. 235-244.

Andersen, C. A., 1999, “Direct orthogonalization,” Chemometrics and Intelligent Laboratory Systems, Vol. 47, pp. 51-63.

Anker, L. S., and Jurs, P. C., 1992, “Prediction of C-13 nuclear magnetic resonance chemical shifts by artificial neural networks,” Anal. Chem., Vol. 64, pp. 1157-1164.

Anthony, M. L. et al., 1994, “Pattern recognition classification of the site of nephrotoxicity based on metabolic data derived from proton nuclear magnetic resonance spectra of urine,” Mol. Pharmacol., Vol. 46, pp. 199-211.

Anthony, M. L. et al., 1995, “Classification of toxin-induced changes in ¹H NMR spectra of urine using an artificial neural network,” J. Pharm. Biomed. Anal., Vol. 13, pp. 205-211.

Beckwith-Hall, B. M. et al., 1998, “Nuclear magnetic spectroscopic and principal components analysis investigations into biochemical effects of three model hepatotoxins,” Chem. Res. Tox., Vol. 11, pp. 260-272.

Berman J. W., Guida M. P., Warren J., Amat J., and Brosnan C. F., 1996, “Localization of monocyte chemoattractant peptide-1 expression in the central nervous system in experimental autoimmune encephalomyelitis and trauma in the rat, Journal of Immunology, Vol. 156, pp 3017-3023.

Berman, J. L., Wynne, J., Cohn, P. F. (1978), ” A multivariate approach for interpreting treadmill exercise tests in coronary artery disease,” Circulation, Vol. 58, pp. 505-512.

Bishop, C., 1995, Neural Networks for Pattern Recognition, University Press, Oxford, England, pp. 164-193.

Breslow, J. L., 1993, “Transgenic mouse models of lipoprotein metabolism and atherosclerosis,” Proc. Natl. Acad. Sci. USA, Vol. 90, pp. 8314-8318.

Bretthorst, G. L., 1990a, “Bayesian Analysis. 2. Signal-Detection and Model Selection,” J. Magn. Reson., Vol. 88, pp. 552-570.

Bretthorst, G. L., 1990b, “Bayesian Analysis. 3. Applicants to NMR Signal-Detection, Model Selection, and Parameter-Estimation,” J. Magn. Reson., Vol. 88, pp. 571-595.

Bretthorst, G. L., Hung, C. C., Davignon, D. A., et al., 1988, “Bayesian-Analysis of Time-Domain Magnetic Resonance Signals,” J. Magn. Reson., Vol. 79, pp. 369-376.

Bro, R., 1997, “PARAFAC. Tutorial and applications,” in Chemometrics and Intelligent Laboratory Systems, Vol. 38, pp. 149-171.

Broomhead, D. S., and Lowe, D., 1988, “Multi-variable functional interpolation and adaptive networks,” Complex Systems, Vol. 2, pp. 321-355.

Brown, T. R. and Stoyanova, R., 1996, “NMR spectral quantitation by principal-component analysis .2. Determination of frequency and phase shifts,” J. Magn. Reson., Series B, Vol. 112, pp. 32-43.

Bruce, R. A., 1974, “The value of the Balke protocol,” Am. Heart J., Vol. 88, pp. 533-534.

Claridge, T. D. W., High-Resolution NMR Techniques in Organic Chemistry: A Practical Guide to Modern NMR for Chemists, Oxford University Press, 2000.

Collins, F. S. and McKusick, V. A., 2001, “Implications of the Human Genome Project for medical science,” JAMA, Vol. 285, pp. 540-544.

Confort-Gouny, S., Vion-Dury, J., Nicoli, F., Dano, P., Gastaut, J.-L., and Cozzone, P. J., 1992, “Metabolic characterization of neurological diseases by proton localized nmr-spectroscopy of the human brain,” Comptes Rendus de l'Academie des Sciences Serie III—Sciences de la Vie-Life Sciences, Vol. 315, pp. 287-293.

Cullen, P., Funke, H., Schulte, H. and Assmann, G., 1998, “Lipoproteins and cardiovascular risk—from genetics to CHD prevention,” European Heart Journal, Vol. 19, pp. C5C11, Suppl. C.

Despres, J., Lemieux, I., Dagenais, G., Cantin, B. and Lamarche, B., 2000, “HDL-cholesterol as a marker of coronary heart disease risk: the Québec cardiovascular study,” Atherosclerosis, Vol. 153, pp. 263-272.

Dolecek, T. A., Milas, N. C., Van Horn, L. V., Farrand, M. E., Gorder, D. D., Duchene, A. G., Dyer, J. R., Stone, P. A. and Randall, B. L, 1986, “A long-term nutrition intervention experience—lipid responses and dietary adherence patterns in the multiple risk factor intervention trial,” J. Am. Diet Assoc., Vol. 86, pp. 752-758.

Dutt, M. J. and Lee, K. H., 2000, “Proteomic analysis,” Curr. Opin. Biotechnol., Vol. 11, pp. 176-179.

Dvorak A. M., Schroeder J. T., MacGlashan D. W., Bryan K. P., Morgan E. S., Lichtenstein L. M. and MacDonald S. M., 1996, “Comparative ultrastructural morphology of human basophils stimulated to release histamine by anti-Ige, recombinant IGE-dependent histamine-releasing factor, or monocyte chemotactic protein-1”, Journal of Allergy and Clinical Immunology, Vol. 98, pp 355-370.

Eriksson, L., Johansson, E., Kettaneh-Wold, H., and Wold, S., 1999, Introduction to Multi and Megavariate Analysis using Projection Methods (PCA & PLS), UMETRICS Inc. (Box 7960, SE90719 Umea, SWEDEN), pp. 267-296.

Fan, T. W.-M., 1996, “Metabolite profiling by one- and two-dimensional NMR analysis of complex mixtures,” Prog. NMR Spectrosc., Vol. 28, pp. 161-219.

Farrant, R. D., et al., 1992, “An automatic data reduction and transfer method to aid pattern-recognition analysis and classification of NMR spectra,” J. Pharm. Biomed. Anal., Vol. 10, pp. 141-144.

Fearn, T., 2000, “On orthogonal signal correction,” Chemometrics and Intelligent Laboratory Systems, Vol. 50, pp. 47-52.

Frank, I. E., et al., 1984, “Prediction of product quality from spectral data using the partial least-squares method,” J. Chem. Info. Comp., Vol. 24, p. 20-24.

Garrod, S., Humpher, E., Connor, S. C., Connelly, J. C., Spraul, M., Nicholson, J. K., and Holmes, E., 2001, “High-resolution H-1 NMR and magic angle spinning NMR spectroscopic investigation of the biochemical effects of 2-bromoethanamine in intact renal and hepatic tissue,” Magn. Reson. Med., Vol. 45, pp. 781-790.

Gartland, K. P. R. et al., 1990a, “A pattern recognition approach to the comparison of ¹H NMR and clinical chemical data for classification of nephrotoxicity,” J. Pharm. Biomed. Anal., Vol. 8, pp. 963-968.

Gartland, K. P. R. et al., 1990b, “Pattern recognition analysis of high resolution ¹H NMR spectra of urine. A nonlinear mapping approach to the classification of toxicological data,” NMR in Biomed., Vol. 3, pp. 166-172.

Gartland, K. P. R. et al., 1991, “The application of pattern recognition methods to the analysis and classification of toxicological data derived from proton NMR spectroscopy of urine,” Mol. Pharmacol., Vol. 39, pp. 629-642.

Geisow, M. J., 1998, “Proteomics: One small step for a digital computer, one giant leap for humankind,” Nature Biotechnology, Vol. 16, p. 206.

Ghimikar R. S., Lee Y. L., He T. R., Eng L. F., 1996, “Chemokine expression in rat stab wound brain injury”, Journal of Neuroscience Research, Vol. 46, pp 727-733.

Gong J.-H.,Ratkay L. G., Waterfield J. D., and Clark-lewis I., 1997, “An antagonist of monocyte chemoattractant protein 1 (mcp-1) inhibits arthritis in the mrl-lpr mouse model”, Journal of Experimental Medicine, Vol. 186, pp 131-137.

Guyton, A. C., 1991, “Chapter 12: Electrocardiographic interpretation of cardiac muscle and coronary abnormalities,” In: A Textbook of Medical Physiology, Eighth Edition (W B Saunders, London), pp. 124-137.

Gygi, S. P.; Rochon, Y.; Franza, B. R.; Aebersold, R, 1999, “Correlation between protein and mRNA abundance in yeast,” Molecular and Cellular Biology, Vol. 19, pp. 1720-1730.

Hare, B. J., and Prestegard, J. H., 1994, “Application of neural networks to automated assignment of NMR spectra of proteins,” J. Biomol. NMR, Vol. 4, pp. 35-46.

Hiltunen, Y., Heiniemi, E. and Ala-Korpela, M., 1995, “Lipoprotein lipid quantification by neural-network analysis of H-1 NMR data from human blood-plasma,” J. Mag. Res. Ser. B, Vol. 106, pp. 191-194.

Holmes, E. et al., 1998a, “Development of a model for classification of toxin-induced lesions using ¹H NMR spectroscopy of urine combined with pattern recognition,” NMR in Biomed., Vol. 11, pp. 235-244.

Holmes, E. et al., 1998b, “The identification of novel biomarkers of renal toxicity using automatic data reduction techniques and PCA of proton NMR spectra of urine,” Chernomet. & Intel. Lab Systems, Vol. 44, pp. 245-255.

Holmes, E., et al., 1992, “NMR spectroscopy and pattern recognition analysis of the biochemical processes associated with the progression and recovery from nephrotoxic lesions in the rat induced by mercury(II)chloride and 2-bromo-ethanamine,” Mol. Pharmacol., Vol. 42, pp. 922-930.

Holmes, E., et al., 1994, “Automatic data reduction and pattern recognition methods for analysis of ¹H NMR spectra of human urine from normal and pathological states,” Anal. Biochem., Vol. 220, pp. 284-296.

Howells, S. L., Maxwell, R. J., Howe, F. A., Peet, A. C., Stubbs, M., Rodrigues, L. M., Robinson, S. P., Baluch, S., and Griffiths, J. R., 1993, “Pattern-recognition of P-31 magnetic-resonance spectroscopy tumor spectra obtained in-vivo,” NMR Biomed., Vol. 6, pp. 237-241.

Iida K, Kadota J., Kawakami K, Matsubara Y., Shirai R., and Kohno S., 1997, “Aanalysis of T cell subsets and beta chemokines in patients “with pulmonary sarcoidosis”, Thorax, Vol. 52, pp 431-437.

Isles, C. G. and Paterson, J. R., 2000, “Identifying patients at risk for coronary heart disease: implications from trials of lipid-lowering drug therapy,” Q. J. Med., Monthly Journal of the Association of Physicians, Vol. 93, pp. 567-574.

Joreskog, K. G., and Wold, H., 1982 Systems under Indirect Observation, North Holland, Amsterdam.

Kannel, W. B, Gordon, T. (eds.), February 1974, The Framingham Study, An epidemiological investigation of cardiovascular disease, DHEW pub. no. (NIH) 74-599, Public Health Service, Washington, D.C. (U.S. Government Printing Office).

Kjelsberg, M. O., Cutler, J. A. and Dolecek, T. A., 1997, “Brief description of the Multiple Risk Factor Intervention Trial,” Amer. J. Clinical Nutrition, Vol. 65 (supplement), pp. S191-S195.

Klenk, H. P., et al., 1997, “The complete genome sequence of the hyperthermophilic, sulphate-reducing archaeon Archaeoglobus fulgidus,” Nature, Vol. 390, pp. 364-370.

Kopka, P. Dormann, T. Altmann, R. N. Trethewey and L. Willmitzer, 2000, “Metabolic profiling for plant functional genomics,” Nature Biotechnology, Vol. 18, pp. 1157-1161.

Kowalski, B. R., Sharaf, M. and Illman D., Chemometrics (John Wiley & Sons, Chichester, 1986).

Kuesel, A. C., Stoyanova, R., Aiken, N. R., Ui, C.-W., Szwergold, B. S., Shaller, C. and Brown, T. R., 1996, “Quantitation of resonances in biological P-31 NMR spectra via principal component analysis: Potential and limitations,” NMR Biomed., Vol. 9, pp. 93-104.

Kuller, L. H., Ockene, J. K., Meilahn, E., Wentworth, D. N., Svendsen, K. H. and Neaton, J. D., 1991, “Cigarette-smoking and mortality,” Preventative Medicine, Vol. 20, pp. 638-654.

Kvalheim, O. M., Karstang, T. V., 1989, “Interpretation of latent-variable regression models,” Chemometrics and Intelligent Laboratory Systems, Vol. 7, pp. 39-51.

Lindon, J. C., et al., 1980, “Digitisation and Data Processing in Fourier Transform NMR,” Progress in NMR Spectroscopy, Vol. 14, pp. 27-66.

Lindon, J. C., et al., 1999, “NMR spectroscopy of biofluids,” in Annual Reports on NMR Spectroscopy (Webb, G. A., ed.), Academic Press (London), Vol. 38, pp. 1-88.

Lindon, J. C.; Holmes, E.; Nicholson, J. K., 2001, “Pattern recognition methods and applications in biomedical magnetic resonance,“Progress in NMR Spectroscopy,” Vol. 39, pp. 1-40.

Martin, G. J., 1998, “Recent advances in site-specific natural isotope fractionation studied by nuclear magnetic resonance,” Isotopes in Environmental and Health Studies, Vol. 34, pp. 233-243.

Martin, M. L. and Martin, G. J., 1999, “Site-specific isotope effects and origin inference,” Analysis, Vol. 27, p. 209-213.

Martin T. R., Galli S. J., Katona I. M. and Drazen J. M., 1989, “Role of mast-cells in anaphylaxis—evidence for the importance of mast-cells in the cardiopulmonary alterations and death induced by anti-IGE in mice”, Journal of Clinical Investigation, Vol. 83, pp 1375-1383.

Mazzucchelli L., Hauser C., Zgraggen K., Wagner H. E., Hess M. W., Laissue J. A. and Mueller C, 1996, “Differential in situ expression of the genes encoding the chemokines mcp-1 and rantes in human inflammatory bowel disease”, Journal of Pathology Vol. 178, 201-206.

McIlvain, H. E., McKinney, M. E., Thompson, A. V. and Todd, G. L., 1992, “Application of the MRFIT smoking cessation program to a healthy, mixed-sex sample,” Am. J. Prev. Med., Vol. 8, pp. 165-170.

Moka, D., et al., 1998, “Biochemical classification of kidney carcinoma biopsy samples using magic angle spinning NMR spectroscopy,” J. Pharm. Biomed. Anal., Vol. 17, pp. 125-132.

Morvan, D., Jehenson, P., Duboc, D., and Syrota, A., 1990, “Discriminant factor-analysis of P-31 NMR spectroscopic data in myopathies,” Magn. Reson. Med., Vol. 13, pp. 216-227.

Multiple Risk Factor Intervention Trial (MRFIT) Research Group, 1986, “Relationship between baseline risk factors and coronary heart disease and total mortality in the Multiple Risk Factor Intervention Trial, ” Prev. Med., Vol. 15, pp. 254-273.

Nicholson, J. K. et al., 1989, “High resolution proton magnetic resonance spectroscopy of biological fluids,” Prog. NMR Spectrosc., Vol. 21, pp. 449-501.

Nicholson, J. K. et al., 1995, “750 MHz ¹H and ¹H -_C NMR spectroscopy of human blood plasma,” Analytical Chemistry, Vol. 67, pp. 793-811.

Nicholson, J. K, et al., 1999, “Metabonomics—understanding the metabolic responses of living systems to pathophysiological stimuli via multivariate statistical analysis of biological NMR spectroscopic data,” Xenobiotica, Vol. 29, pp. 1181-1189.

Nillson, N. J., 1965, Learning Machines. McGraw-Hill, New York.

Ogata H., Takeya M., Yoshimura T., Takagi K. and Takahashi K. 1997, “The role of monocyte chemoattractant protein-1 (mcp-1) in the pathogenesis of collagen-induced arthritis in rats”, Journal of Pathology Vol. 182, pp106-114.

Parzen, E., 1962, “On estimation of a probability density function and mode,” Ann. Mathemat. Stat., Vol. 33, p.1065-1076.

Patterson, D., 1996, Artificial Neural Networks, Prentice Hall, Singapore.

Plump, A. S., Smith, J. D., Hayek, T., Aalto-Setala, K., Walsh, A., Verstuft, J. G., Rubin E. M. & Breslow, J. L., 1992, “Severe hypercholesterolemia and atherosclerosis in apolipoproteinE deficient mice created by homologous recombination in ES cells,” Cell, Vol. 71, pp. 343-353.

Press, William H., Teukolsky, Saul A., Vetterling, William T., Flannery, Brian P., January 1993, Numerical Recipes in C: The Art of Scientific Computing2nd edition, Cambridge University Press.

Quinlan, J. R., 1986, “Induction of decision trees,” Machine Learning, Vol. 1, pp. 81-106.

Ross, R., 1999, “Mechanisms of disease—Atherosclerosis—An inflammatory disease,” The New England Journal of Medicine, Vol. 340, pp. 115-126.

Sach M., Bauermeister K., Burger J., Loetscher P., Elsner J., Schollmeyer P, and Dobos G., 1997, “Inverse mcp-1/il-8 ration in effluents of CAPD patients with peritonitis and in isolated cultured human peritoneal macrophages”, Nephrology, Dialysis and Transplantation, Vol. 12, pp 315-320.

Sjostrom, M., Wold, S., and Soderstrom, B., 1986, “PLS Discriminant Plots,” Proceedings of PARC in Practice, Amsterdam, June 19-21, 1985, Elsevier Science Publishers B.V., North Holland.

Somorjai, R. L., Nikulin, A. E., Pizzi, N., Jackson, D., Scarth, G., Dolenko, B., Gordon, H., Russell, P., Lean, C. L., Delbridge, L., Mountford, C. E., and Smith, I. C. P., 1995, “Computerized consensus diagnosis—a classification strategy for the robust analysis of MR spectra .1. application to H-1 spectra of thyroid neoplasms,” Magn. Reson. Med., Vol. 33, pp. 257-263.

Speckt, D. F., 1990, “Probabilistic Neural Networks,” Neur. Networks, Vol. 3, pp. 109-118.

Spraul, M. et al., 1994, “Automatic reduction of NMR spectroscopic data for statistical and pattern recognition classification of samples,” J. Pharm. Biomed. Anal., Vol. 12, pp. 1215-1225.

Stahle, L., and Wold, S., 1987, “Partial Least Squares Analysis with Cross-Validation for the Two-Class Problem: A Monte Carlo Study,” Journal of Chemometrics, Vol. 1, pp. 185-196.

Stoyanova, R., Kuesel, A. C., and Brown, T. R., 1995, “Application of principal-component analysis for NMR spectral quantitation,” J. Magn. Reson., Series A, Vol. 115, pp. 265-269.

Sugiyama Y., Kasahara T., Mukaida N., Matsushima K. and Kitamura S., 1995, “Chemokines in bronchoalveolar lavage fluid in summer-type hypersensitivity pneumonitis”, European Respiratory Journal, Vol. 8, pp 1084-1090.

Sun, J., 1997, “Statistical analysis of NIR data: data pretreatment,” Journal of Chemometrics, Vol. 11, pp. 525-532.

Sze, D. Y., et al., 1994, “High-resolution proton NMR studies of lymphocyte extracts,” Immunomethods, Vol. 4, pp. 113-126.

Tomlins, A. M. et al., 1998, “High resolution magic angle spinning ¹H NMR analysis of intact prostatic hyperplastic and tumour tissues,” Anal. Comm., Vol. 35, pp. 113-115.

Tranter, G. E., et al., 1999, “Metabonomic prediction of drug toxicity via probabilistic neural network analysis of NMR biofluid data,” Abstr. 9^{th} North American ISSX Meeting, Oct. 24-28, 1999, p. 246.

Volejnikova S., Laskari M., Marks jr. S. C., and Graves D. T., 1997, Monocyte recruitment and expression of monocyte chemoattractant protein-1 are developmentally regulated in remodeling bone in the mouse”, American Journal of Pathology, Vol 150, pp 1711-1721.

Wasserman, P. D., 1989, Neural Computing: Theory and Practice, (Van Nostrand, ed.) Reinhold, N.Y., USA.

Weber, O. M., Duc, C. O., Meier, D., and Boesiger, P., 1998, “Heuristic optimization algorithms applied to the quantification of spectroscopic data,” Magn. Reson. Med., Vol. 39, pp. 723-730.

Westerhuis, J. A., de Jong, S., Smilde, A. K., 2001, “Direct orthogonal signal correction,” Chemometrics and Intelligent Laboratory Systems, Vol. 56, pp. 13-25.

Wise, B. M., Gallagher, N. B., 2001, http://www.eigenvector.com/MATLAB/OSC.html.

Wold, H., 1966, in Multivariate Analysis (P. R. Krishnaiah, Ed.) Academic Press, New York.

Wold, S., 1976, “Pattern recognition by means of disjoint principal components models,” Pattern Recog., Vol. 8, pp. 127-139.

Wold, S., Antti, H., Lindgren, F., and Ohman, J., 1998a, “Orthogonal Signal Correction of Near-Infrared Spectra,” Chemometrics and Intelligent Laboratory Systems, Vol. 44, pp. 175-185.

Wold, S., Kettaneh, N., Friden, H., and Holmberg, A., 1998b, “Modelling and Diagnostics of Batch Processes and Analogous Kinetic Experiments,” Chemometrics and Intelligent Laboratory Systems, Vol. 44, pp. 331-340.

Yokode, M., Hammer, R. E., Ishibashi, S., Brown, M. S. & Goldstein, J. L., 1990, “Diet-induced hypercholesterolemia in mice: prevention by over-expression of LDL receptors,” Science, Vol. 250, pp. 1273-1275.

Zeyneloglu H. B., Seli E., Senturk L. M., Gutierrez L. S., Olive D. L. and Arici A., 1998, “The effect of monocyte chemotactic protein I in intraperitoneal adhesion formation in a mouse model”, American Journal of Obstetrics and Gynecology, Vol. 179, pp 438-443.

Zheng M. H., Fan Y, Smith A, Wysocki S., Papadimitriou J. M., Wood D. J., 1998, “Gene expression of monocyte chemoattractant protein-1 in giant cell tumors of bone osteoclastoma: possible involvement in cd68⁺ macrophage-like cell migration”, Journal of Cellular Biochemistry, Vol 70, pp 121-129.

1. A method of classifying a sample, said method comprising the step of relating NMR spectral intensity at one or more predetermined diagnostic spectral windows for said sample with a predetermined condition associated with osteoarthritis.

2. A method, according to claim 1, of classifying a sample from a subject, said method comprising the step of relating NMR spectral intensity at one or more predetermined diagnostic spectral windows for said sample with a predetermined condition associated with osteoarthritis of said subject.

3. A method, according to claim 1, of classifying a sample, said method comprising the step of relating NMR spectral intensity at one or more predetermined diagnostic spectral windows for said sample with the presence or absence of a predetermined condition associated with osteoarthritis.

4. A method, according to claim 1, of classifying a sample from a subject, said method comprising the step of relating NMR spectral intensity at one or more predetermined diagnostic spectral windows for said sample with the presence or absence of a predetermined condition associated with osteoarthritis of said subject.

5. A method, according to claim 1, of classifying a sample, said method comprising the step of relating a modulation of NMR spectral intensity, relative to a control value, at one or more predetermined diagnostic spectral windows for said sample with a predetermined condition associated with osteoarthritis.

6. A method, according to claim 1, of classifying a sample from a subject, said method comprising the step of relating a modulation of NMR spectral intensity, relative to a control value, at one or more predetermined diagnostic spectral windows for said sample with a predetermined condition associated with osteoarthrits of said subject.

7. A method, according to claim 1, of classifying a sample, said method comprising the step of relating a modulation of NMR spectral intensity, relative to a control value, at one or more predetermined diagnostic spectral windows for said sample with the presence or absence of a predetermined condition associated with osteoarthritis.

8. A method, according to claim 1, of classifying a sample from a subject, said method comprising the step of relating a modulation of NMR spectral intensity, relative to a control value, at one or more predetermined diagnostic spectral windows for said sample with the presence or absence of a predetermined condition associated with osteoarthritis of said subject.

9. A method of classifying a subject, said method comprising the step of relating NMR spectral intensity at one or more predetermined diagnostic spectral windows for a sample from said subject with a predetermined condition associated with osteoarthritis of said subject.

10. A method, according to claim 9, of classifying a subject, said method comprising the step of relating NMR spectral intensity at one or more predetermined diagnostic spectral windows for a sample from said subject with the presence or absence of a predetermined condition associated with osteoarthritis of said subject.

11. A method, according to claim 9, of classifying a subject, said method comprising the step of relating a modulation of NMR spectral intensity, relative to a control value, at one or more predetermined diagnostic spectral windows for a sample from said subject with a predetermined condition associated with osteoarthrits of said subject.

12. A method, according to claim 9, of classifying a subject, said method comprising the step of relating a modulation of NMR spectral intensity, relative to a control value, at one or more predetermined diagnostic spectral windows for a sample from said subject with the presence or absence of a predetermined condition associated with osteoarthritis of said subject.

13. A method of diagnosing a predetermined condition associated with osteoarthritis of a subject, said method comprising the step of relating NMR spectral intensity at one or more predetermined diagnostic spectral windows for a sample from said subject with said predetermined condition of said subject.

14. A method, according to claim 13, of diagnosing a predetermined condition associated with osteoarthritis of a subject, said method comprising the step of relating NMR spectral intensity at one or more predetermined diagnostic spectral windows for a sample from said subject with the presence or absence of said predetermined condition of said subject.

15. A method, according to claim 13, of diagnosing a predetermined condition associated with osteoarthritis of a subject, said method comprising the step of relating a modulation of NMR spectral intensity, relative to a control value, at one or more predetermined diagnostic spectral windows for a sample from said subject with said predetermined condition of said subject.

16. A method, according to claim 13, of diagnosing a predetermined condition associated with osteoarthritis of a subject, said method comprising the step of relating a modulation of NMR spectral intensity, relative to a control value, at one or more predetermined diagnostic spectral windows for a sample from said subject with the presence or absence of said predetermined condition of said subject.

17. A method of classifying a sample, said method comprising the step of relating the amount of, or relative amount of one or more diagnostic species present in said sample with a predetermined condition associated with osteoarthritis.

18. A method, according to claim 17, of classifying a sample from a subject, said method comprising the step of relating the amount of, or relative amount of one or more diagnostic species present in said sample with a predetermined condition associated with osteoarthritis of said subject.

19. A method, according to claim 17, of classifying a sample, said method comprising the step of relating the amount of, or relative amount of one or more diagnostic species present in said sample with the presence or absence of a predetermined condition associated with osteoarthritis.

20. A method, according to claim 17, of classifying a sample from a subject, said method comprising the step of relating the amount of, or the relative amount of, one or more diagnostic species present in said sample with the presence or absence of a predetermined condition associated with osteoarthritis of said subject.

21. A method, according to claim 17, of classifying a sample, said method comprising the step of relating a modulation of the amount of, or relative amount of one or more diagnostic species present in said sample, as compared to a control sample, with a predetermined condition associated with osteoarthritis.

22. A method, according to claim 17, of classifying a sample from a subject, said method comprising the step of relating a modulation of the amount of, or relative amount of one or more diagnostic species present in said sample, as compared to a control sample, with a predetermined condition associated with osteoarthritis of said subject.

23. A method, according to claim 17, of classifying a sample, said method comprising the step of relating a modulation of the amount of, or relative amount of one or more diagnostic species present in said sample, as compared to a control sample, with the presence or absence of a predetermined condition associated with osteoarthritis.

24. A method, according to claim 17, of classifying a sample from a subject, said method comprising the step of relating a modulation of the amount of, or relative amount of one or more diagnostic species present in said sample, as compared to a control sample, with the presence or absence of a predetermined condition associated with osteoarthritis of said subject.

25. A method of classifying a subject, said method comprising the step of relating the amount of, or relative amount of one or more diagnostic species present in a sample from said subject with a predetermined condition associated with osteoarthritis of said subject.

26. A method, according to claim 25, of classifying a subject, said method comprising the step of relating the amount of, or relative amount of one or more diagnostic species present in a sample from said subject with the presence or absence of a predetermined condition associated with osteoarthritis of said subject.

27. A method, according to claim 25, of classifying a subject, said method comprising the step of relating a modulation of the amount of, or relative amount of one or more diagnostic species present in a sample from said subject, as compared to a control sample, with a predetermined condition associated with osteoarthritis of said subject.

28. A method, according to claim 25, of classifying a subject, said method comprising the step of relating a modulation of the amount of, or relative amount of one or more diagnostic species present in a sample from said subject, as compared to a control sample, with the presence or absence of a predetermined condition associated with osteoarthritis of said subject.

29. A method of diagnosing a predetermined condition associated with osteoarthritis of a subject, said method comprising the step of relating the amount of, or relative amount of one or more diagnostic species present in a sample from said subject with said predetermined condition of said subject.

30. A method, according to claim 29, of diagnosing a predetermined condition associated with osteoarthritis of a subject, said method comprising the step of relating the amount of, or relative amount of one or more diagnostic species present in a sample from said subject with the presence or absence of said predetermined condition of said subject.

31. A method, according to claim 29, of diagnosing a predetermined condition associated with osteoarthritis of a subject, said method comprising the step of relating a modulation of the amount of, or relative amount of one-or more diagnostic species present in a sample from said subject, as compared to a control sample, with said predetermined condition of said subject.

32. A method, according to claim 29, of diagnosing a predetermined condition associated with osteoarthritis of a subject, said method comprising the step of relating a modulation of the amount of, or relative amount of one or more diagnostic species present in a sample from said subject, as compared to a control sample, with the presence or absence of said predetermined condition of said subject.

33. A method of classification, said method comprising the steps of:
(a) forming a predictive mathematical model by applying a modelling method to modelling data;
(b) using said model to classify a test sample.

34. A method, according to claim 33, of classifying a test sample, said method comprising the steps of:
(a) forming a predictive mathematical model by applying a modelling method to modelling data;
wherein said modelling data comprises a plurality of data sets for modelling samples of known class;
(b) using said model to classify said test sample as being a member of one of said known classes.

35. A method, according to claim 33, of classifying a test sample, said method comprising the steps of:
(a) forming a predictive mathematical model by applying a modelling method to modelling data;
wherein said modelling data comprises at least one data set for each of a plurality of modelling samples;
wherein said modelling samples define a class group consisting of a plurality of classes;
wherein each of said modelling samples is of a known class selected from said class group; and,
(b) using said model with a data set for said test sample to classify said test sample as being a member of one class selected from said class group.

36. A method of classification, said method comprising the step of:
using a predictive mathematical model;
wherein said model is formed by applying a modelling method to modelling data;
to classify a test sample.

37. A method, according to claim 36, of classifying a test sample, said method comprising the step of:
using a predictive mathematical model;
wherein said model is formed by applying a modelling method to modelling data;
wherein said modelling data comprises a plurality of data sets for modelling samples of known class;
to classify said test sample as being a member of one of said known classes.

38. A method, according to claim 36, of classifying a test sample, said method comprising the step of:
using a predictive mathematical model;
wherein said model is formed by applying a modelling method to modelling data;
wherein said modelling data comprises at least one data set for each of a plurality of modelling samples;
wherein said modelling samples define a class group consisting of a plurality of classes;
wherein each of said modelling samples is of a known class selected from said class group;
with a data set for said test sample to classify said test sample as being a member of one class selected from said class group.

39. A method of classification, said method comprising the steps of:
(a) forming a predictive mathematical model by applying a modelling method to modelling data;
(b) using said model to classify a subject.

40. A method, according to claim 39, of classifying a subject, said method comprising the steps of:
(a) forming a predictive mathematical model by applying a modelling method to modelling data;
wherein said modelling data comprises a plurality of data sets for modelling samples of known class;
(b) using said model to classify a test sample from said subject as being a member of one of said known classes, and thereby classify said subject.

41. A method, according to claim 39, of classifying a subject, said method comprising the steps of:
(a) forming a predictive mathematical model by applying a modelling method to modelling data;
wherein said modelling data comprises at least one data set for each of a plurality of modelling samples;
wherein said modelling samples define a class group consisting of a plurality of classes;
wherein each of said modelling samples is of a known class selected from said class group; and,
(b) using said model with a data set for a test sample from said subject to classify said test sample as being a member of one class selected from said class group, and thereby classify said subject.

42. A method of classification, said method comprising the step of:
using a predictive mathematical model;
wherein said model is formed by applying a modelling method to modelling data;
to classify a subject.

43. A method, according to claim 42, of classifying a subject, said method comprising the step of:
using a predictive mathematical model
wherein said model is formed by applying a modelling method to modelling data;
wherein said modelling data comprises a plurality of data sets for modelling samples of known class;
to classify a test sample from said subject as being a member of one of said known classes, and thereby classify said subject.

44. A method, according to claim 42, of classifying a subject, said method comprising the step of:
using a predictive mathematical model,
wherein said model is formed by applying a modelling method to modelling data;
wherein said modelling data comprises at least one data set for each of a plurality of modelling samples;
wherein said modelling samples define a class group consisting of a plurality of classes;
wherein each of said modelling samples is of a known class selected from said class group;
with a data set for a test sample from said subject to classify said test sample as being a member of one class selected from said class group, and thereby classify said subject.

45. A method of diagnosis, said method comprising the steps of:
(a) forming a predictive mathematical model by applying a modelling method to modelling data;
(b) using said model to diagnose a subject.

46. A method, according to claim 45, of diagnosing a predetermined condition associated with osteoarthritis of a subject, said method comprising the steps of:
(a) forming a predictive mathematical model by applying a modelling method to modelling data;
wherein said modelling data comprises a plurality of data sets for modelling samples of known class;
(b) using said model to classify a test sample from said subject as being a member of one of said known classes, and thereby diagnose said subject.

47. A method, according to claim 45, of diagnosing a predetermined condition associated with osteoarthrits of a subject, said method comprising the steps of:
(a) forming a predictive mathematical model by applying a modelling method to modelling data;
wherein said modelling data comprises at least one data set for each of a plurality of modelling samples;
wherein said modelling samples define a class group consisting of a plurality of classes;
wherein each of said modelling samples is of a known class selected from said class group; and,
(b) using said model with a data set for a test sample from said subject to classify said test sample as being a member of one class selected from said class group, and thereby diagnose said subject.

48. A method of diagnosis, said method comprising the step of:
using a predictive mathematical model;
wherein said model is formed by applying a modelling method to modelling data;
to diagnose a subject.

49. A method, according to claim 48, of diagnosing a predetermined condition associated with osteoarthritis of a subject, said method comprising the step of:
using a predictive mathematical model;
wherein said model is formed by applying a modelling method to modelling data;
wherein said modelling data comprises a plurality of data sets for modelling samples of known class;
to classify a test sample from said subject as being a member of one of said known classes, and thereby diagnose said subject.

50. A method, according to claim 48, of diagnosing a predetermined condition associated with osteoarthritis of a subject, said method comprising the step of:
using a predictive mathematical model;
wherein said model is formed by applying a modelling method to modelling data;
wherein said modelling data comprises at least one data set for each of a plurality of modelling samples;
wherein said modelling samples define a class group consisting of a plurality of classes;
wherein each of said modelling samples is of a known class selected from said class group;
with a data set for a test sample from said subject to classify said test sample as being a member of one class selected from said class group, and thereby diagnose said subject.

51. A method according to any one of claims 1 to 50, wherein said test sample is a test sample from a subject, and said predetermined condition is a predetermined condition of said subject.

52. A method according to any one of claims 1 to 50, wherein said “a modulation of” is “an increase or decrease in.”

53. A method according to any one of claims 1 to 52, wherein said relating step involves the use of a predictive mathematical model.

54. A method according to any one of claims 1 to 52, wherein said modelling method is a multivariate statistical analysis modelling method.

55. A method according to any one of claims 1 to 52, wherein said modelling method is a multivariate statistical analysis modelling method which employs a pattern recognition method.

56. A method according to any one of claims 1 to 52, wherein said modelling method is, or employs PCA.

57. A method according to any one of claims 1 to 52, wherein said modelling method is, or employs PLS.

58. A method according to any one of claims 1 to 52, wherein said modelling method is, or employs PLS-DA.

59. A method according to any one of claims 1 to 58, wherein said modelling method includes a step of data filtering.

60. A method according to any one of claims 1 to 58, wherein said modelling method includes a step of orthogonal data filtering.

61. A method according to any one of claims 1 to 58, wherein said modelling method includes a step of OSC.

62. A method according to any one of claims 1 to 61, wherein said model takes account of one or more diagnostic species.

63. A method according to any one of claims 1 to 62, wherein said modelling data comprise spectral data.

64. A method according to any one of claims 1 to 62, wherein said modelling data comprise both spectral data and non-spectral data.

65. A method according to any one of claims 1 to 62, wherein said modelling data comprise NMR spectral data.

66. A method according to any one of claims 1 to 62, wherein said modelling data comprise both NMR spectral data and non-NMR spectral data.

67. A method according to any one of claims 1 to 62, wherein said NMR spectral data comprises ¹H NMR spectral data and/or ¹³C NMR spectral data.

68. A method according to any one of claims 1 to 62, wherein said NMR spectral data comprises ¹H NMR spectral data.

69. A method according to any one of claims 1 to 62, wherein said modelling data comprise spectra.

70. A method according to any one of claims 1 to 62, wherein said modelling data are spectra.

71. A method according to any one of claims 1 to 70, wherein said modelling data comprises a plurality of data sets for modelling samples of known class.

72. A method according to any one of claims 1 to 70, wherein said modelling data comprises at least one data set for each of a plurality of modelling samples.

73. A method according to any one of claims 1 to 70, wherein said modelling data comprises exactly one data set for each of a plurality of modelling samples.

74. A method according to any one of claims 1 to 70, wherein said using step is:
using said model with a data set for said test sample to classify said test sample as being a member of one class selected from said class group.

75. A method according to any one of claims 1 to 74, wherein each of said data sets comprises spectral data.

76. A method according to any one of claims 1 to 74, wherein each of said data sets comprises both spectral data and non-spectral data.

77. A method according to any one of claims 1 to 74, wherein each of said data sets comprises NMR spectral data.

78. A method according to any one of claims 1 to 74, wherein each of said data sets comprises both NMR spectral data and non-NMR spectral data.

79. A method according to any one of claims 1 to 74, wherein said NMR spectral data comprises ¹H NMR spectral data and/or ¹³C NMR spectral data.

80. A method according to any one of claims 1 to 74, wherein said NMR spectral data comprises ¹H NMR spectral data.

81. A method according to any one of claims 1 to 74, wherein each of said data sets comprises a spectrum.

82. A method according to any one of claims 1 to 74, wherein each of said data sets comprises a ¹H NMR spectrum and/or ¹³C NMR spectrum.

83. A method according to any one of claims 1 to 74, wherein each of said data sets comprises a ¹H NMR spectrum.

84. A method according to any one of claims 1 to 74, wherein each of said data sets is a spectrum.

85. A method according to any one of claims 1 to 74, wherein each of said data sets is a ¹H NMR spectrum and/or ¹³C NMR spectrum.

86. A method according to any one of claims 1 to 74, wherein each of said data sets is a ¹H NMR spectrum.

87. A method according to any one of claims 1 to 86, wherein said non-spectral data is non-spectral clinical data.

88. A method according to any one of claims 1 to 86, wherein said non-NMR spectral data is non-spectral clinical data.

89. A method according to any one of claims 1 to 88, wherein said class group comprises classes associated with said predetermined condition.

90. A method according to any one of claims 1 to 88, wherein said class group comprises exactly two classes.

91. A method according to any one of claims 1 to 88, wherein said class group comprises exactly two classes: presence of said predetermined condition; and absence of said predetermined condition.

92. A method according to any one of claims 1 to 91, wherein said sample is an in vivo sample.

93. A method according to any one of claims 1 to 91, wherein said sample is an ex vivo sample.

94. A method according to any one of claims 1 to 91, wherein said sample is a blood sample or a blood-derived sample.

95. A method according to any one of claims 1 to 91, wherein said sample is a blood sample.

96. A method according to any one of claims 1 to 91, wherein said sample is a blood plasma sample.

97. A method according to any one of claims 1 to 91, wherein said sample is a blood serum sample.

98. A method according to any one of claims 1 to 97, wherein said subject is an animal.

99. A method according to any one of claims 1 to 97, wherein said subject is a mammal.

100. A method according to any one of claims 1 to 97, wherein said subject is a human.

101. A method according to any one of claims 1 to 100, wherein said one or more predetermined diagnostic spectral windows is: a single predetermined diagnostic spectral window.

102. A method according to any one of claims 1 to 100, wherein said one or more predetermined diagnostic spectral windows is: a plurality of predetermined diagnostic spectral windows.

103. A method according to any one of claims 1 to 100, wherein
said one or more predetermined diagnostic spectral windows is: a plurality of diagnostic spectral windows, and,
said NMR spectral intensity at one or more predetermined diagnostic spectral windows is: a combination of a plurality of NMR spectral intensities, each of which is NMR spectral intensity for one of said plurality of predetermined diagnostic spectral windows.

104. A method according to claim 103, wherein said combination is a linear combination.

105. A method according to any one of claims 1 to 104, wherein said one-or more predetermined diagnostic spectral windows are associated with one or more diagnostic species.

106. A method according to any one of claims 1 to 104, wherein at least one of said one or more predetermined diagnostic spectral windows encompasses a chemical shift value for an NMR resonance of a diagnostic species.

107. A method according to any one of claims 1 to 104, each of a plurality of said one or more predetermined diagnostic spectral windows encompasses a chemical shift value for an NMR resonance of a diagnostic species.

108. A method according to any one of claims 1 to 104, each of said one or more predetermined diagnostic spectral windows encompasses a chemical shift value for an NMR resonance of a diagnostic species.

109. A method according to any one of claims 106 to 108, wherein said NMR resonance is a ¹H NMR resonance.

110. A method according to any one of claims 1 to 109, wherein said one or more diagnostic species are endogenous diagnostic species.

111. A method according to any one of claims 1 to 1109, wherein said one or more diagnostic species are associated with NMR spectral intensity at predetermined diagnostic spectral windows.

112. A method according to any one of claims 1 to 111, said one or more diagnostic species are a plurality of diagnostic species.

113. A method according to any one of claims 1 to 111, said one or more diagnostic species is a single diagnostic species.

114. A method according to any one of claims 1 to 113, wherein said classification is performed on the basis of an amount, or a relative amount, of a single diagnostic species.

115. A method according to any one of claims 1 to 113, wherein said classification is performed on the basis of an amount, or a relative amount, of a plurality of diagnostic species.

116. A method according to any one of claims 1 to 113, wherein said classification is performed on the basis of an amount, or a relative amount, of each of a plurality of diagnostic species.

117. A method according to any one of claims 1 to 113, wherein said classification is performed on the basis of a total amount, or a relative total amount, of a plurality of diagnostic species.

118. A method according to any one of claims 1 to 113, wherein:
said one or more diagnostic species is: a plurality of diagnostic species; and,
said amount of, or relative amount of one or more diagnostic species is: a combination of a plurality of amounts, or relative amounts, each of which is the amount of, or relative amount of one of said plurality of diagnostic species.

119. A method according to claim 118, wherein said combination is a linear combination.

120. A method according to any one of claims 1 to 119, wherein said predetermined diagnostic spectral windows are defined by one or more index values, δᵣ, corresponding to the bucket regions listed in Table 1-OA and/or Table 2-OA.

121. A method according to any one of claims 1 to 119, wherein at least one of said one or more predetermined diagnostic species is a species described in Table 1-OA and/or Table 2-OA.

122. A method according to any one of claims 1 to 119, wherein each of a plurality of said one or more predetermined diagnostic species is a species described in Table 1-OA and/or Table 2-OA.

123. A method according to any one of claims 1 to 119, wherein each of said one or more predetermined diagnostic species is a species described in Table 1-OA and/or Table 2-OA.

124. A method of identifying a diagnostic species, or a combination of a plurality of diagnostic species, for a predetermined condition associated with osteoarthritis, said method comprising the steps of:
(a) applying a multivariate statistical analysis method to experimental data;
wherein said experimental data comprises at least one data comprising experimental parameters measured for each of a plurality of experimental samples;
wherein said experimental samples define a class group consisting of a plurality of classes;
wherein at least one of said plurality of classes is a class associated with said predetermined condition, e.g., a class associated with the presence of said predetermined condition;
wherein at least one of said plurality of classes is a class not associated with said predetermined condition, e.g., a class associated with the absence of said predetermined condition;
wherein each of said experimental samples is of known class selected from said class group;
and:
(b) identifying one or more critical experimental parameters;
wherein each of said critical experimental parameters is statistically significantly different for classes of said class group, e.g., is statistically significant for discriminating between classes of said class group; and,
(c) matching each of one or more of said one or more critical experimental parameters with said diagnostic species;
or:
(b) identifying a combination of a plurality of critical experimental parameters;
wherein said combination of a plurality of critical experimental parameters is statistically significantly different for classes of said class group, e.g., is statistically significant for discriminating between classes of said class group;
and,
(c) matching each of one or more of said plurality of critical experimental parameters with said combination of a plurality of diagnostic species.

125. A method, according to claim 124, wherein:
one or more of said critical experimental parameters is a spectral parameter; and
said identifying and matching steps are:
(b) identifying one or more critical experimental spectral parameters; and,
(c) matching each of one or more of said one or more critical experimental spectral parameters with a spectral feature, e.g., a spectral peak;
and matching one or more of said spectral peaks with said diagnostic species;
or:
(b) identifying a combination of a plurality of critical experimental spectral parameters; and,
(c) matching each of a plurality of said plurality of critical experimental spectral parameters with a spectral feature, e.g., a spectral peak;
and matching one or more of said spectral peaks with said combination of a plurality of diagnostic species.

126. A method according to any one of claims 124 to 125, wherein said multivariate statistical analysis method is a multivariate statistical analysis method which employs pattern recognition method.

127. A method according to any one of claims 124 to 126, wherein said multivariate statistical analysis method is, or employs PCA.

128. A method according to any one of claims 124 to 126, wherein said multivariate statistical analysis method is, or employs PLS.

129. A method according to any one of claims 124 to 126, wherein said multivariate statistical analysis method is, or employs PLS-DA.

130. A method according to any one of claims 124 to 129, wherein said multivariate statistical analysis method includes a step of data filtering.

131. A method according to any one of claims 124 to 129, wherein said multivariate statistical analysis method includes a step of orthogonal data filtering.

132. A method according to any one of claims 124 to 129, wherein said multivariate statistical analysis method includes a step of OSC.

133. A method according to any one of claims 124 to 132, wherein said experimental parameters comprise spectral data.

134. A method according to any one of claims 124 to 132, wherein said experimental parameters comprise both spectral data and non-spectral data.

135. A method according to any one of claims 124 to 132, wherein said experimental parameters comprise NMR spectral data.

136. A method according to any one of claims 124 to 132, wherein said experimental parameters comprise both NMR spectral data and non-NMR spectral data.

137. A method according to any one of claims 124 to 136, wherein said NMR spectral data comprises ¹H NMR spectral data and/or ¹³C NMR spectral data.

138. A method according to any one of claims 124 to 136, wherein said NMR spectral data comprises ¹H NMR spectral data.

139. A method according to any one of claims 124 to 138, wherein said non-spectral data is non-spectral clinical data.

140. A method according to any one of claims 124 to 138, wherein said non-NMR spectral data is non-spectral clinical data.

141. A method according to any one of claims 124 to 140, wherein said critical experimental parameters are spectral parameters.

142. A method according to any one of claims 124 to 141, wherein said class group comprises classes associated with said predetermined condition.

143. A method according to any one of claims 124 to 142, wherein said class group comprises exactly two classes.

144. A method according to any one of claims 124 to 142, wherein said class group comprises exactly two classes: presence of said predetermined condition; and absence of said predetermined condition.

145. A method according to any one of claims 124 to 142, wherein said class associated with said predetermined condition is a class associated with the presence of said predetermined condition.

146. A method according to any one of claims 124 to 142, wherein said class not associated with said predetermined condition is a class associated with the absence of said predetermined condition.

147. A method according to any one of claims 124 to 146, said method further comprising the additional step of:
(d) confirming the identity of said diagnostic species.

148. A computer system or device, such as a computer or linked computers, operatively configured to implement a method according to any one of claims 1 to 147.

149. Computer code suitable for implementing a method according to any one of claims 1 to 147 on a suitable computer system.

150. A computer program comprising computer program means adapted to perform a method according to according to any one of claims 1 to 147, when said program is run on a computer.

151. A computer program according to claim 150, embodied on a computer readable medium.

152. A data carrier which carries computer code suitable for implementing a method according to any one of claims 1 to 147 on a suitable computer.

153. Computer code and/or computer readable data representing a predictive mathematical model as described in any one of claims 1 to 147.

154. A data carrier which carries computer code and/or computer readable data representing a predictive mathematical model as described in any one of claims 1 to 147.

155. A computer system or device, such as a computer or linked computers, programmed or loaded with computer code and/or computer readable data representing a predictive mathematical model as described in any one of claims 1 to 147.

156. A system comprising:
(a) a first component comprising a device for obtaining NMR spectral intensity data for a sample; and,
(b) a second component comprising computer system or device, such as a computer or linked computers, operatively configured to implement a method according to any one of claims 1 to 147, and operatively linked to said first component.

157. A diagnostic species identified by a method according to any one of claims 124 to 147.

158. A diagnostic species identified by a method according to any one of claims 124 to 147 for use in a method of classification.

159. A method of classification which employs or relies upon one or more diagnostic species identified by a method according to any one of claims 124 to 147.

160. Use of one or more diagnostic species identified by a method of classification according to any one of claims 124 to 147.

161. An assay for use in a method of classification, which assay relies upon one or more diagnostic species identified by a method according to any.-one of claims 124 to 147.

162. Use of an assay in a method of classification, which assay relies upon one or more diagnostic species identified by a method according to any one of claims 124 to 147.

163. A method of therapeutic monitoring of a subject undergoing therapy which employs a method of classification according to any one of claims 1 to 123.

164. A method of evaluating drug therapy and/or drug efficacy which employs a method of classification according to any one of claims 1 to 123.
